(19) United States
(12) Patent Application Publication
(10) Pub. No.: US 2026/0265383 A1
(43) Pub. Date: Sep. 10, 2026

(54) ANTIBODIES AND USES THEREOF

(71) Applicant: FUSION ANTIBODIES PLC, Belfast Antrim (GB)

(72) Inventors: Richard John BUICK, Belfast Antrim (GB); Anthony Arthur O'KANE, Belfast (GB); Maria Eugenia RIVEIRO, Boulogne-Billancourt (FR); Christopher MURRAY, Belfast Antrim (GB)

(73) Assignee: FUSION ANTIBODIES PLC, Belfast Antrim (GB)

(21) Appl. No.: 19/163,833

(22) PCT Filed: Mar. 11, 2024
(86) PCT No.: PCT/GB2024/050657

§ 371 Date: Sep. 9, 2025

(30) Foreign Application Priority Data
Mar. 10, 2023 (GB) 2303531.4

(51) Int. Cl. C07K 16/28; A61P 35/00

(52) U.S. Cl. CPC C07K 16/2863; A61P 35/00; C07K 2317/24

(57) ABSTRACT

The invention relates to anti-RAMP2 and anti-RAMP3 antibodies which inhibit the pro-tumourigenic effects of adrenomedullin. The antibodies may be used in the treatment of cancer.

### FIELD OF THE INVENTION

This application relates to antibody molecules and their use in methods of treatment, for example methods of treatment of cancer. In particular, it relates to antibody molecules which inhibit the pro-tumourigenic effects of adrenomedullin.

### BACKGROUND TO THE INVENTION

Adrenomedullin (AM) is a regulatory peptide whose involvement in tumor progression and metastasis development has become more relevant during the last years. The whole literature supports the idea of AM as a survival factor for tumour cells, that can be produced either by the tumour itself or by several surrounding stromal cells. In general, AM expression is upregulated by hypoxia, a common occurrence in (solid) tumours, and the excessive production of this peptide is associated with poorer prognosis for the patients. AM acts as an autocrine/paracrine growth factor preventing apoptosis, increasing tumor cell motility and metastasis, inducing angiogenesis as well as blocking immuno-surveillance by inhibition of the immune system. As referred above, AM expression gets rapidly activated by hypoxia through a HIF-1α-mediated mechanism and acts as a potent angiogenic factor promoting neovascularization.

AM mediates its effects through association with the calcitonin like receptor (CLR), a 7-transmembrane domain receptor belonging to the family of G-protein-coupled receptor (GPCR) coupled to a receptor activity-modifying protein (RAMP), a single-pass transmembrane protein. RAMP family is composed of three proteins (RAMP1, RAMP2 and RAMP3) that could interact with GPCRs inducing profound changes in ligand binding and receptor pharmacology. RAMPs are essential for the binding of CLR to the cell surface. Depending on the type of RAMP associated with CLR, the resulting complexes act as receptors for CGRP or AM. By interacting with RAMP1, CLR acquires a high affinity for CGRP, whereas the interaction with either RAMP2 or RAMP3 gives CLR a high affinity for AM, forming the AM receptor 1 and the AM receptor 2, respectively.

The three RAMPs are each composed of about 160 amino acids, and all exhibit a common structure that includes a large extracellular N-terminal domain (ECD), a single transmembrane domain (TM), and a very short cytoplasmic C-terminal tail (C-tail); however, they share less than 30% sequence homology. Although RAMPs are ubiquitous throughout the body, there are differences in their tissue distributions, and the abundance of each isoform depends on the tissue type. In addition, RAMP gene expression is differentially regulated under various disease conditions in animal models.

The collective findings brought together by the inventors and other researchers over the last years place AM as a major regulator of carcinogenesis-tumor progression in a number of cancers, including glioblastoma, prostate, colon, lung and mesothelioma, pheochromocytomas and renal carcinomas. In the light of our results, new therapeutic protocols and trials based on AM inhibition could be envisioned in cancer patients, which remain a major clinical concern.

However, to date, antibodies which may be used to inhibit the effects of adrenomedullin have eluded the field.

### SUMMARY OF THE INVENTION

Described herein is the generation of monoclonal antibodies that specifically bind to RAMP2 or RAMP3 and inhibit tumourigenic effects of adrenomedullin. These antibodies may therefore represent a therapeutic regimen in the field of cancer and angiogenesis.

As described herein, the present inventors have developed some monoclonal antibodies with specificity for RAMP2 and RAMP3 which not only potently bind the protein but also inhibit tumourigenic effects of adrenomedullin such as the pro-angiogenic effects and pro-invasive effect on cancer cells. This is the first demonstration of RAMP 2 and RAMP3 antibodies directly inhibiting the pro-tumourigenic effects of adrenomedullin and thus enables the use of such antibodies as active therapeutic agents with high specificity and low toxicity in a wide range of applications including cancer therapeutics.

Accordingly, in a first aspect, the present invention provides an antibody molecule which binds RAMP2 or RAMP3 and inhibits adrenomedullin activity.

In one embodiment, the antibody molecule is considered to inhibit adrenomedullin activity if it inhibits one or more of the following: (i) angiogenesis, (ii) tumour invasion, for example endothelial tumour invasion, and (iii) chemotaxis.

As described in the examples, antibody molecules of the invention have been shown to cause significant amounts of necrosis in tumours. Accordingly, in one embodiment of the invention, the antibody molecule of the invention induces or accelerates necrosis in the tumour vasculature or elsewhere in the tumour.

In one embodiment, the antibody molecule selectively binds RAMP2 or RAMP3 without significantly binding other polypeptides in the body.

In one embodiment, the antibody molecule binds the extracellular domain of RAMP2 or RAMP3.

Accordingly, in one embodiment of the invention, the antibody molecule binds an epitope on the extracellular domain of RAMP2 or RAMP3.

In one embodiment, the antibody molecule has an affinity for its target of at least 10-7 M, for example at least 10-8 M, or higher e.g. 10-9 M or higher.

In one embodiment, the antibody molecule comprises (i) an antigen binding domain comprising at least one, for example two or three, of the CDRs of the VH chain having the amino acid sequence shown as Seq ID No: 1 and/or (ii) an antigen binding domain comprising at least one, for example two or three, of the CDRs of the VL chain having the amino acid sequence shown as Seq ID No: 2.

In another embodiment, the antibody molecule comprises (i) an antigen binding domain comprising at least one, for example two or three, of the CDRs of the VH chain having the amino acid sequence shown as Seq ID No: 33 and/or (ii) an antigen binding domain comprising at least one, for example two or three, of the CDRs of the VL chain having the amino acid sequence shown as Seq ID No: 34.

In another embodiment, the antibody molecule comprises (i) an antigen binding domain comprising at least one, for example two or three, of the CDRs of the VH chain having the amino acid sequence shown as Seq ID No: 37 and/or (ii) an antigen binding domain comprising at least one, for example two or three, of the CDRs of the VL chain having the amino acid sequence shown as Seq ID No: 38.

In another embodiment, the antibody molecule comprises (i) an antigen binding domain comprising at least one, for example two or three, of the CDRs of the VH chain having the amino acid sequence shown as Seq ID No: 49 and/or (ii) an antigen binding domain comprising at least one, for example two or three, of the CDRs of the VL chain having the amino acid sequence shown as Seq ID No: 50.

CDRs may be identified using any suitable system. For example, the positions of such CDRs may be determined as described in Kabat et al, Sequences of Proteins of Immunological Interest, US Dept of Health and Human Services, Public Health Service, Nat'l Inst. of Health, NIH Publication No. 91-3242, 1991 and online at www.kabatdatabase.com http://immuno.bme.nwu.edu. An alternative system for determining CDRs which may be used is the IMGT unique numbering system (Lefranc M.-P. et al. “IMGT unique numbering for immunoglobulin and T cell receptor variable domains and Ig superfamily V-like domains” Dev. Comp. Immunol., 27, 55-77 (2003); Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008)). A further variable region numbering system which may be used is the Chothia system (Chothia C and Lesk A M, J. Mol. Biol. 1987, 196, p901; Al-Lazikani et al., (1997) JMB 273,927-948).

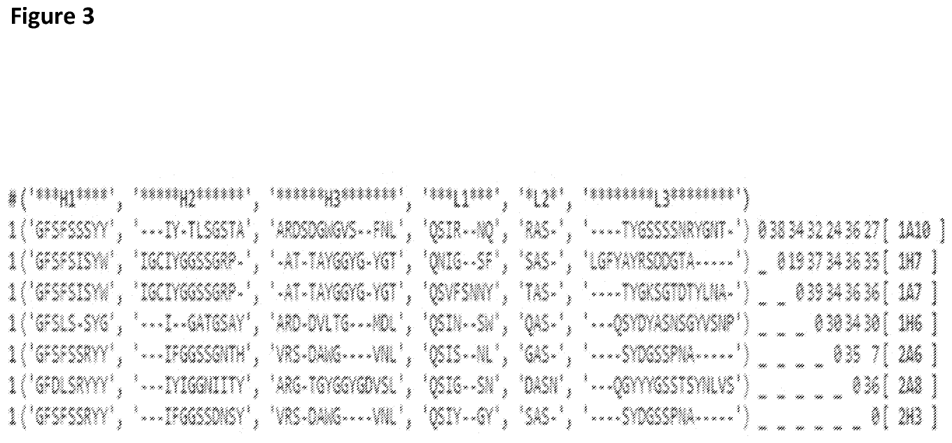

The VH and VL chains having the amino acid sequences shown as Seq ID No: 1 and Seq ID No: 2 respectively are the VL and VH chains of the 1H6 antibody. In one embodiment, CDRs of the VH and VL chains of this antibody are as shown in FIG. 3.

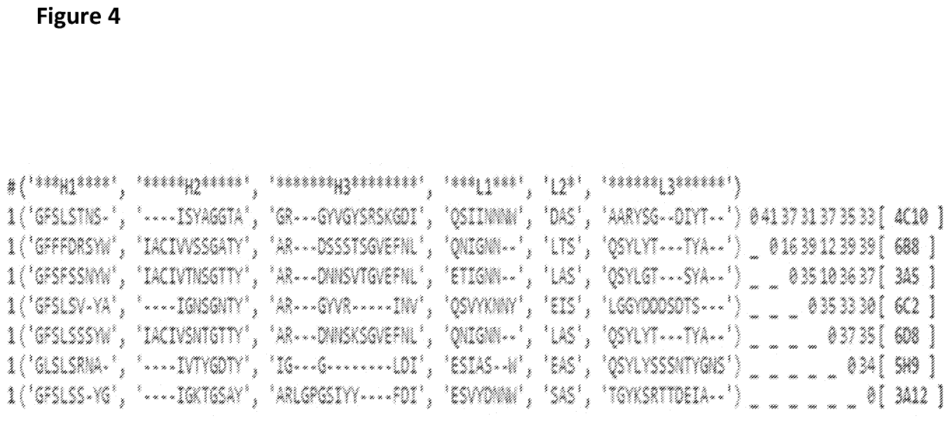

The VH and VL chains having the amino acid sequences shown as Seq ID No: 33 and Seq ID No: 34 respectively are the VL and VH chains of the 3A12 antibody. In one embodiment, CDRs of the VH and VL chains of this antibody are as shown in FIG. 4.

The VH and VL chains having the amino acid sequences shown as Seq ID No: 37 and Seq ID No: 38 respectively are the VL and VH chains of the 4C10 antibody. In one embodiment, CDRs of the VH and VL chains of this antibody are as shown in FIG. 4.

The VH and VL chains having the amino acid sequences shown as Seq ID No: 49 and Seq ID No: 50 respectively are the VL and VH chains of the 6C2 antibody. In one embodiment, CDRs of the VH and VL chains of this antibody are as shown in FIG. 4.

In one embodiment, the antibody molecule comprises (i) an antigen binding domain comprising all three of the CDRs of the VL chain having the amino acid sequence shown as Seq ID No: 1, or variants of said CDRs, and/or (ii) an antigen binding domain comprising at all three, of the CDRs of the VH chain having the amino acid sequence shown as Seq ID No: 2, or variants of said CDRs.

In another embodiment, the antibody molecule comprises (i) an antigen binding domain comprising all three of the CDRs of the VL chain having the amino acid sequence shown as Seq ID No: 33, or variants of said CDRs, and/or (ii) an antigen binding domain comprising all three of the CDRs of the VH chain having the amino acid sequence shown as Seq ID No: 34, or variants of said CDRs.

In another embodiment, the antibody molecule comprises (i) an antigen binding domain comprising all three of the CDRs of the VL chain having the amino acid sequence shown as Seq ID No: 37, or variants of said CDRs, and/or (ii) an antigen binding domain comprising all three of the CDRs of the VH chain having the amino acid sequence shown as Seq ID No: 38, or variants of said CDRs.

In another embodiment, the antibody molecule comprises (i) an antigen binding domain comprising all three of the CDRs of the VL chain having the amino acid sequence shown as Seq ID No: 49, or variants of said CDRs and/or (ii) an antigen binding domain comprising all three of the VH chain having the amino acid sequence shown as Seq ID No: 50, or variants of said CDRs.

In one embodiment, the antibody molecule comprises (i) an antigen binding domain comprising all three of the CDRs of the VL chain having the amino acid sequence shown as Seq ID No: 1, and (ii) an antigen binding domain comprising at all three, of the CDRs of the VH chain having the amino acid sequence shown as Seq ID No: 2.

In another embodiment, the antibody molecule comprises (i) an antigen binding domain comprising all three of the CDRs of the VL chain having the amino acid sequence shown as Seq ID No: 33, and (ii) an antigen binding domain comprising all three of the CDRs of the VH chain having the amino acid sequence shown as Seq ID No: 34.

In another embodiment, the antibody molecule comprises (i) an antigen binding domain comprising all three of the CDRs of the VL chain having the amino acid sequence shown as Seq ID No: 37, and (ii) an antigen binding domain comprising all three of the CDRs of the VH chain having the amino acid sequence shown as Seq ID No: 38.

In another embodiment, the antibody molecule comprises (i) an antigen binding domain comprising all three of the CDRs of the VL chain having the amino acid sequence shown as Seq ID No: 49, and (ii) an antigen binding domain comprising all three of the VH chain having the amino acid sequence shown as Seq ID No: 50.

In one embodiment, the antibody molecule comprises an antibody V_{L} domain or an antibody V_{H} domain, or both.

In a preferred embodiment, the antibody V_{L} domain comprises the amino acid sequence Seq ID No: 1 and/or the antibody V_{H} domain comprises the amino acid sequence Seq ID No: 2.

In another particular embodiment, the antibody V_{L} domain comprises the amino acid sequence Seq ID No: 33 and/or the antibody V_{H} domain comprises the amino acid sequence Seq ID No: 31.

In another embodiment, the antibody V_{L} domain consists of the amino acid sequence Seq ID No: 37 and/or the antibody V_{H} domain consists of the amino acid sequence Seq ID No: 38.

In another embodiment, the antibody V_{L} domain consists of the amino acid sequence Seq ID No: 49 and/or the antibody V_{H} domain consists of the amino acid sequence Seq ID No: 50.

The antibody molecule may be an antibody, for example a whole antibody.

In one alternative embodiment, the antibody molecule may be an antibody fragment such as an scFv.

The provision of the antibody molecule of the present invention enables the development of related antibodies which also inhibit adrenomedullin activity, for example pro-angiogenic activity or pro-invasive activity, and which optionally have similar or greater binding specificity.

Accordingly, further encompassed within the scope of the present invention are antibody molecules comprising at least one, for example two or three, of the CDRs of the VL chain having the amino acid sequence shown as Seq ID No: 1 and/or at least one, for example two or three, of the CDRs of the VH chain having the amino acid sequence shown as Seq ID No: 2, in which 5 or less, for example 4, 3, 2, or 1 amino acid substitutions, have been made in at least one of said CDRs and wherein the antibody molecule retains the ability to inhibit adrenomedullin activity, for example the pro-angiogenic activity or pro-invasive activity of adrenomedullin.

Likewise, the invention further encompasses antibody molecules comprising at least one, for example two or three, of the CDRs of a VL chain and/or at least one, for example two or three, of the CDRs of a VH chain, in which 5 or less, for example 4, 3, 2, or 1 amino acid substitutions, have been made in at least one of said CDRs and wherein the antibody molecule retains the ability to inhibit adrenomedullin activity, for example the pro-angiogenic activity or pro-invasive activity of adrenomedullin; wherein
(i) said VL chain has the amino acid sequence shown as Seq ID No: 33 and said VH chain has the amino acid sequence shown as Seq ID No: 34; or
(ii) said VL chain has the amino acid sequence shown as Seq ID No: 37 and said VH chain has the amino acid sequence shown as Seq ID No: 38; or
(iii) said VL chain has the amino acid sequence shown as Seq ID No: 45 and said VH chain has the amino acid sequence shown as Seq ID No: 46; or
(iv) said VL chain has the amino acid sequence shown as Seq ID No: 49 and said VH chain has the amino acid sequence shown as Seq ID No: 50; or
(v) said VL chain has the amino acid sequence shown as Seq ID No: 53 and said VH chain has the amino acid sequence shown as Seq ID No: 54.

In an embodiment of the first aspect of the invention, the antibody molecule has the ability to inhibit tumour cell invasion.

In another embodiment, the antibody molecule of the first aspect of the invention has the ability to inhibit angiogenesis.

In a second aspect of the invention, there is provided a nucleic acid encoding an antibody molecule according to the first aspect of the invention.

In one embodiment of the second aspect of the invention, the nucleic acid comprises
(i) the nucleotide sequence shown as VL 1H6 (Sequence ID No: 3), and/or the nucleotide sequence shown as VH 1H6 (Sequence ID No: 4); or
(ii) the nucleotide sequence shown as VL 3A12 (Sequence ID No: 35), and/or the nucleotide sequence shown as VH 3A12 (Sequence ID No: 36); or
(iii) the nucleotide sequence shown as VL 4C10 (Sequence ID No: 39), and/or the nucleotide sequence shown as VH 4C10 (Sequence ID No: 40); or
(iv) the nucleotide sequence shown as VL 6B8 (Sequence ID No: 47), and/or the nucleotide sequence shown as VH 6B8 (Sequence ID No: 48)
(v) the nucleotide sequence shown as VL 6C2 (Sequence ID No: 51), and/or the nucleotide sequence shown as VH 6C2 (Sequence ID No: 52); or
(vi) the nucleotide sequence shown as VL 6D8 (Sequence ID No: 55), and/or the nucleotide sequence shown as VH 6D8 (Sequence ID No: 56).

The nucleic acid may be used to provide antibody molecules according to the first aspect of the invention. Accordingly, there is provided a method of producing an antibody molecule capable of inhibiting the pro-tumourigenic activity of adrenomedullin, said method comprising expressing the nucleic acid according to the second aspect of the invention in a host cell and isolating said antibody molecule from said cell.

A further aspect of the invention is a pharmaceutical composition comprising an antibody molecule of the first aspect of the invention or a nucleic acid of the second aspect of the invention.

The antibody molecules, nucleic acids or compositions of the invention may be used for the inhibition of adrenomedullin activity, for example pro-angiogenic or pro-invasive activity, in particular where the adrenomedullin receptor is aberrantly expressed.

Accordingly, in a further aspect, the present invention provides a method of inhibiting adrenomedullin activity in a biological sample, said method comprising administration of an antibody molecule according to the first aspect of the present invention or a nucleic acid according to the second aspect of the invention to said biological sample.

In a further aspect, there is provided a method of treating a condition associated with activity of adrenomedullin in a patient in need of treatment thereof, said method comprising administration to said patient of an antibody molecule according to the first aspect of the present invention or a nucleic acid according to the second aspect of the invention.

In one embodiment, the condition is a condition associated with aberrant activity of adrenomedullin.

In the context of the present application, an adrenomedullin receptor, e.g. RAMP2 or RAMP3, is considered to be aberrantly expressed where its expression differs from that of normal healthy cells, for example expression which is greater than normal from or at a cell or tissue and where its aberrant expression contributes to a disease state.

Further provided is an antibody molecule according to the first aspect of the invention or a nucleic acid according to the second aspect of the invention for use in medicine.

The invention further provides an antibody molecule according to the first aspect of the invention or a nucleic acid according to the second aspect of the invention for use in treatment of a condition associated with aberrant adrenomedullin activity

Also provided is the use of an antibody molecule according to the first aspect of the invention or a nucleic acid according to the second aspect of the invention in the preparation of a medicament for the treatment of a condition associated with aberrant activity or expression of adrenomedullin.

The invention may be used in the treatment of any condition with which aberrant activity of adrenomedullin is associated. For example, conditions in which the invention may be used include, but are not limited to, diseases associated with excessive, deregulated or inappropriate angiogenesis and/or cancer.

In particular embodiments of the invention, the cancer is glioblastoma, renal cancer, prostate cancer, colon cancer, lung cancer, mesothelioma, or pheochromocytoma.

In one particular embodiment, the cancer is glioblastoma.

In another particular embodiment, the cancer is renal cancer.

### DETAILED DESCRIPTION

### Antibody Molecules

In the context of the present invention, an “antibody molecule” should be understood to refer to an immunoglobulin or part thereof or any polypeptide comprising a binding domain which is, or is homologous to, an antibody binding domain. Antibody molecules include but are not limited to polyclonal, monoclonal, monospecific, polyspecific antibodies and fragments thereof and chimeric antibodies comprising an immunoglobulin binding domain fused to another polypeptide.

Intact (whole) antibodies comprise an immunoglobulin molecule consisting of heavy chains and light chains, each of which carries a variable region designated VH and VL, respectively. The variable region consists of three complementarity determining regions (CDRs, also known as hypervariable regions) and four framework regions (FR) or scaffolds. The CDR forms a complementary steric structure with the antigen molecule and determines the specificity of the antibody.

Fragments of antibodies may retain the binding ability of the intact antibody and may be used in place of the intact antibody. Accordingly, for the purposes of the present invention, unless the context demands otherwise, the term “antibody molecules” should be understood to encompass antibody fragments. Examples of antibody fragments include Fab, Fab′, F(ab′)2, Fd, dAb, and Fv fragments, scFvs, bispecific scFvs, diabodies, linear antibodies (see US patent 5, 641, 870, Example 2; Zapata et al., Protein Eng 8(10): 1057-1062 [1995]); single-chain antibody molecules; and multispecific antibodies formed from antibody fragments.

The Fab fragment consists of an entire L chain (VL and CL), together with VH and CH1. Fab′ fragments differ from Fab fragments by having additional few residues at the carboxy terminus of the CH1 domain including one or more cysteines from the antibody hinge region. The F(ab′)2 fragment comprises two disulfide linked Fab fragments.

Fd fragments consist of the VH and CH1 domains.

Fv fragments consist of the VL and VH domains of a single antibody.

Single-chain Fv fragments are antibody fragments that comprise the VH and VL domains connected by a linker which enables the scFv to form an antigen binding site. (see Pluckthun in The Pharmacology of Monoclonal Antibodies, vol. 113, Rosenburg and Moore eds., Springer-Verlag, New York, pp. 269-315 (1994)).

Diabodies are small antibody fragments prepared by constructing scFv fragments with short linkers (about 5-10 residues) between the VH and VL domains such that inter-chain but not intra-chain pairing of the V domains is achieved, resulting in a multivalent fragment, i.e. a fragment having two antigen-binding sites (see, for example, EP 404 097; WO 93/11161; and Hollinger et al., Proc. Natl. Acad. Sci. USA, 90:6444-6448 (1993)).

In one embodiment of the invention, the antibody molecule is a bispecific antibody molecule, for example a bispecific antibody molecule having a first binding site with specificity for RAMP2 or RAMP3 and a second binding site with specificity for a second different antigen. In one embodiment, the second binding site has specificity for a T cell antigen, for example CD3. Such T cell engager bispecific antibodies have been described by Baeuerle et al, Drugs of the Future 33:137-147, Kufer et al Trends Biotechnol. 22:238-244 (2004) and Wolf et al, Drug Discovery Today 10:1237-44 (2005). An example of such an antibody molecule is the BiTE® antibody (Amgen, US), which binds cytotoxic T cells and target cells, bringing them together such that the T cell can induce lysis of the target cell with granzymes. In one embodiment the antibody molecule of the invention is a bispecific antibody molecule having a first binding site with specificity for RAMP2 or RAMP3 and a second binding site with specificity for VEGF (vascular endothelial growth factor) or EGFR (epidermal growth factor receptor).

Further encompassed by fragments are individual CDRs.

In the present invention, the amino acid sequences of the VH and VL regions of the intact anti RAMP2 antibodies rlgG-k1-1H6, rlgG-k1-1A7, rlgG-k1-1H7, rlgG-k1-1A10, rlgG-k1-2A6, rlgG-k1-2A8, and rlgG-k1-2H3have been identified. The inventors have also identified the amino acid sequences of the VH and VL regions of the intact anti RAMP3 antibodies rlgG-k1-3A5, rlgG-k1-3A12, rlgG-k1-4C10, rlgG-k1-5H9, rlgG-k1-6B8, rlgG-k1-6C2, and rlgG-k1-6D8. The present invention encompasses antibody molecules having the VH and/or VL sequences of these antibodies.

The amino acid sequences of these VH and VL regions are shown below, together with the nucleic acid sequence encoding the VH and VL regions.

Anti-RAMP2 Antibodies
RAMP2-rIgG-k1-1H6#4
VL:
(SEQ ID NO: 1)
APVLTQTPASVEAAVGGTVTIKCQASQSINSWLSWYQQKPGQPPKLLIYQASKLASGVPSRFKGSGSGTEYTLTISD
LKCADAATYYCQSYDYASNSGYVSNPFGGGTEVVVK
(SEQ ID NO: 3)
GCACCTGTGCTGACCCAGACACCAGCCTCCGTGGAGGCAGCTGTGGGAGGCACAGTCACCATCAAGTGCCAG
GCCAGTCAGAGTATTAATAGTTGGTTATCCTGGTATCAGCAGAAACCAGGGCAGCCTCCCAAGCTCCTGATCT
ACCAGGCATCCAAACTGGCATCTGGGGTCCCATCGCGGTTCAAAGGCAGTGGATCTGGGACAGAGTACACTC
TCACCATCAGCGACCTGAAGTGTGCCGATGCTGCCACTTATTATTGTCAAAGCTATGATTATGCTAGTAATAGT
GGATATGTGTCTAATCCTTTCGGCGGAGGGACCGAGGTGGTGGTCAAA
VH:
(SEQ ID NO: 2)
QEHLVESEGGLFKPTDTLTLTCTVSGFSLSSYGVSWVRQAPGKGLEWIGVIGATGSAYYANWAKSRSTITRNTNLN
TVTLKVTSLTAADTATYFCARDDVLTGMDLWGPGTLVTVSS
(SEQ ID NO: 4)
CAGGAGCATCTGGTGGAGTCCGAGGGAGGTCTCTTCAAGCCAACGGATACCCTGACACTCACCTGCACAGTCT
CTGGATTCTCCCTCAGTAGCTATGGAGTGAGCTGGGTCCGCCAGGCTCCAGGGAAGGGGCTGGAATGGATCG
GAGTCATTGGTGCTACTGGCAGCGCATACTACGCGAACTGGGCGAAAAGCCGATCCACCATCACCAGAAACA
CCAACCTGAACACAGTGACTCTGAAGGTGACCAGTCTGACAGCCGCGGACACGGCCACCTATTTCTGTGCGAG
GGATGATGTGCTTACTGGTATGGACCTCTGGGGCCCAGGGACCCTCGTCACCGTCTCTTCA
RAMP2-rIgG-k1-1A7#1
VL:
(SEQ ID NO: 5)
DGVMTQTPSSVSAAVGSTVTISCQASQSVFSNNYLSWYQQKPGQPPKLLIYTASSLASGVPSRFKGSGSGTQFTLTI
SDLECADAATYYCQCTYGKSGTDTYLNAFGGGTEVVVK
(SEQ ID NO: 7)
GATGGCGTGATGACCCAGACTCCATCCTCCGTGTCTGCAGCTGTGGGAAGCACAGTCACCATCAGTTGCCAGG
CCAGTCAGAGTGTTTTTAGTAACAACTACTTATCCTGGTATCAGCAGAAACCAGGGCAGCCTCCCAAGCTCCTG
ATCTATACTGCATCCAGTCTGGCATCTGGGGTCCCATCGCGGTTCAAAGGCAGTGGATCTGGGACACAGTTCA
CTCTCACCATCAGCGACCTGGAGTGTGCCGATGCTGCCACTTACTACTGTCAATGCACTTATGGAAAGAGTGG
TACTGATACTTATCTAAATGCTTTCGGCGGAGGGACCGAGGTGGTGGTCAAA
VH:
(SEQ ID NO: 6)
QSVEESGGDLVKPEGSLTLTCTASGFSFSISYWICWVRQTPGKGLEWIGCIYGGSSGRPYYASWAKGRFTISKTSSTT
VTLQMTSLTAADTAAYFCATTAYGGYGYGTWGPGTLVTVSS
(SEQ ID NO: 8)
CAGTCGGTGGAGGAGTCCGGGGGAGACCTGGTCAAGCCTGAGGGATCCCTGACACTCACCTGCACAGCCTCT
GGATTCTCCTTCAGTATCAGCTATTGGATATGCTGGGTCCGCCAGACTCCAGGGAAGGGGCTGGAGTGGATC
GGATGCATTTATGGTGGTAGTAGTGGTCGTCCTTACTACGCGAGCTGGGCGAAAGGCCGATTCACCATCTCCA
AAACCTCGTCGACCACGGTGACTCTGCAAATGACTAGTCTGACAGCCGCGGACACGGCCGCCTATTTCTGTGC
GACCACTGCTTATGGTGGTTATGGTTATGGTACGTGGGGCCCAGGCACCCTGGTCACCGTCTCCTCA
RAMP2-rIgG-k1-2A6
VL:
(SEQ ID NO: 9)
DVVMTQTPASVEAAVGGTVTIKCQASQSISNLLAWYQQKPGQPPKRLIYGASNLESGVPSRFKGSGSGTQFTLTIS
DLECADAATYYCQCSYDGSSPNAFGGGTEVVVE
(SEQ ID NO: 11)
GATGTTGTGATGACCCAGACTCCAGCCTCCGTGGAGGCAGCTGTGGGAGGCACAGTCACCATCAAGTGCCAG
GCCAGTCAGAGCATTAGCAACCTCTTAGCCTGGTATCAACAGAAACCAGGGCAGCCTCCCAAGCGCCTGATCT
ATGGTGCATCCAATCTGGAATCTGGGGTCCCATCGCGGTTCAAAGGCAGTGGATCTGGGACACAGTTCACTCT
CACCATCAGCGACCTGGAGTGTGCCGATGCTGCCACTTACTACTGTCAATGTAGTTATGATGGTAGTAGTCCC
AATGCTTTCGGCGGAGGGACCGAGGTGGTGGTCGAA
VH:
(SEQ ID NO: 10)
QSVEESGGDLVKPGASLTLTCTASGFSFSSRYYMCWVRQAPGKGLEWIGCIFGGSSGNTHYASWAKGRFTIAKTSS
TTVTLRMTSLTAADTATYSCVRSDAWGVNLWGPGTLVTVSS
(SEQ ID NO: 12)
CAGTCGGTGGAGGAGTCCGGGGGAGACCTGGTCAAGCCTGGGGCATCCCTGACACTCACCTGCACAGCCTCT
GGATTCTCCTTCAGTAGCCGCTACTATATGTGCTGGGTCCGCCAGGCTCCAGGGAAGGGGCTGGAGTGGATC
GGCTGTATTTTTGGTGGTAGTAGTGGTAATACTCACTACGCGAGCTGGGCGAAAGGCCGCTTCACCATCGCCA
AAACCTCGTCGACCACGGTGACTCTGCGAATGACCAGTCTGACAGCCGCGGACACGGCCACCTATTCTTGTGT
GAGAAGTGATGCCTGGGGTGTTAACTTGTGGGGCCCAGGCACCCTGGTCACCGTCTCCTCA
RAMP2-rIgG-k1-2A8
VL:
(SEQ ID NO: 13)
APVLTHTAASVSEPVGGIVTINCQASQSIGSNLAWYQQKSGQPPKLLIYDASNLVSGVPSRFKGSRSGTEYTLTISDL
ECADAGTYYCQGYYYGSSTSYNLVSFGGGTEVVVK
(SEQ ID NO: 15)
GCACCTGTGCTGACCCATACTGCAGCCTCCGTGTCTGAACCTGTGGGAGGCATAGTCACCATCAACTGCCAGG
CCAGTCAGAGCATTGGTAGTAATTTAGCCTGGTATCAGCAGAAATCAGGGCAGCCTCCCAAGCTCCTGATCTA
TGATGCATCCAATCTGGTATCTGGGGTCCCATCGCGGTTCAAAGGCAGCAGATCTGGGACAGAGTACACTCTC
ACCATCAGCGACCTGGAGTGTGCCGATGCTGGCACTTACTACTGTCAAGGCTATTATTATGGTAGTAGTACTA
GCTATAATTTAGTGTCTTTCGGCGGAGGGACCGAGGTGGTGGTCAAA
VH:
(SEQ ID NO: 14)
QSLEESGGGLVQPEGSLTLTCTASGFDLSRYYYIFWVRQAPGKGLEWIGCIYIGGNIITYYASWAKGRFTISKTSSPAV
TLQMTSLTVADTATYFCARGTGYGGYGDVSLWGPGTLVTVSS
(SEQ ID NO: 16)
CAGTCGTTGGAGGAGTCCGGGGGAGGCCTGGTCCAGCCTGAGGGATCCCTGACACTCACCTGCACAGCTTCT
GGATTCGACCTCAGTCGCTACTACTACATATTTTGGGTCCGCCAGGCTCCAGGGAAGGGGCTGGAGTGGATC
GGATGCATTTATATTGGTGGTAATATTATCACTTACTACGCGAGCTGGGCGAAAGGCCGCTTCACCATCTCCAA
AACCTCGTCGCCCGCGGTGACTCTGCAAATGACCAGCCTGACAGTCGCGGACACGGCCACCTATTTCTGTGCG
AGAGGGACTGGTTATGGTGGTTATGGTGATGTTAGCTTGTGGGGCCCAGGCACCCTGGTCACCGTCTCCTCA
RAMP2-rIgG-k1-2H3
VL:
(SEQ ID NO: 17)
AAVMTQTPSPVSGVVGGTVTIKCQASQSIYGYFSWYQQKPGQPPKLLIYSASDLASGVPSRFKGSGSGTQFTLTISD
LECADAATYYCQCSYDGSSPNAFGGGTEVVVK
(SEQ ID NO: 19)
GCAGCCGTGATGACCCAGACTCCATCCCCCGTGTCTGGAGTTGTGGGAGGCACAGTCACCATCAAGTGCCAG
GCCAGTCAGAGCATTTACGGCTACTTTTCTTGGTATCAGCAGAAACCAGGGCAGCCTCCCAAGCTCCTGATCTA
TTCTGCATCCGATCTGGCATCTGGGGTCCCATCGCGGTTCAAAGGCAGTGGATCTGGGACACAGTTCACTCTC
ACCATCAGCGACCTGGAGTGTGCCGATGCTGCCACTTACTACTGTCAATGTAGTTATGATGGTAGTAGTCCCA
ATGCTTTCGGCGGAGGGACCGAGGTGGTGGTCAAA
VH:
(SEQ ID NO: 18)
QSVEESGGDLVKPGASLTLTCTASGFSFSSRYYMCWVRQAPGKGLEWIACIFGGSSDNSYYARWAKGRFTISKTSS
TTVTLRMTSLTAADTATYFCVRSDAWGVNLWGPGTLVTVSS
(SEQ ID NO: 20)
CAGTCGGTGGAGGAGTCCGGGGGAGACCTGGTCAAGCCTGGGGCATCCCTGACACTCACCTGCACGGCCTCT
GGATTCTCCTTCAGTAGCCGCTATTATATGTGCTGGGTCCGCCAGGCTCCAGGGAAGGGGCTGGAGTGGATC
GCATGTATTTTTGGTGGTAGTAGTGATAATAGTTATTACGCGAGGTGGGCGAAAGGCCGCTTCACCATCTCCA
AAACCTCGTCGACCACGGTGACTCTGCGAATGACCAGTCTGACGGCCGCGGACACGGCCACTTATTTCTGTGT
GAGAAGTGATGCCTGGGGTGTTAACTTGTGGGGCCCAGGCACCCTGGTCACCGTCTCCTCA
RAMP2-rIgG-k1-1H7
VL:
(SEQ ID NO: 21)
AAVLTQTPSSTSGPVGGTVTIKCQASQNIGSFLSWYQQKPGQPPKLLIYSASSLATGVPSRFSGSGSGTQFTLTISGV
QCDDAATYYCLGFYAYRSDDGTAFGGGTEVVVK
(SEQ ID NO: 23)
GCAGCCGTGCTGACCCAGACTCCATCCTCCACGTCTGGACCAGTGGGAGGCACAGTCACCATCAAATGCCAG
GCTAGTCAGAATATTGGTAGTTTCTTATCCTGGTATCAGCAGAAACCAGGGCAGCCTCCCAAGCTCCTGATCTA
TTCTGCATCCAGTCTGGCAACTGGGGTCCCATCGCGGTTCAGCGGCAGTGGATCTGGGACACAGTTCACTCTC
ACCATTAGCGGCGTGCAGTGTGACGATGCTGCCACTTACTACTGTCTAGGTTTTTATGCTTATAGGAGTGATG
ACGGTACGGCTTTCGGCGGAGGGACCGAAGTGGTGGTCAAA
VH:
(SEQ ID NO: 22)
QSLEESGGDLVKPEGSLTLTCTASGFSFSISYWICWVRQTPGKGLEWIGCIYGGSSGRPYYASWAKGRFTISKTSSTT
VTLQMTSLTAADTAAYFCATTAYGGYGYGTWGPGTLVTVSS
(SEQ ID NO: 24)
CAGTCGTTGGAGGAGTCCGGGGGAGACCTGGTCAAGCCTGAGGGATCCCTGACACTCACCTGCACAGCCTCT
GGATTCTCCTTCAGTATCAGCTATTGGATATGCTGGGTCCGCCAGACTCCAGGGAAGGGGCTGGAGTGGATC
GGATGCATTTATGGTGGTAGTAGTGGTCGTCCTTACTACGCGAGCTGGGCGAAAGGCCGATTCACCATCTCCA
AAACCTCGTCGACCACGGTGACTCTGCAAATGACTAGTCTGACAGCCGCGGACACGGCCGCCTATTTCTGTGC
GACCACTGCTTATGGTGGTTATGGTTATGGTACGTGGGGCCCAGGCACCCTGGTCACCGTCTCCTCA
RAMP2-rIgG-k1-1A10
VL:
(SEQ ID NO: 25)
AAVMTQTPSSLSAAVGGTVTIKCQASQSIRNQVSWYQQKPGQRPKLLIYRASTLESGVPSRFKGSGSGTDFTLTISD
LECADAATYYCQCTYGSSSSNRYGNTFGGGTEVAVK
(SEQ ID NO: 27)
GCAGCCGTGATGACCCAGACTCCATCCTCCCTGTCTGCCGCTGTGGGAGGCACAGTCACCATCAAGTGCCAGG
CCAGTCAGAGTATTAGGAATCAAGTATCCTGGTATCAGCAGAAACCAGGGCAGCGTCCCAAGCTCCTGATCTA
CAGGGCATCCACTCTGGAATCTGGGGTCCCATCGCGGTTCAAAGGCAGTGGATCTGGGACAGACTTCACTCTC
ACCATCAGCGACCTGGAGTGTGCCGATGCTGCCACTTACTACTGTCAATGCACTTATGGTAGTAGTAGTAGTA
ATCGTTATGGAAATACTTTCGGCGGAGGGACCGAGGTGGCGGTCAAA
VH:
(SEQ ID NO: 26)
QSLEESGGGLVQPEGSLTLTCTASGFSFSSSYYMCWVRQAPGKGLEWIGSIYTLSGSTAYASWAKGRFTISKTSSTT
VTLQMTSLTAADTAMYFCARDSDGWGVSFNLWGPGTLVTVSS
(SEQ ID NO: 28)
CAGTCGTTGGAGGAGTCCGGGGGAGGCCTGGTCCAGCCTGAGGGATCCCTGACACTCACCTGCACAGCTTCT
GGATTCTCCTTCAGTAGCAGCTACTACATGTGCTGGGTCCGCCAGGCTCCAGGGAAGGGGCTGGAGTGGATC
GGATCCATTTATACTCTTAGTGGTAGCACTGCCTACGCGAGCTGGGCGAAAGGCCGATTCACCATCTCCAAAA
CCTCGTCGACCACGGTGACTCTGCAAATGACCAGTCTGACAGCCGCGGACACGGCCATGTATTTCTGTGCGAG
AGATAGTGATGGCTGGGGTGTCAGTITTAACTTGTGGGGCCCAGGCACCCTGGTCACCGTCTCCTCA
Anti-RAMP3 Antibodies
RAMP3-rIgG-k1-3A5
VL:
(SEQ ID NO: 29)
DVVMTQTPASVSAAVGATVTIKCQASETIGNNLAWYQQKPGQPPKVLMFLASTLASGVPSRFKGSRSGTEYTLTIS
GVQCDDAATYYCQSYLGTSYAFGGGTEVVVK
(SEQ ID NO: 31)
GATGTTGTGATGACCCAGACTCCAGCCTCCGTGTCTGCAGCTGTGGGAGCCACAGTCACCATCAAGTGCCAGG
CCAGTGAGACCATTGGTAATAATTTAGCCTGGTATCAGCAGAAACCAGGGCAGCCTCCCAAGGTCCTGATGTT
TCTGGCATCCACACTGGCATCTGGGGTCCCATCGCGGTTCAAAGGCAGTAGATCTGGGACAGAGTACACTCTC
ACCATCAGCGGCGTGCAGTGTGACGATGCTGCCACTTACTACTGTCAAAGCTATCTGGGTACTAGTTATGCTTT
CGGCGGAGGGACCGAGGTGGTCGTCAAA
VH:
(SEQ ID NO: 30)
QSLEESGGGLVQPEGTLTLTCTVSGFSFSSNYWICWVRQAPGKGLEWIACIVTNSGTTYYTSWAKGRLTLSKTSSTT
VTLQMTSLTAADTATYFCARDNNSVTGVEFNLWGPGTLVTVSS
(SEQ ID NO: 32)
CAGTCGTTGGAGGAGTCCGGGGGAGGCCTGGTCCAGCCTGAGGGAACCCTGACACTCACCTGCACAGTCTCT
GGATTCTCCTTCAGTAGCAACTACTGGATATGCTGGGTCCGCCAGGCTCCAGGGAAGGGGCTGGAGTGGATC
GCATGCATTGTTACTAATAGTGGTACTACTTACTACACGAGCTGGGCGAAAGGCCGACTCACCCTCTCCAAAA
CCTCATCGACCACGGTGACTCTACAAATGACCAGTCTGACAGCCGCGGACACGGCCACCTATTTCTGTGCGAG
AGATAATAATAGTGTGACTGGTGTTGAGTTTAATTTGTGGGGCCCAGGCACCCTGGTCACCGTCTCCTCA
RAMP3-rIgG-k1-3A12
VL:
(SEQ ID NO: 33)
DPMLTQTPSSKSVPVGGTVTINCQSSESVYDNNWLAWFQQKPGQPPKRLIYSASTLASGVSSRFKGSGSGTQFTLT
ISDVQCDDAATYYCTGYKSRTTDEIAFGGGTEVVVK
(SEQ ID NO: 35)
GACCCTATGCTGACCCAGACTCCATCTTCCAAGTCTGTCCCTGTGGGAGGCACAGTCACCATCAATTGCCAGTC
CAGTGAGAGTGTTTATGATAACAACTGGTTAGCCTGGTTTCAGCAGAAGCCAGGGCAGCCTCCCAAGCGCCT
GATCTATTCTGCATCCACTCTGGCATCTGGGGTCTCATCGCGATTCAAAGGCAGTGGATCTGGGACACAGTTC
ACTCTCACCATCAGCGACGTGCAGTGTGACGATGCTGCCACTTACTACTGTACAGGATATAAAAGCCGTACTA
CTGATGAGATTGCTTTCGGCGGAGGGACCGAGGTGGTGGTCAAA
VH:
(SEQ ID NO: 34)
QEQLEESEGGLFKPTDTLTLTCTVSGFSLSSYGVSWVRQAPGNGLEYIGFIGKTGSAYYASWAKSRSTITRNTTLNTV
TLKMTSLTVADTATYFCARLGPGSIYYFDIWGPGTLVTVSS
(SEQ ID NO: 36)
CAGGAGCAGCTGGAGGAGTCCGAGGGAGGTCTCTTCAAGCCAACGGATACCCTGACACTCACCTGCACAGTC
TCTGGATTCTCCCTCAGTAGCTATGGAGTGAGCTGGGTCCGCCAGGCTCCAGGGAACGGGCTGGAATACATC
GGATTCATTGGTAAAACTGGTAGCGCATACTACGCGAGCTGGGCGAAAAGCCGATCCACCATCACCAGAAAC
ACCACCCTGAACACGGTGACTCTGAAAATGACCAGTCTGACAGTCGCGGACACGGCCACCTATTTCTGTGCGA
GATTGGGACCTGGTAGTATTTACTATTTTGACATCTGGGGCCCAGGCACCCTGGTCACCGTCTCCTCA
RAMP3-rIgG-k1-4C10
VL:
(SEQ ID NO: 37)
DGVMTQTPSSTSAAVGSTVTISCQSTQSIINNNWLAWYQQKPGHSPKLLIYDASTLASGVPSRFSGSGSGTQFTLTI
SDLECDDAATYYCAARYSGDIYTFGGGTEVVVR
(SEQ ID NO: 39)
GATGGCGTGATGACCCAGACTCCATCTTCCACGTCTGCAGCTGTGGGAAGCACAGTCACCATCAGTTGCCAGT
CCACTCAGAGTATTATTAATAATAATTGGTTAGCCTGGTATCAGCAGAAACCAGGGCACTCTCCTAAGCTCCTG
ATCTACGATGCATCCACTCTGGCATCTGGGGTCCCATCACGGTTCAGCGGCAGTGGATCTGGGACACAGTTCA
CTCTCACCATCAGCGACCTGGAGTGTGACGATGCTGCCACTTACTACTGTGCAGCCCGTTATAGTGGTGATATT
TATACTTTCGGCGGAGGGACCGAGGTGGTGGTCAGA
VH:
(SEQ ID NO: 38)
QEQLEESGGGLVKPTDTLTLTCTVSGFSLSTNSIFWVRQAPGNGLEWIGAISYAGGTAYASWAKSRSTITRNTNLNT
VTLKMTSLTAADTATYFCGRGYVGYSRSKGDIWGPGTLVTVSS
(SEQ ID NO: 40)
CAGGAGCAGCTGGAGGAATCCGGGGGAGGCCTGGTCAAGCCAACGGATACCCTGACACTCACCTGCACAGT
CTCTGGATTCTCCCTCAGTACCAATTCAATTTTCTGGGTCCGCCAGGCTCCAGGGAACGGGCTGGAATGGATC
GGAGCCATTAGTTATGCTGGTGGCACAGCCTACGCGAGCTGGGCGAAAAGCCGATCCACCATCACCAGAAAC
ACCAACCTGAACACGGTGACTCTGAAGATGACTAGTCTGACAGCCGCGGACACGGCCACCTATTTCTGTGGGA
GAGGTTATGTTGGTTATAGTCGTAGTAAGGGGGATATCTGGGGCCCAGGCACCCTGGTCACCGTCTCCTCA
RAMP3-rIgG-k1-5H9
VL:
(SEQ ID NO: 41)
DVVMTQTPASVEAAVGGTVTIKCQATESIASWLAWYQQKAGQPPKLLIYEASKLESGVPSRFKGSGSGTEFTLTISD
LECADAATYYCQSYLYSSSNTYGNSFGGGTEVVVK
(SEQ ID NO: 43)
GATGTTGTGATGACCCAGACTCCAGCCTCCGTGGAGGCAGCTGTGGGAGGCACAGTCACCATCAAGTGCCAG
GCCACTGAGAGCATTGCCAGTTGGTTAGCCTGGTATCAGCAGAAAGCAGGGCAGCCTCCCAAGCTCCTGATCT
ACGAAGCATCCAAACTGGAATCTGGGGTCCCATCGCGGTTCAAAGGCAGTGGATCTGGGACAGAGTTCACTC
TCACCATCAGCGACCTGGAGTGTGCCGATGCTGCCACTTACTACTGTCAAAGCTATCTTTATAGTAGCAGTAAT
ACTTATGGTAATTCTTTCGGCGGAGGGACCGAGGTGGTGGTCAAA
VH:
(SEQ ID NO: 42)
QSLEESEGGLFKPTDTLTVTCIVSGLSLSRNAMSWVRQAPGSGLEWIGVIVTYGDTYYASWAKSRSTITRNTNENTV
TLKMTSLTAADTATYFCIGGLDIWGPGTLVTVSS
(SEQ ID NO: 44)
CAGTCGTTGGAGGAGTCCGAGGGAGGTCTCTTCAAGCCAACGGATACCCTGACAGTCACCTGTATAGTCTCTG
GACTCTCCCTCAGTAGGAATGCAATGAGCTGGGTCCGCCAGGCTCCAGGGAGCGGGCTGGAATGGATCGGA
GTCATTGTGACTTATGGTGACACATACTACGCGAGCTGGGCGAAAAGCCGATCCACCATCACCAGAAACACCA
ACGAGAACACGGTGACTCTGAAAATGACCAGTCTGACAGCCGCGGACACGGCCACCTATTTTTGTATTGGTGG
TCTTGACATCTGGGGCCCAGGCACCCTGGTCACCGTCTCCTCA
RAMP3-rIgG-k1-6B8
VL:
(SEQ ID NO: 45)
DVMMTQTPASVSEPVGGTVTIKCQASQNIGNNLAWYQQKPGQPPKLLIYLTSTLASGVPSRFKGSRSGTEFTLTIS
DLECADAATYFCQSYLYTTYAFGGGTEVVVK
(SEQ ID NO: 47)
GATGTCATGATGACCCAGACTCCAGCCTCCGTGTCTGAACCTGTGGGAGGCACAGTCACCATCAAGTGCCAGG
CCAGTCAGAACATTGGTAATAATTTAGCCTGGTATCAGCAGAAACCAGGGCAGCCTCCCAAGCTCCTGATCTA
TCTGACATCCACTCTGGCATCTGGGGTCCCATCGCGGTTCAAAGGCAGTAGATCTGGGACAGAGTTCACTCTC
ACCATCAGCGACCTGGAGTGTGCCGATGCTGCCACTTACTTCTGTCAAAGCTATTTATATACTACTTATGCTTTC
GGCGGAGGGACCGAGGTGGTCGTCAAA
VH:
(SEQ ID NO: 46)
QSLEESGGGLVKPEGSLTLTCTASGFFFDRSYWICWVRQTPAKGLEWIACIVVSSGATYYASWAKGRLTLSKTSSTT
VTLQMTSLTAADTAIYFCARDSSSTSGVEFNLWGPGTLVTVSS
(SEQ ID NO: 48)
CAGTCGTTGGAGGAGTCCGGGGGAGGCCTGGTCAAGCCTGAGGGATCCCTGACACTCACCTGCACAGCCTCT
GGTTTCTTCTTCGATAGAAGCTACTGGATATGCTGGGTCCGCCAGACTCCAGCAAAGGGGCTGGAGTGGATC
GCATGCATTGTTGTTAGCAGTGGTGCTACTTACTACGCGAGCTGGGCGAAAGGCCGACTCACCCTCTCCAAAA
CCTCGTCGACCACGGTGACTCTACAAATGACCAGTCTGACAGCCGCGGACACGGCCATCTATTTCTGTGCGAG
AGATAGTAGTAGTACTAGTGGTGTTGAGTTTAATTTGTGGGGCCCAGGCACCCTGGTCACCGTCTCCTCA
RAMP3-rIgG-k1-6C2
VL:
(SEQ ID NO: 49)
DGVMTQTPSSVSAAVGGTVTISCQSSQSVYKNNYLSWFQQKPGQPPKLLIYEISKLESGVPDRFSGSGSGTQFTLTI
SGVQCDDAATYYCLGGYDDDSDTSFGGGTEVVVK
(SEQ ID NO: 51)
GATGGCGTGATGACCCAGACTCCATCGTCCGTGTCTGCAGCTGTGGGAGGCACAGTCACCATCAGTTGCCAGT
CCAGTCAGAGTGTTTATAAGAACAACTACTTATCCTGGTTTCAGCAGAAACCAGGGCAGCCTCCCAAGCTCCT
GATCTACGAAATATCCAAACTGGAATCTGGGGTCCCAGATAGGTTCAGCGGCAGTGGATCTGGGACACAGTT
CACTCTCACCATCAGCGGCGTGCAGTGTGACGATGCTGCCACTTACTACTGTCTAGGCGGTTATGATGATGAT
TCTGATACTTCTTTCGGCGGAGGGACCGAGGTGGTGGTCAAA
VH:
(SEQ ID NO: 50)
QEQLEESEGGLFKPTDTLTLTCTVSGFSLSVYALTWVRQAPGNGLEWIGAIGNSGNTYYASWAKSRSTITRNTNEN
TVTLKMTSLTAADTATYFCARGYVRINVWGPGTLVTVSS
(SEQ ID NO: 52)
CAGGAGCAGCTGGAGGAGTCCGAGGGAGGTCTCTTCAAGCCAACGGATACCCTGACACTCACCTGCACAGTC
TCTGGATTCTCCCTCAGTGTCTATGCTTTAACCTGGGTCCGCCAGGCTCCAGGGAACGGGCTGGAGTGGATCG
GAGCCATTGGTAATAGTGGTAACACGTACTACGCGAGCTGGGCGAAAAGCCGATCCACCATCACCAGAAACA
CCAACGAGAACACGGTGACTCTGAAAATGACCAGTCTGACAGCCGCGGACACGGCCACCTATTTCTGTGCGA
GAGGATATGTCCGAATTAACGTTTGGGGCCCAGGCACCCTGGTCACCGTCTCCTCG
RAMP3-rIgG-k1-6D8
VL:
(SEQ ID NO: 53)
DVVMTQTPASVSEPVGGTVTIKCQASQNIGNNLAWYQQKPGHPPKLLIYLASTLASGVPSRFKGSRSGTEFTLTISD
LECADAATYYCQSYLYTTYAFGGGTEVVVK
(SEQ ID NO: 55)
GATGTTGTGATGACCCAGACTCCAGCCTCCGTGTCTGAACCTGTGGGAGGCACAGTCACCATCAAGTGCCAGG
CCAGTCAGAACATTGGTAATAATTTAGCCTGGTATCAGCAGAAACCAGGGCACCCTCCCAAGCTCCTGATCTA
TCTGGCATCTACTCTGGCATCTGGGGTCCCATCGCGATTCAAAGGCAGTAGATCTGGGACAGAGTTCACTCTC
ACCATCAGCGACCTGGAGTGTGCCGATGCTGCCACTTACTACTGTCAAAGCTATTTGTATACTACTTATGCTTT
CGGCGGAGGGACCGAGGTGGTCGTCAAA
VH:
(SEQ ID NO: 54)
QSVEESGGDLVRPEGSLTLTCTASGFSLSSSYWICWVRQAPGKGLEWIACIVSNTGTTYYASWAKGRLTLSKTSSTT
VTLQMTSLTAADTATYFCARDNNSKSGVEFNLWGPGTLVTVSS
(SEQ ID NO: 56)
CAGTCGGTGGAGGAGTCCGGGGGAGACCTGGTCAGGCCTGAGGGATCCCTGACACTCACCTGCACAGCTTCT
GGATTCTCCCTCAGTAGCAGCTACTGGATATGCTGGGTCCGCCAGGCTCCAGGAAAGGGGCTAGAGTGGATC
GCATGCATTGTTAGTAATACTGGTACCACTTACTACGCGAGCTGGGCGAAAGGCCGACTCACCCTCTCCAAAA
CCTCGTCGACCACGGTGACTCTGCAAATGACCAGTCTGACAGCCGCGGACACGGCCACCTATTTCTGTGCGAG
AGATAATAATAGTAAAAGTGGTGTTGAGTTTAATTTGTGGGGCCCAGGCACCCTGGTCACCGTCTCCTCA

As described above, the antibody molecule of the present invention is not limited to the specific sequences of the rlgG-k1-1H6, rlgG-k1-1A7, rlgG-k1-1H7, rlgG-k1-1A10, rlgG-k1-2A6, rlgG-k1-2A8, rlgG-k1-2H3, rlgG-k1-3A5, rlgG-k1-3A12, rlgG-k1-4C10, rlgG-k1-5H9, rlgG-k1-6B8, rlgG-k1-6C2, and rlgG-k1-6D8 antibodies, the VH, VL and the CDRs disclosed herein but also extends to variants thereof which maintain the ability to inhibit function or activity, of adrenomedullin, for example the pro-angiogenic or pro-invasive activity of adrenomedullin.

In a particular embodiment, the antibody molecule is a humanized version of a rlgG-k1-1H6, rlgG-k1-1A7, rlgG-k1-1H7, rlgG-k1-1A10, rlgG-k1-2A6, rlgG-k1-2A8, rlgG-k1-2H3, rlgG-k1- 3A5, rlgG-k1-3A12, rlgG-k1-4C10, rlgG-k1-5H9, rlgG-k1-6B8, rlgG-k1-6C2, or rlgG-k1-6D8 antibody.

In one embodiment, the invention encompasses an antibody molecule which comprises at least one, for example two or three, of the CDRs of a VL chain and/or at least one, for example two or three, of the CDRs of a VH chain, wherein the antibody molecule retains the ability to inhibit adrenomedullin activity, for example the pro-angiogenic activity or pro-invasive activity of adrenomedullin; and wherein:
(i) said VL chain has the amino acid sequence shown as Seq ID No: 1 and said VH chain has the amino acid sequence shown as Seq ID No: 2; or
(ii) said VL chain has the amino acid sequence shown as Seq ID No: 5 and said VH chain has the amino acid sequence shown as Seq ID No: 6; or
(iii) said VL chain has the amino acid sequence shown as Seq ID No: 9 and said VH chain has the amino acid sequence shown as Seq ID No: 10, or
(iv) said VL chain has the amino acid sequence shown as Seq ID No: 13 and said VH chain has the amino acid sequence shown as Seq ID No: 14; or
(v) said VL chain has the amino acid sequence shown as Seq ID No: 17 and said VH chain has the amino acid sequence shown as Seq ID No: 18; or
(vi) said VL chain has the amino acid sequence shown as Seq ID No: 21 and said VH chain has the amino acid sequence shown as Seq ID No: 22, or
(vii) said VL chain has the amino acid sequence shown as Seq ID No: 25 and said VH chain has the amino acid sequence shown as Seq ID No: 26; or
(viii) said VL chain has the amino acid sequence shown as Seq ID No: 29 and said VH chain has the amino acid sequence shown as Seq ID No: 30; or
(ix) said VL chain has the amino acid sequence shown as Seq ID No: 33 and said VH chain has the amino acid sequence shown as Seq ID No: 34, or
(x) said VL chain has the amino acid sequence shown as Seq ID No: 37 and said VH chain has the amino acid sequence shown as Seq ID No: 38; or
(xi) said VL chain has the amino acid sequence shown as Seq ID No: 41 and said VH chain has the amino acid sequence shown as Seq ID No: 42; or
(xii) said VL chain has the amino acid sequence shown as Seq ID No: 45 and said VH chain has the amino acid sequence shown as Seq ID No: 46; or
(xiii) said VL chain has the amino acid sequence shown as Seq ID No: 49 and said VH chain has the amino acid sequence shown as Seq ID No: 50; or
(xiv) said VL chain has the amino acid sequence shown as Seq ID No: 53 and said VH chain has the amino acid sequence shown as Seq ID No: 54.

In another embodiment, the invention encompasses an antibody molecule which comprises at least one, for example two or three, of the CDRs of a VL chain and at least one, for example, two or three, of the CDRs of a VH chain, wherein the antibody molecule retains the ability to inhibit adrenomedullin activity, for example the pro-angiogenic activity or pro-invasive activity of adrenomedullin; wherein:
(i) said VL chain has the amino acid sequence shown as Seq ID No: 1 and said VH chain has the amino acid sequence shown as Seq ID No: 2; or
(ii) said VL chain has the amino acid sequence shown as Seq ID No: 5 and said VH chain has the amino acid sequence shown as Seq ID No: 6; or
(iii) said VL chain has the amino acid sequence shown as Seq ID No: 9 and said VH chain has the amino acid sequence shown as Seq ID No: 10, or
(iv) said VL chain has the amino acid sequence shown as Seq ID No: 13 and said VH chain has the amino acid sequence shown as Seq ID No: 14; or
(v) said VL chain has the amino acid sequence shown as Seq ID No: 17 and said VH chain has the amino acid sequence shown as Seq ID No: 18; or
(vi) said VL chain has the amino acid sequence shown as Seq ID No: 21 and said VH chain has the amino acid sequence shown as Seq ID No: 22, or
(vii) said VL chain has the amino acid sequence shown as Seq ID No: 25 and said VH chain has the amino acid sequence shown as Seq ID No: 26; or
(viii) said VL chain has the amino acid sequence shown as Seq ID No: 29 and said VH chain has the amino acid sequence shown as Seq ID No: 30; or
(ix) said VL chain has the amino acid sequence shown as Seq ID No: 33 and said VH chain has the amino acid sequence shown as Seq ID No: 34, or
(x) said VL chain has the amino acid sequence shown as Seq ID No: 37 and said VH chain has the amino acid sequence shown as Seq ID No: 38; or
(xi) said VL chain has the amino acid sequence shown as Seq ID No: 41 and said VH chain has the amino acid sequence shown as Seq ID No: 42; or
(xii) said VL chain has the amino acid sequence shown as Seq ID No: 45 and said VH chain has the amino acid sequence shown as Seq ID No: 46; or
(xiii) said VL chain has the amino acid sequence shown as Seq ID No: 49 and said VH chain has the amino acid sequence shown as Seq ID No: 50; or
(xiv) said VL chain has the amino acid sequence shown as Seq ID No: 53 and said VH chain has the amino acid sequence shown as Seq ID No: 54.

In another embodiment, the invention encompasses an antibody molecule which comprises all three of the CDRs of a VL chain and all 3 three of the CDRs of a VH chain, wherein the antibody molecule retains the ability to inhibit adrenomedullin activity, for example the pro-angiogenic activity or pro-invasive activity of adrenomedullin; wherein
(i) said VL chain has the amino acid sequence shown as Seq ID No: 1 and said VH chain has the amino acid sequence shown as Seq ID No: 2; or
(ii) said VL chain has the amino acid sequence shown as Seq ID No: 5 and said VH chain has the amino acid sequence shown as Seq ID No: 6; or
(iii) said VL chain has the amino acid sequence shown as Seq ID No: 9 and said VH chain has the amino acid sequence shown as Seq ID No: 10, or
(iv) said VL chain has the amino acid sequence shown as Seq ID No: 13 and said VH chain has the amino acid sequence shown as Seq ID No: 14; or
(v) said VL chain has the amino acid sequence shown as Seq ID No: 17 and said VH chain has the amino acid sequence shown as Seq ID No: 18; or
(vi) said VL chain has the amino acid sequence shown as Seq ID No: 21 and said VH chain has the amino acid sequence shown as Seq ID No: 22, or
(vii) said VL chain has the amino acid sequence shown as Seq ID No: 25 and said VH chain has the amino acid sequence shown as Seq ID No: 26; or
(viii) said VL chain has the amino acid sequence shown as Seq ID No: 29 and said VH chain has the amino acid sequence shown as Seq ID No: 30; or
(ix) said VL chain has the amino acid sequence shown as Seq ID No: 33 and said VH chain has the amino acid sequence shown as Seq ID No: 34, or
(x) said VL chain has the amino acid sequence shown as Seq ID No: 37 and said VH chain has the amino acid sequence shown as Seq ID No: 38; or
(xi) said VL chain has the amino acid sequence shown as Seq ID No: 41 and said VH chain has the amino acid sequence shown as Seq ID No: 42; or
(xii) said VL chain has the amino acid sequence shown as Seq ID No: 45 and said VH chain has the amino acid sequence shown as Seq ID No: 46; or
(xiii) said VL chain has the amino acid sequence shown as Seq ID No: 49 and said VH chain has the amino acid sequence shown as Seq ID No: 50; or
(xiv) said VL chain has the amino acid sequence shown as Seq ID No: 53 and said VH chain has the amino acid sequence shown as Seq ID No: 54.

The CDR sequences of the rlgG-k1-1H6, rlgG-k1-1A7, rlgG-k1-1H7, rlgG-k1-1A10, rlgG-k1-2A6, rlgG-k1-2A8, rlgG-k1-2H3, rlgG-k1-3A5, rlgG-k1-3A12, rlgG-k1-4C10, rlgG-k1-5H9, rlgG-k1-6B8, rlgG-k1-6C2, and rlgG-k1-6D8 antibody molecules are shown in FIGS. 3 and 4.

The present invention also extends to variants of antibody molecules of the above embodiments in which one or more of the CDR sequences are modified. The modified amino acid residues in the amino acid sequences of the CDR variant are preferably 30% or less, more preferably 20% or less, most preferably 10% or less of the total number of amino acids of the entire CDR. Such variants may be provided using the teaching of the present application and techniques known in the art. The CDRs may be carried in a framework structure comprising an antibody heavy or light chain sequence or part thereof. Preferably such CDRs are positioned in a location corresponding to the position of the CDR(s) of naturally occurring VH and VL domains. The positions of such CDRs may be determined as described in Kabat et al, Sequences of Proteins of Immunological Interest, US Dept of Health and Human Services, Public Health Service, Nat'l Inst. of Health, NIH Publication No. 91-3242, 1991 and online at www.kabatdatabase.com http://immuno.bme.nwu.edu.

In one such embodiment, the antibody molecule comprises antibody molecules comprising at least one, for example two or three, of the CDRs of a VL chain and/or at least one, for example two or three, of the CDRs of a VH chain, wherein (a)
(i) said VL chain has the amino acid sequence shown as Seq ID No: 1 and said VH chain has the amino acid sequence shown as Seq ID No: 2;
(ii) said VL chain has the amino acid sequence shown as Seq ID No: 5 and said VH chain has the amino acid sequence shown as Seq ID No: 6;
(iii) said VL chain has the amino acid sequence shown as Seq ID No: 9 and said VH chain has the amino acid sequence shown as Seq ID No: 10,
(iv) said VL chain has the amino acid sequence shown as Seq ID No: 13 and said VH chain has the amino acid sequence shown as Seq ID No: 14;
(v) said VL chain has the amino acid sequence shown as Seq ID No: 17 and said VH chain has the amino acid sequence shown as Seq ID No: 18;
(vi) said VL chain has the amino acid sequence shown as Seq ID No: 21 and said VH chain has the amino acid sequence shown as Seq ID No: 22,
(vii) said VL chain has the amino acid sequence shown as Seq ID No: 25 and said VH chain has the amino acid sequence shown as Seq ID No: 26;
(viii) said VL chain has the amino acid sequence shown as Seq ID No: 29 and said VH chain has the amino acid sequence shown as Seq ID No: 30;
(ix) said VL chain has the amino acid sequence shown as Seq ID No: 33 and said VH chain has the amino acid sequence shown as Seq ID No: 34,
(x) said VL chain has the amino acid sequence shown as Seq ID No: 37 and said VH chain has the amino acid sequence shown as Seq ID No: 38;
(xi) said VL chain has the amino acid sequence shown as Seq ID No: 41 and said VH chain has the amino acid sequence shown as Seq ID No: 42; or
(xii) said VL chain has the amino acid sequence shown as Seq ID No: 45 and said VH chain has the amino acid sequence shown as Seq ID No: 46;
(xiii) said VL chain has the amino acid sequence shown as Seq ID No: 49 and said VH chain has the amino acid sequence shown as Seq ID No: 50; or
(xiv) said VL chain has the amino acid sequence shown as Seq ID No: 53 and said VH chain has the amino acid sequence shown as Seq ID No: 54;
and wherein (b) 5 or less, for example 4, 3, 2, or 1 amino acid substitutions, have been made in at least one of said CDRs and wherein the antibody molecule retains the ability to inhibit adrenomedullin activity, for example the pro-angiogenic activity or pro-invasive activity of adrenomedullin.

Furthermore, modifications may alternatively or additionally be made to the Framework Regions of the variable regions. Such changes in the framework regions may improve stability and reduce immunogenicity of the antibody.

The antibody molecules of the invention herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

In particular embodiments of the invention, the antibody molecule of and for use in the invention is a humanized antibody. Humanized antibody molecules for use in the present invention may be produced in any suitable way.

For example, in certain embodiments of the invention the antibody molecule is a humanized version of a rlgG-k1-6D8 antibody.

In one such embodiment, the humanised antibody has a heavy chain variable sequence selected from the VH humanized variants having the amino acid sequences shown as Sequence ID Nos: 65, 66, 67, 68, and 69, and/or a light chain variable sequence selected from the VL humanized variants having the amino acid sequences shown as Sequence ID Nos: 70, 71, 72, 73, and 74. These VH and VL sequences are shown below:

>VH1
(Sequence ID No: 65)

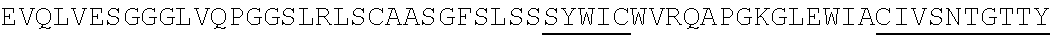

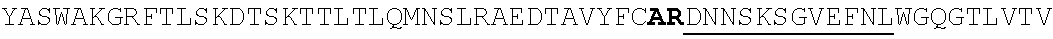

SS
>VH2
(Sequence ID No: 66)

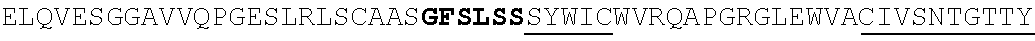

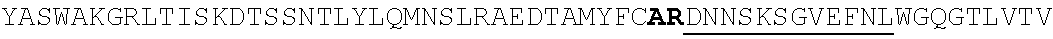

SS
>VH3
(Sequence ID No: 67)

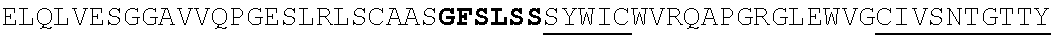

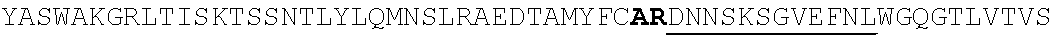

S
>VH4
(Sequence ID No: 68)

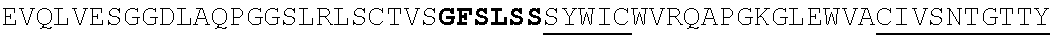

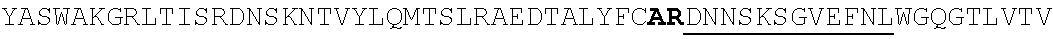

SS
>VH5
(Sequence ID No: 69)

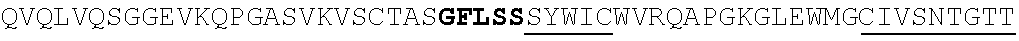

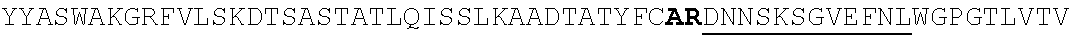

SS
VL domain Humanized variants:
>VL1
(Sequence ID No: 70)

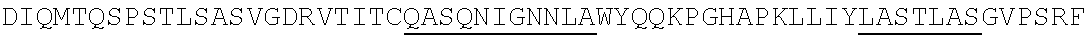

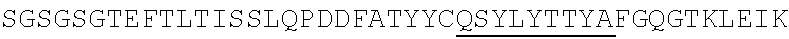

>VL2
(Sequence ID No: 71)

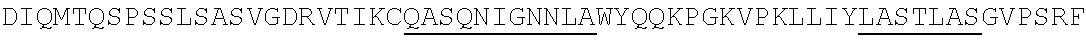

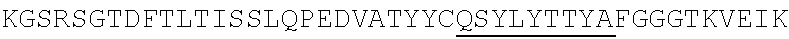

>VL3
(Sequence ID No: 72)

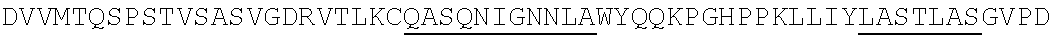

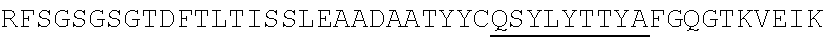

>VL4
(Sequence ID No: 73)

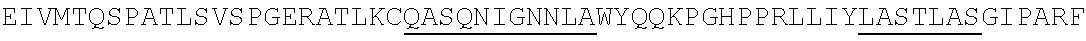

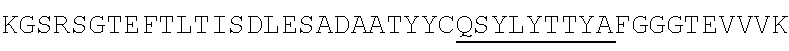

>VL5
(Sequence ID No: 74)

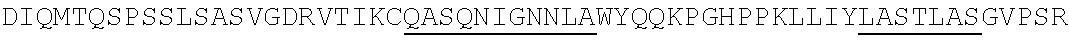

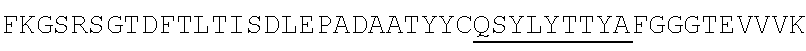

In one such embodiment, the the humanised antibody has a heavy chain variable sequence selected from the VH humanized variants having the amino acid sequences shown as Sequence ID Nos: 65, 66, 67, 68, and 69, and a light chain variable sequence selected from the VL humanized variants having the amino acid sequences shown as Sequence ID Nos: 70, 71, 72, 73, and 74.

A number of approaches have been developed to produce humanized antibodies and thus address the issue of unwanted immune responses being raised against non-human therapeutic antibodies which are administered to individuals. Typically, these approaches involve techniques which result in the replacement of certain components of the non-human antibody with equivalent portions derived from a human antibody. Such approaches can, for example, result in the production of chimeric antibodies which comprise non-human variable regions joined to human-derived constant regions.

Alternatively, antibodies may be humanized, using techniques such as “CDR grafting” or Composite Human Antibody Technology™, (Abzena, Cambridge, UK). Composite Human Antibodies are entirely human in origin and comprise multiple segments of human variable region sequence from different human antibodies. Such technology may thus be used to produce a fully humanized monoclonal antibody which displays a single binding specificity and which has variable regions in which both framework and CDR regions are derived from human germline immunoglobulin sequences. This contrasts with humanised antibodies produced by “CDR grafting”, wherein the complementarity determining regions (CDRs) from a murine antibody are grafted into a framework acceptor sequence, provided by regions of human antibody light and heavy chain variable domains. This results in the production of an antibody which retains the binding specificity of the murine antibody, but where the only non-human components are the grafted murine CDR regions.

With CDR grafting, the therapeutic effectiveness of the resulting humanised antibody may be impaired. For example, it has been observed that simple transplantation of CDR regions often results in a reduced therapeutic efficacy of the antibody due to the binding affinity of the antibody being diminished. To address this problem, back mutations may be employed, back mutations being the replacement of an amino acid residue at a specific position of the framework sequence (outside of the CDR sequence) so as to improve the binding specificity of the humanised antibody.

Typically in CDR grafting techniques, the CDRs of a donor antibody are selected using one of the Kabat, IMGT or Chothia methods and, typically, it is necessary to back-mutate certain residues outside the CDR sequences to restore satisfactory affinity. As described in WO2014/072741, humanised antibodies of high affinity may be readily obtained by using CDRs defined by a method which combines the IMGT and Kabat methods for defining CDR sequences.

Thus in embodiments of the invention, humanised antibody molecules may be employed in which the humanised antibody molecule has a heavy chain, wherein the complementarity determining regions (CDRs) are derived from a non-human donor antibody, wherein
(i) the first amino acid residue and the final amino acid residue of CDRH1 of the humanised antibody molecule are the first amino acid residue of CDRH1 of the donor antibody as defined by the IMGT method and the final amino acid residue as defined by the Kabat method respectively and the CDRH1 comprises the intervening amino acid residues of said donor CDRH1 between said first and final amino acid residues;
(ii) the first amino acid residue and the final amino acid residue of CDRH3 of the humanised antibody molecule are the first amino acid residue of CDRH3 of the donor antibody as defined by the IMGT method and the final amino acid residue as defined by the Kabat method respectively and the CDRH3 comprises the intervening amino acid residues of said donor CDRH3 between said first and final amino acid residues; and
(iii) CDRH2 of the humanised antibody molecule corresponds to CDRH2 of the donor antibody as defined by the Kabat method.

The term “derived” is intended to not only encompass the source of material as being the physical source but also to define material which is structurally identical to the material but which does not originate from the reference source. Thus the CDRs derived from a non-human donor antibody need not be necessarily purified or isolated from the donor antibody or antibody framework.

### Production of Antibody Molecules

Antibody molecules of and for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352:624-628 and Marks et al. (1992) Bio/Technology, 10:779-783). Other antibody production techniques are described in Antibodies: A Laboratory Manual, eds. Greenfield et al., Cold Spring Harbor Laboratory, 2012.

The antibody molecules of the invention can be prepared by synthetic techniques which are well known to the person skilled in the art, such as standard liquid peptide synthesis, or by solid-phase peptide synthesis methods. Alternatively, the antibody molecules may be prepared in solution using liquid phase peptide synthesis techniques, or further by a combination of solid-phase, liquid phase and solution chemistry.

The present invention further extends to the production of the antibody molecules of the invention by expression in a suitable expression system of a nucleic acid which encodes at least one amino acid sequence which, alone or in combination with one or more other amino acid sequences comprises an antibody molecule of the invention, such that a desired peptide or polypeptide can be encoded. For example, a nucleic acid encoding the amino acid light chain and a second nucleic acid encoding an amino acid heavy chain can be expressed to provide an antibody molecule of the present invention.

Traditional hybridoma techniques typically involve the immunisation of a mouse or other animal with an antigen in order to elicit production of lymphocytes capable of binding the antigen. The lymphocytes are isolated and fused with a myeloma cell line to form hybridoma cells which are then cultured in conditions which inhibit the growth of the parental myeloma cells but allow growth of the antibody producing cells. The hybridoma may be subject to genetic mutation, which may or may not alter the binding specificity of antibodies produced. Synthetic antibodies can be made using techniques known in the art (see, for example, Knappik et al, J. Mol. Biol. (2000) 296, 57-86 and Krebs et al, J. Immunol. Meth. (2001) 2154 67-84.

Modifications may be made in the VH, VL or CDRs of the binding members, or indeed in the FRs using any suitable technique known in the art. For example, variable VH and/or VL domains may be produced by introducing a CDR, e.g. CDR3 into a VH or VL domain lacking such a CDR. Marks et al. (1992) Bio/Technology, 10:779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences of the present invention may be produced.

Accordingly, in one embodiment of the invention, the present invention provides a method of generating a antibody molecule having specificity for RAMP2, the method comprising: (a) providing a starting repertoire of nucleic acids encoding a variable domain, wherein the variable domain includes a CDR1, CDR2 or CDR3 to be replaced or the nucleic acid lacks an encoding region for such a CDR; (b) combining the repertoire with a donor nucleic acid encoding a CDR amino acid sequence of an anti-RAMP2 antibody variable domain, the anti-RAMP2 antibody variable domain having the amino acid sequence shown as Seq ID No: 1, 2, 5, 6, 9, 10, 13, 14, 17, 18, 21, 22, 25, or 26 such that the donor nucleic acid is inserted into the CDR region in the repertoire so as to provide a product repertoire of nucleic acids encoding a variable domain; (c) expressing the nucleic acids of the product repertoire; (d) selecting a specific antigen-binding fragment specific for RAMP2; and (e) recovering the specific antigen-binding fragment or nucleic acid encoding it. The method may include an optional step of testing the antibody molecule for ability to inhibit adrenomedullin activity.

In another embodiment, the present invention provides a method of generating an antibody molecule having specificity for RAMP3, the method comprising: (a) providing a starting repertoire of nucleic acids encoding a variable domain, wherein the variable domain includes a CDR1, CDR2 or CDR3 to be replaced or the nucleic acid lacks an encoding region for such a CDR; (b) combining the repertoire with a donor nucleic acid encoding a CDR amino acid sequence of an anti-RAMP3 antibody variable domain, the anti-RAMP3 antibody variable domain having the amino acid sequence shown as Seq ID No: 29, 30, 33, 34, 37, 38, 41, 42, 45, 46, 49, 50, 53, or 54 such that the donor nucleic acid is inserted into the CDR region in the repertoire so as to provide a product repertoire of nucleic acids encoding a variable domain; (c) expressing the nucleic acids of the product repertoire; (d) selecting a specific antigen-binding fragment specific for RAMP3; and (e) recovering the specific antigen-binding fragment or nucleic acid encoding it. The method may include an optional step of testing the antibody molecule for ability to inhibit adrenomedullin activity.

Alternative techniques of producing variant antibody molecules of the invention may involve random mutagenesis of gene(s) encoding the VH or VL domain using, for example, error prone PCR (see Gram et al, 1992, P.N.A.S. 89 3576-3580). Additionally or alternatively, CDRs may be targeted for mutagenesis e.g. using the molecular evolution approaches described by Barbas et al 1991 PNAS 3809-3813 and Scier 1996 J Mol Biol 263 551-567.

Having produced such variants, antibodies and fragments, they may be tested for binding to RAMP2 or RAMP3 and for the ability to inhibit the activity of adrenomedullin.

As described herein, the inventors have demonstrated that antibody molecules according to the invention have an anti-angiogenic effect and/or anti-invasive effect. This therefore enables the use of antibody molecules of the invention as active therapeutic agents.

Accordingly in one embodiment of the invention, the antibody molecule is a “naked” antibody molecule. A “naked” antibody molecule is an antibody molecule which is not conjugated with an “active therapeutic agent”.

In the context of the present application, an “active therapeutic agent” is a molecule or atom which is conjugated to an antibody moiety (including antibody fragments, CDRs etc) to produce a conjugate. Examples of such “active therapeutic agents” include drugs, toxins, radiosensitisers radioisotopes, molecules which convert a prodrug into an active drug, cytotoxic chemotherapeutic agents, cytotoxic peptides, immunomodulators, chelators, boron compounds, dyes, nanoparticles etc.

In one embodiment of the invention, the antibody molecule is in the form of an immunoconjugate, comprising an antibody fragment conjugated to an “active therapeutic agent”.

Methods of producing immunoconjugates are well known in the art; for example, see U.S. Pat. No. 5,057,313, Shih et al., Int. J. Cancer 41:832-839 (1988); Shih et al., Int. J. Cancer 46:1101-1106 (1990), Wong, Chemistry Of Protein Conjugation And Cross-Linking (CRC Press 1991); Upeslacis et al., “Modification of Antibodies by Chemical Methods” in Monoclonal Antibodies: Principles And Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering And Clinical Application, Ritter et al. (eds.), pages 60-84 (Cambridge University Press 1995).

The antibody molecule of the invention may comprise further modifications. For example the antibody molecules can be glycosylated, pegylated, or linked to albumin or a nonproteinaceous polymer. The antibody molecule may be in the form of an immunoconjugate.

In one embodiment of the invention, the antibody molecules are non-fucosylated. ADCC activity of antibodies has been found to be dependent on the amount of fucose attached to the antibody. Non-fucosylated therapeutic antibodies have been shown to be more potent than corresponding fucosylated antibodies (Mori et al, Cytotechnology (2007) 55:109-114). Thus, in certain circumstances, for example where ESCSR is expressed in cancer endothelia of vessels feeding a tumour, a defucosylated antibody may be useful in enhancing ADCC targeting of such vessels. A number of techniques exist for the production of non-fucosylated antibodies by someone skilled in genetic engineering and molecular biology. These include, but are not limited to, removal of fucose post-production via incubation of the antibody with α-1,6 fucosidase; the genetic engineering of a mammalian cell to have reduced or no fucosylation by mutation of the fucosyltransferase genes, FUT8 or GMD; prevention of fucose being added by transfection with the glycosyl-N-transferase gene, GnTIII; or disruption of the FUT8 and/or GMD genes using siRNA. (see, for example, Jefferis R. Trends Pharmacol Sci. 2009 July; 30(7):356-62; Yamane-Ohnuki N, Satoh M. MAbs. 2009 May; 1 (3):230-6; Mori K, et al. Cytotechnology. 2007 December; 55(2-3):109-14.

Antibody molecules of the invention may be labelled. Labels which may be used include radiolabels, enzyme labels such as horseradish peroxidase, alkaline phosphatase, or biotin. As described in the Examples, antibodies of the invention have been shown to bind to cancer cells. Accordingly, labelled antibody molecules may be useful for imaging tumours, in detection of tumours, and in diagnostic or prognostic imaging methods.

Indeed in another aspect of the invention, there is provided a method of imaging tumour neovasculature in an individual, the method comprising:
administering to the individual an antibody molecule of the invention, in which the antibody molecule comprises a label moiety, and
detecting the location of the label moiety in the individual.

A further aspect of the invention comprises a method of detecting tumour cells, e.g. a solid tumour, in cells, tissue or organ of an individual, the method comprising:
administering an antibody molecule of the invention, in which the antibody molecule comprises a label moiety to said cells, tissue, organ or individual, and
detecting the presence and/or location of the label moiety in the cells, tissue, organ or individual.

The method may be performed in vivo, ex vivo or in vitro as appropriate.

The method may be used to diagnose the presence of a tumour or may be used to monitor progression of a tumour and/or response to particular treatment regimens, for example, by repeating the method at time intervals during a patient's treatment and determining differences in the size, vascularisation, or other characteristic(s), of tumour detected.

The ability of an antibody molecule to inhibit adrenomedullin activity may be tested using any suitable method. For example the ability of an antibody molecule to inhibit angiogenesis may be tested using any suitable assay known in the art. For example, such ability may be tested using the assays as described in the Examples, for example a Matrigel based assay, using, for example HUVEC cells seeded in Matrigel in the presence of the antibody molecule and appropriate controls. The formation of tube-like vessels may be monitored and quantified by measuring the reduction in, for example, branching points, the number of vessels, mean vessel length and maximum vessel length compared to controls.

The ability of an antibody molecule to inhibit tumour cell invasion may be tested using any suitable invasion assay known in the art. For example, such ability may be tested using a modified Boyden chamber. An antibody molecule is considered to inhibit tumour cell invasion if it has the ability to inhibit invasion by a statistically significant amount. For example, in one embodiment, the antibody molecule is able to inhibit invasion by at least 10%, for example at least 25%, at least 50%, at least 60%, at least 70%, at least 80% or at least 90% when compared to an appropriate control antibody.

The ability of an antibody molecule to inhibit the chemotactic activity of adrenomedullin may be tested using a HUVEC migration assay. In such an assay, an antibody molecule is considered to inhibit the chemotactic activity of adrenomedullin if it has the ability to inhibit the migration of HUVEC cells towards an endothelial cell supplement by a statistically significant amount. For example, in one embodiment, the antibody molecule is able to inhibit the chemotactic activity by at least 10%, for example at least 15%, at least 20%, at least 30%, at least, at least 40%, at least 50%, at least 60%, at least 70%, at least 80% or at least 90% when compared to an appropriate control antibody. An appropriate control antibody may be a control antibody of the same isotype which does not inhibit the chemotactic effect of adrenomedullin for example an IgG1 isotype control antibody.

An alternative assay which may be used is a wound assay. The extent of “wound” closure may be blindly assessed microscopically by an independent investigator and quantified, for example using a calibrated eyepiece graticule. The extent of closure in the antibody treated slides may be compared to time matched treated controls and the % inhibition of wound closure compared to time matched controls calculated.

In certain embodiments of the invention, the antibody molecule of the invention has the ability to induce or accelerate necrosis in the tumour vasculature or elsewhere in a tumour. The ability of an antibody molecule to induce or accelerate necrosis may be assessed using any suitable means. For example, such ability may be assessed by histological examination of tumour tissue obtained from a xenograft model of the cancer under investigation. For example, the assessment may be carried out using, for example, a xenograft model using glioblastoma U87MG tumours, as described in the examples, with staining for caspase 3 used to identify necrotic cells.

In one embodiment, an antibody molecule is considered to induce or accelerate apoptosis if compared to the % area of necrotic cells in the tumour histological section obtained from the animal treated with a control, for example PBS or a control antibody, the % area of necrotic cells in the tumour histological section obtained from the animal treated with the antibody molecule is more than 2 times greater, such as more than 4 times greater, such as more than 5 times greater, for example more than 7 times greater, more than 10 times greater, more than 12 times greater or more than 15 times greater.

### Nucleic Acid

Nucleic acid of and for use in the present invention may comprise DNA or RNA. It may be produced recombinantly, synthetically, or by any means available to those in the art, including cloning using standard techniques.

As described above, the invention encompasses the nucleic acid which encodes the VL or VH chains of antibody molecules of the invention. Accordingly, in one embodiment, nucleic acids of the invention may comprise:
(i) the nucleotide sequence shown as VL 1H6 (Sequence ID No: 3), and/or the nucleotide sequence shown as VH 1H6 (Sequence ID No: 4);
(ii) the nucleotide sequence shown as VL 1A7 (Sequence ID No: 7), and/or the nucleotide sequence shown as VH 1A7 (Sequence ID No: 8); or
(iii) the nucleotide sequence shown as VL 2A6 (Sequence ID No: 11), and/or the nucleotide sequence shown as VH 2A6 (Sequence ID No: 12); or
(iv) the nucleotide sequence shown as VL 2A8 (Sequence ID No: 15), and/or the nucleotide sequence shown as VH 2A8 (Sequence ID No: 16); or
(v) the nucleotide sequence shown as VL 2H3 (Sequence ID No: 19), and/or the nucleotide sequence shown as VH 2H3 (Sequence ID No: 20); or
(vi) the nucleotide sequence shown as VL 1H7 (Sequence ID No: 23), and/or the nucleotide sequence shown as VH 1H7 (Sequence ID No: 24); or
(vii) the nucleotide sequence shown as VL 1A10 (Sequence ID No: 27), and/or the nucleotide sequence shown as VH 1A10 (Sequence ID No: 28); or
(viii) the nucleotide sequence shown as VL 3A5 (Sequence ID No: 31), and/or the nucleotide sequence shown as VH 3A5 (Sequence ID No: 32); or
(ix) the nucleotide sequence shown as VL 3A12 (Sequence ID No: 35), and/or the nucleotide sequence shown as VH 3A12 (Sequence ID No: 36); or
(x) the nucleotide sequence shown as VL 4C10 (Sequence ID No: 39), and/or the nucleotide sequence shown as VH 4C10 (Sequence ID No: 40); or
(xi) the nucleotide sequence shown as VL 5H9 (Sequence ID No: 43), and/or the nucleotide sequence shown as VH 5H9 (Sequence ID No: 44); or
(xii) the nucleotide sequence shown as VL 6B8 (Sequence ID No: 47), and/or the nucleotide sequence shown as VH 6B8 (Sequence ID No: 48); or
(xiii) the nucleotide sequence shown as VL 6C2 (Sequence ID No: 51), and/or the nucleotide sequence shown as VH 6C2 (Sequence ID No: 52; or
(xiv) the nucleotide sequence shown as VL 6D8 (Sequence ID No: 55), and/or the nucleotide sequence shown as VH 6D8 (Sequence ID No: 56).

The nucleic acid may be inserted into any appropriate vector. A vector comprising a nucleic acid of the invention forms a further aspect of the present invention. In one embodiment the vector is an expression vector and the nucleic acid is operably linked to a control sequence which is capable of providing expression of the nucleic acid in a host cell. A variety of vectors may be used. For example, suitable vectors may include viruses (e. g. vaccinia virus, adenovirus, etc.), baculovirus; yeast vectors, phage, chromosomes, artificial chromosomes, plasmids, or cosmid DNA.

The vectors may be used to introduce the nucleic acids of the invention into a host cell. A wide variety of host cells may be used for expression of the nucleic acid of the invention. Suitable host cells for use in the invention may be prokaryotic or eukaryotic. They include bacteria, e.g. E. coli, yeast, insect cells and mammalian cells. Mammalian cell lines which may be used include Chinese hamster ovary cells, baby hamster kidney cells, NSO mouse melanoma cells, monkey and human cell lines and derivatives thereof and many others.

A host cell strain that modulates the expression of, modifies, and/or specifically processes the gene product may be used. Such processing may involve glycosylation, ubiquitination, disulfide bond formation and general post-translational modification.

Accordingly, the present invention also provides a host cell, which comprises one or more nucleic acid or vectors of the invention.

Also encompassed by the invention is a method of production of an antibody molecule of the invention, the method comprising culturing a host cell comprising a nucleic acid of the invention under conditions in which expression of the nucleic antibody molecule from the nucleic acid occurs and, optionally, isolating and/or purifying the antibody molecule.

For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 5th ed., Ausubel et al. eds., John Wiley & Sons, 2005 and, Molecular Cloning: a Laboratory Manual: 3rd edition Sambrook et al., Cold Spring Harbor Laboratory Press, 2001.

### Treatment

Treatment” includes any regime that can benefit a human or non-human animal. The treatment may be in respect of an existing condition or may be prophylactic (preventative treatment). Treatment may include curative, alleviation or prophylactic effects.

The antibody molecule and nucleic acids of the invention may be used in the treatment of a variety of conditions and disorders. These include neoplastic disease/cancer and other diseases associated with angiogenesis.

“Treatment of cancer” includes treatment of conditions caused by cancerous growth and/or vascularisation and includes the treatment of neoplastic growths or malignant tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, prostate, cervical and ovarian carcinoma, non-small cell lung cancer, hepatocellular carcinoma, lymphomas, including Hodgkin and non-Hodgkin lymphomas, neuroblastoma, melanoma, myeloma, Wilms tumor, astrocytomas, gliomas and retinoblastomas.

In one particular embodiment, the antibody molecules or nucleic acids of the invention may be used in the treatment of glioblastoma.

In another embodiment of the invention, the antibody molecules or nucleic acids of the invention may be used in the treatment of renal cancer.

The invention may be particularly useful in the treatment of existing cancer and in the prevention of the recurrence of cancer after initial treatment or surgery.

The antibody molecules, nucleic acids and compositions of the invention may also be used in the treatment of other disorders mediated by or associated with angiogenesis. Such conditions include, for example, benign tumours, various autoimmune disorders, hereditary disorders, ocular disorders.

The methods of the present invention may be used to treat angiogenesis-mediated disorders including hemangioma, solid tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, peptic ulcer, Helicobacter related diseases, fractures, keloids, and vasculogenesis.

The antibody molecules, nucleic acids and methods of the invention may also be used to treat angiogenesis associated inflammation, including various forms of arthritis, such as rheumatoid arthritis and osteoarthritis.

Further, in these methods, treatment with combinations of the compounds described herein with other agents useful for treating the disorders is provided. Such agents include, for instance, cyclooxygenase-2 (COX-2) inhibitors, which are well known to those of skill in the art.

The blood vessels in the synovial lining of the joints can undergo angiogenesis. The endothelial cells form new vascular networks and release factors and reactive oxygen species that lead to pannus growth and cartilage destruction. These factors are believed to actively contribute to rheumatoid arthritis and also to osteoarthritis. Chondrocyte activation by angiogenic-related factors contributes to joint destruction, and also promotes new bone formation. The antibody molecules, nucleic acids and methods described herein may be used as a therapeutic intervention to prevent bone destruction and new bone formation.

Pathological angiogenesis is also believed to be involved with chronic inflammation. Examples of disorders that can be treated using the methods described herein also include ulcerative colitis, Crohn's disease, and atherosclerosis.

Adrenomedullin has vasodilatory activity and thus antibody molecules of the invention may be used in the treatment of conditions characterized by excessive vasodilation or conditions or symptoms in which vasoconstriction may be beneficial.

It is known that in the later phase of sepsis, adrenomedullin constitutes a risk factor that is strongly associated with the mortality of patients in septic shock. In an embodiment antibody molecules of the invention may thus be used for the treatment of sepsis in the late stage of sepsis and in septic shock.

Other conditions for which antibodies against adrenomedullin have been described (see, for example WO 2004/097423A1, WO 2006 027147A1, and PCT/EP2005/012844) and in which, in one embodiment, antibody molecules of the invention may be used include, for example, cardiovascular diseases, infections, dermatological diseases, neurological diseases, urological disease, endocrinological diseases, metabolic diseases, gastroenterological diseases, hematological diseases and respiratory diseases.

### Pharmaceutical Compositions

The antibody molecules and nucleic acids may be administered as a pharmaceutical composition. Pharmaceutical compositions according to the present invention, and for use in accordance with the present invention, may comprise, in addition to active ingredients, a pharmaceutically acceptable excipient, a carrier, buffer stabiliser or other materials well known to those skilled in the art (see, for example, see Remington: the Science and Practice of Pharmacy, 23^{rd} edition, Adejare A, et al, eds., Academic Press, 2020. Such materials may include buffers such as acetate, Tris, phosphate, citrate, and other organic acids; antioxidants; preservatives; proteins, such as serum albumin, gelatin, or immunoglobulins; hydrophilic polymers such aspolyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compounds selected as necessary for the particular indication being treated, preferably with complementary activities that do not adversely affect the activity of the binding member, nucleic acid or composition of the invention. For example, in the treatment of cancer, in addition to an anti-RAMP2 or anti-RAMP3 antibody molecule of the invention, the formulation may comprise an additional antibody which binds a different epitope on anti-RAMP2 or anti-RAMP3 or an antibody to some other target such as adrenomedullin or e.g. a growth factor that e.g. affects the growth of the particular cancer, and/or a chemotherapeutic agent.

The active ingredients (e.g. antibody molecule and/or chemotherapeutic agents) may be administered via microspheres, microcapsules liposomes, other microparticulate delivery systems. For example, active ingredients may be entrapped within microcapsules which may be prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 23^{rd} edition, Adejare A, et al, eds., Academic Press, 2020.

Sustained-release preparations may be used for delivery of active agents. Suitable examples of sustained-release preparations include semi-permeable matrices of solid hydrophobic polymers containing the antibody, which matrices are in the form of shaped articles, e. g. films, suppositories or microcapsules. Examples of sustained-release matrices include polyesters, hydrogels (for example, poly(2-hydroxyethyl-methacrylate), or poly (vinylalcohol)), polylactides (U.S. Pat. No. 3,773,919), copolymers of L-glutamic acid and ethyl L-glutamate, non-degradable ethylene-vinyl acetate, degradable lactic acid-glycolic acid copolymers, and poly-D-(−)-3-hydroxybutyric acid.

As described above nucleic acids of the invention may also be used in methods of treatment. Nucleic acid of the invention may be delivered to cells of interest using any suitable technique known in the art. Nucleic acid (optionally contained in a vector) may be delivered to a patient's cells using in vivo or ex vivo techniques. For in vivo techniques, transfection with viral vectors (such as adenovirus, Herpes simplex I virus, or adeno-associated virus) and lipid-based systems (useful lipids for lipid-mediated transfer of the gene are DOTMA, DOPE and DC-Chol, for example) may be used (see for example, Anderson et al., Science 256:808-813 (1992). See also WO 93/25673).

In ex vivo techniques, the nucleic acid is introduced into isolated cells of the patient with the modified cells being administered to the patient either directly or, for example, encapsulated within porous membranes which are implanted into the patient (see, e.g. U.S. Pat. Nos. 4,892,538 and 5,283,187). Techniques available for introducing nucleic acids into viable cells may include the use of retroviral vectors, liposomes, electroporation, microinjection, cell fusion, DEAE-dextran, the calcium phosphate precipitation method, etc.

The binding member, agent, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

### Dose

The antibody molecules, nucleic acids or compositions of the invention are preferably administered to an individual in a “therapeutically effective amount”, this being sufficient to show benefit to the individual. The optimal dose and actual dosage regimen can be determined by physicians and may depend on a number of factors including the condition being treated, its severity, the age, sex, and weight of the patient being treated, the agent being used, and the route of administration, and will be at the discretion of the physician.

As a rough guideline, doses of antibodies may be given in amounts of 1 ng/kg-500 mg/kg of patient weight.

The invention will now be described further in the following non-limiting examples. Reference is made to the accompanying figures in which:

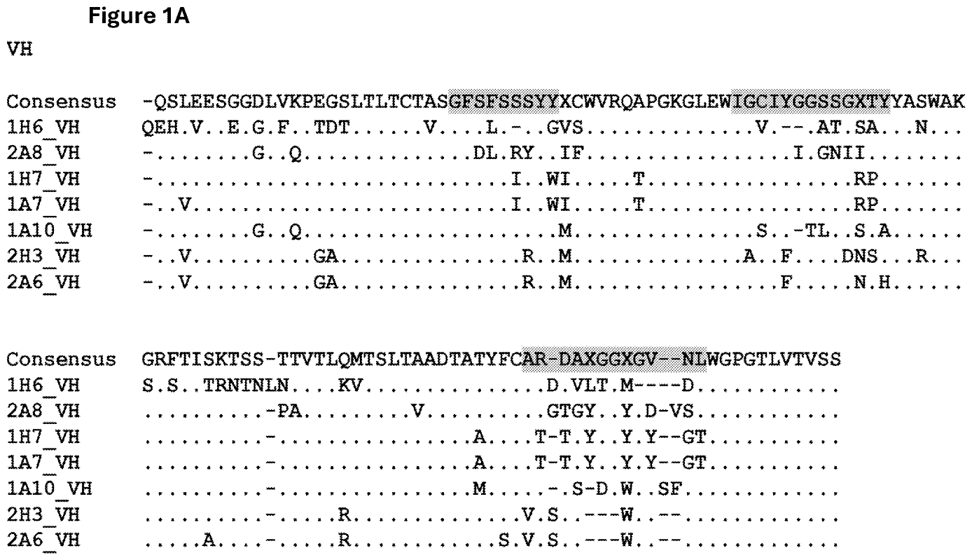

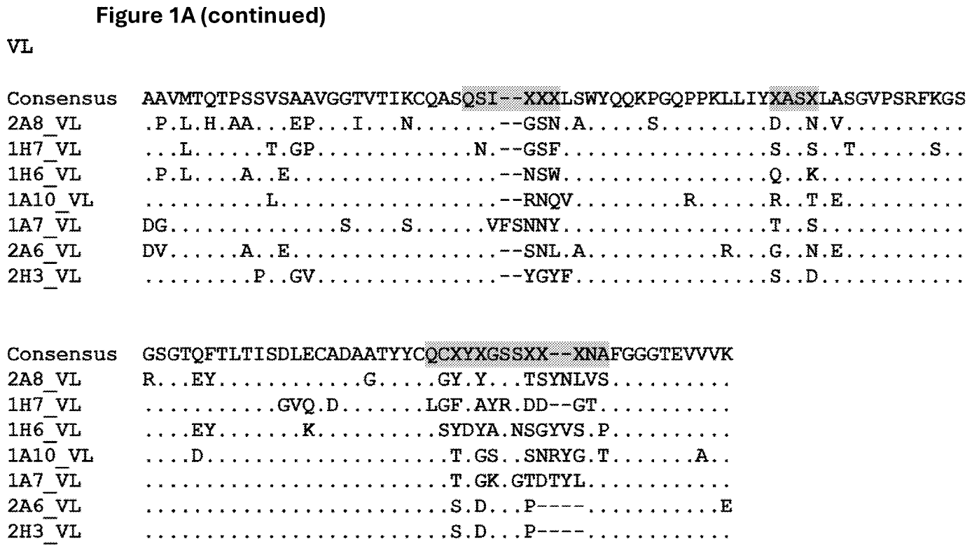

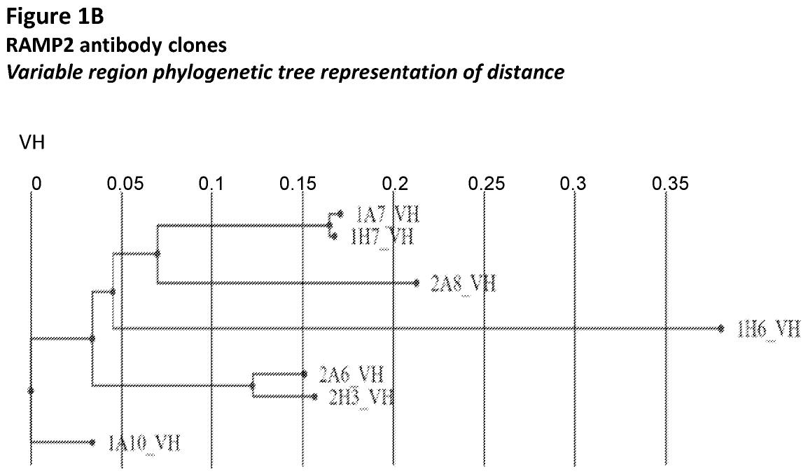

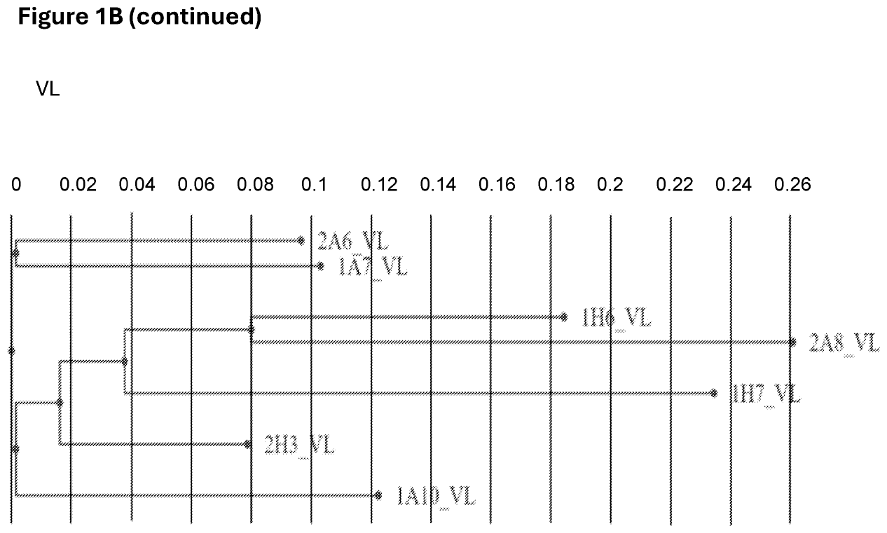

FIG. 1A shows variable region sequence alignment of RAMP2 antibody clones;

FIG. 1B shows a phylogenetic tree of the variable regions of the RAMP2 antibody clones;

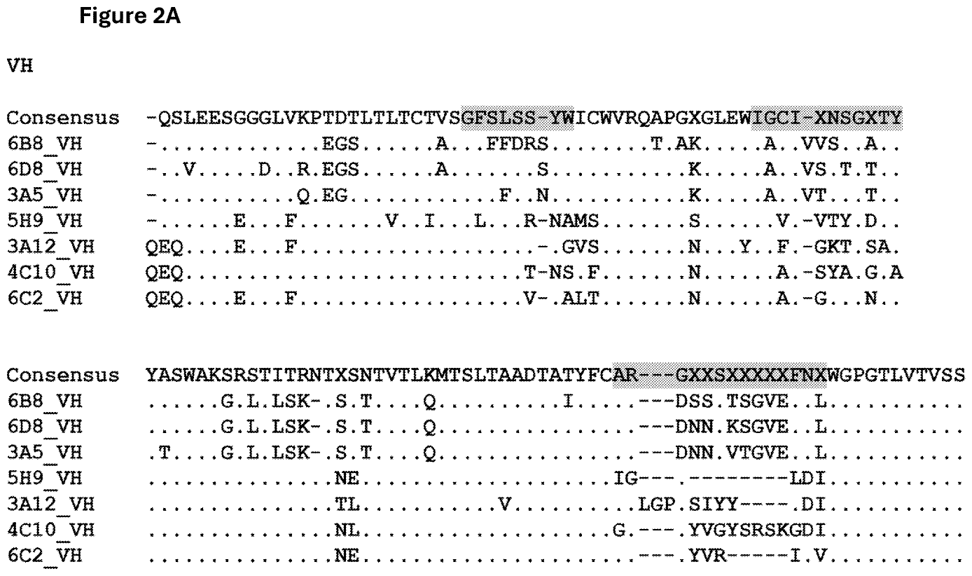

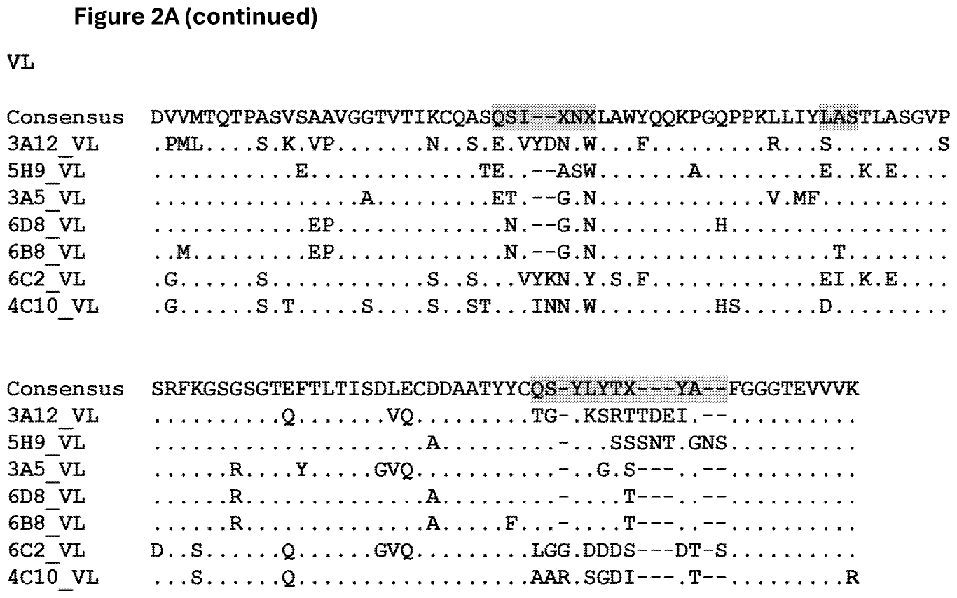

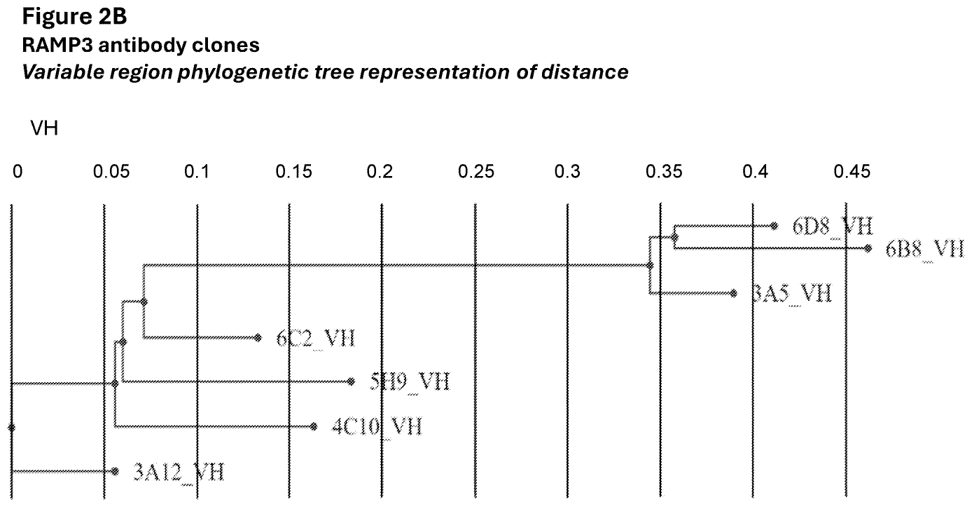

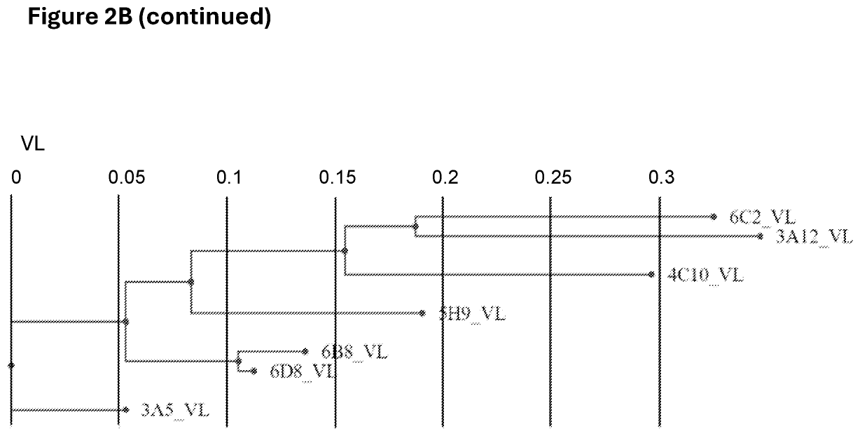

FIG. 2A shows variable region sequence alignment of RAMP3 antibody clones;

FIG. 2B shows a phylogenetic tree of the variable regions of the RAMP3 antibody clones;

FIG. 3 shows a comparison of CDRs of anti-RAMP2 antibodies isolated from rabbit. Heavy chain CDR-s are designated H1, H2, H3 and light chain CDR-s L1, L2, L3, respectively. Distances between individual unique CDR regions are shown in distance matrix.

FIG. 4 shows a comparison of CDR-s of anti-RAMP3 antibodies isolated from rabbit. Heavy chain CDR-s are designated H1, H2, H3 and light chain CDR-s L1, L2, L3, respectively. Distances between individual unique CDR regions are shown in distance matrix.

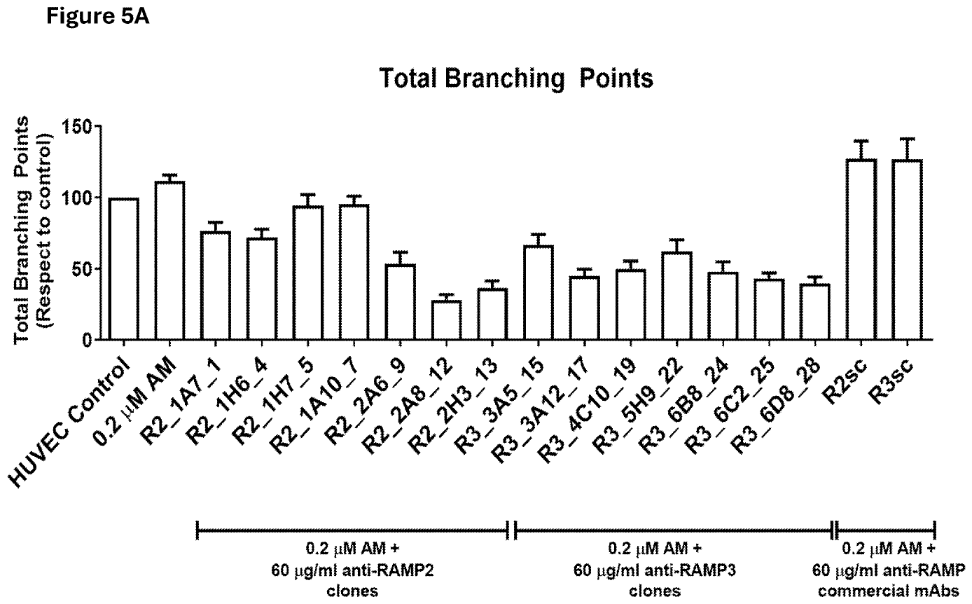

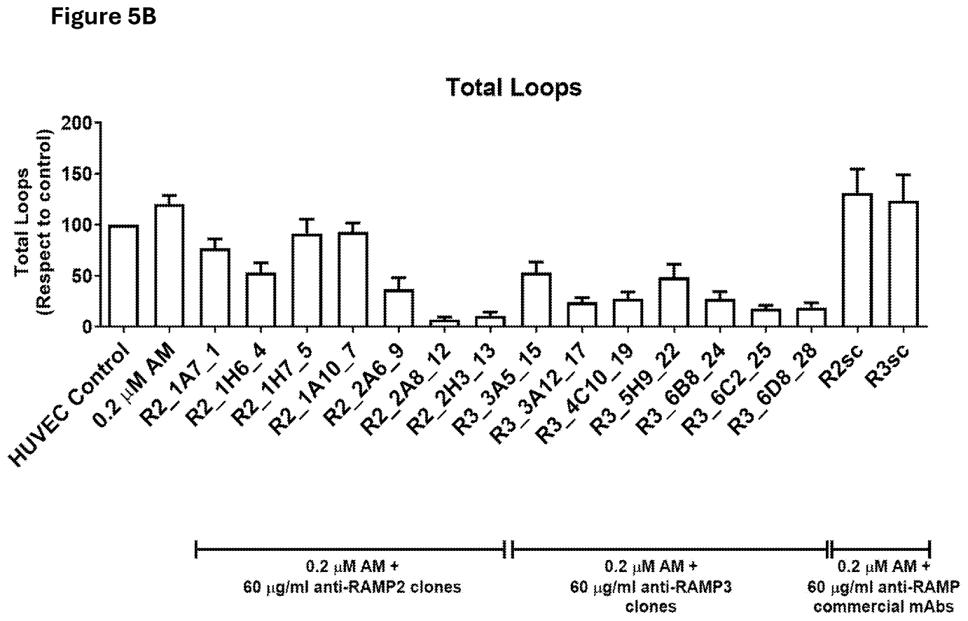

FIG. 5 shows the anti-angiogenic effect on endothelial HUVEC cell angiogenesis by 14 clones of RAMP2 and RAMP3 Abs and two available anti-RAMP2 and anti-RAMP3 commercial antibodies developed by Santa Cruz Biotechnology, Inc. Endothelial cells were pretreated for 30 min with 60 μg/ml each of anti-RAMP2 antibodies or anti-RAMP3. For this tube-formation assay, medium containing 0.5% FBS was supplemented with 2.10⁻⁷ cells and plates were incubated for 5 h at 37°. For the quantitative image analysis, the inventors evaluated the Total Branching Points (A) and Total Loops (B) using the WimTube analysis. Cut-off for the screening 50% of inhibition for each parameters. Data are expressed as mean±SEM of 3 independent experiments.

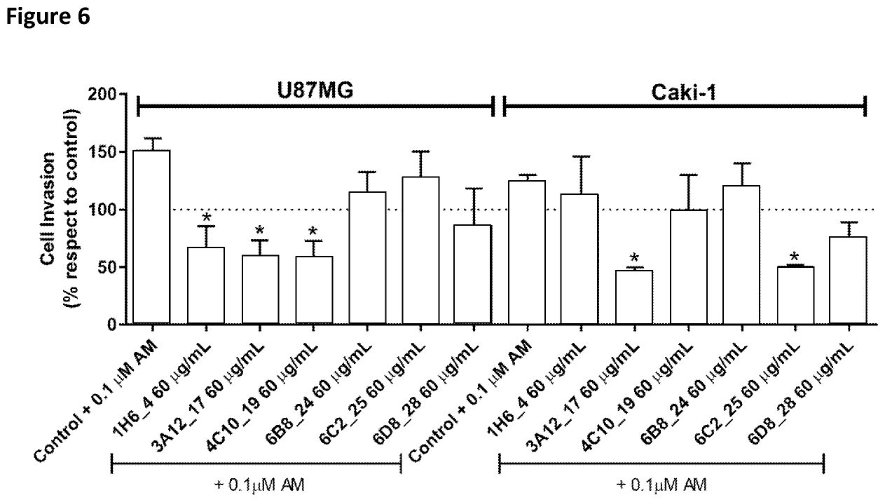

FIG. 6 shows the effect of 0.1 μM AM and RAMPs clones on U87MG and Caki-1 cells migration and invasion. Cells were pretreated for 120 min with 60 μg/ml each of anti-RAMP2 or anti-RAMP3 antibodies (AMR Abs) and then placed in the upper compartment of a 24-well Boyden chamber. 0.1 μM AM was added in lower wells. Cells migrating through the filter in absence or presence of Matrigel were counted after 24 h. Data are expressed as the number of migrated cells in 10 high-power fields, and the values represent the mean±SEM of 3 independent experiments each performed in duplicate (*p<0.05).

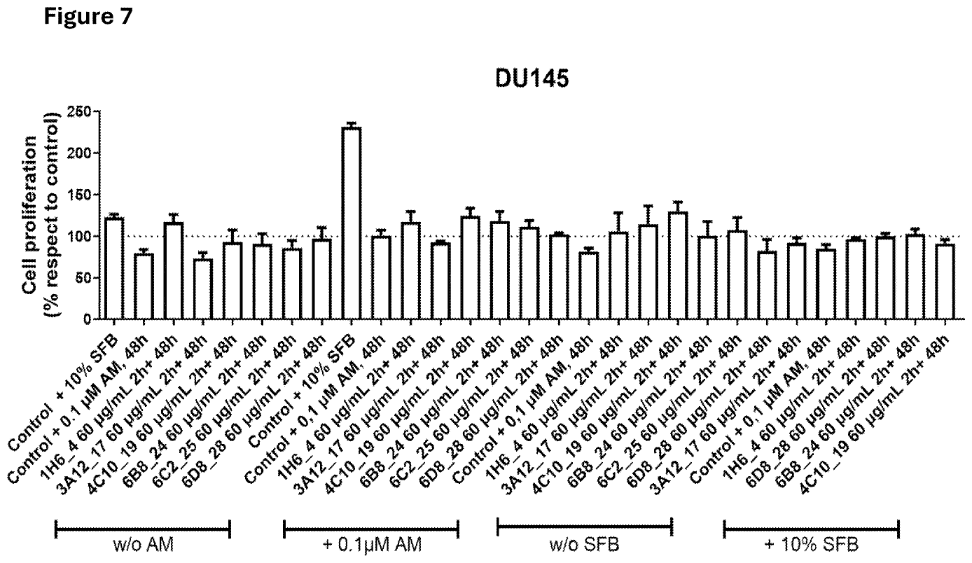

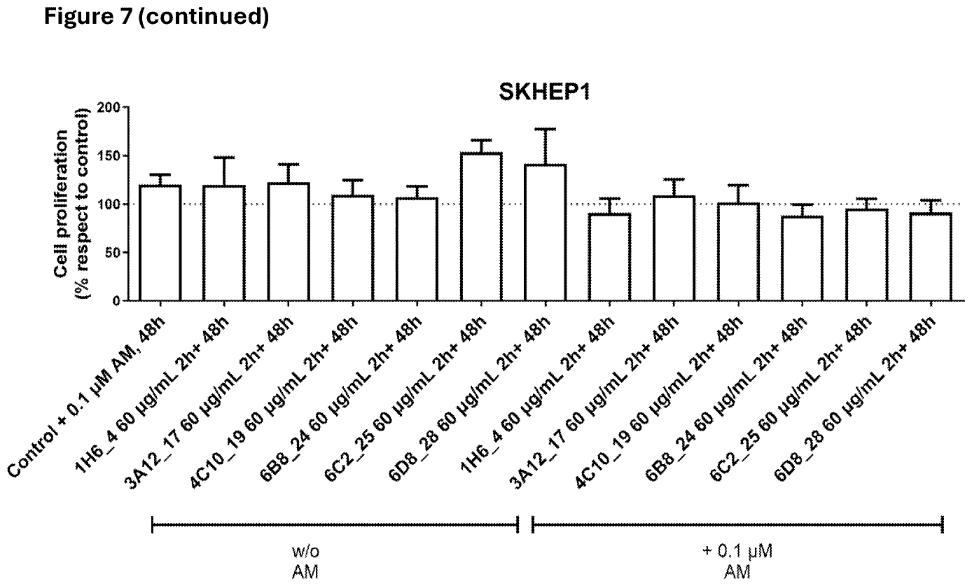

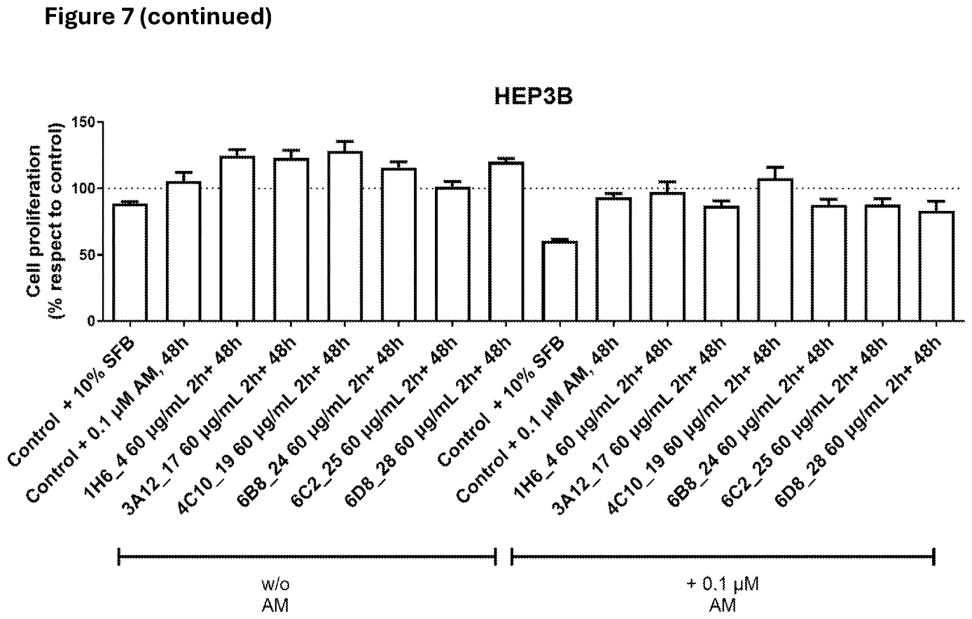

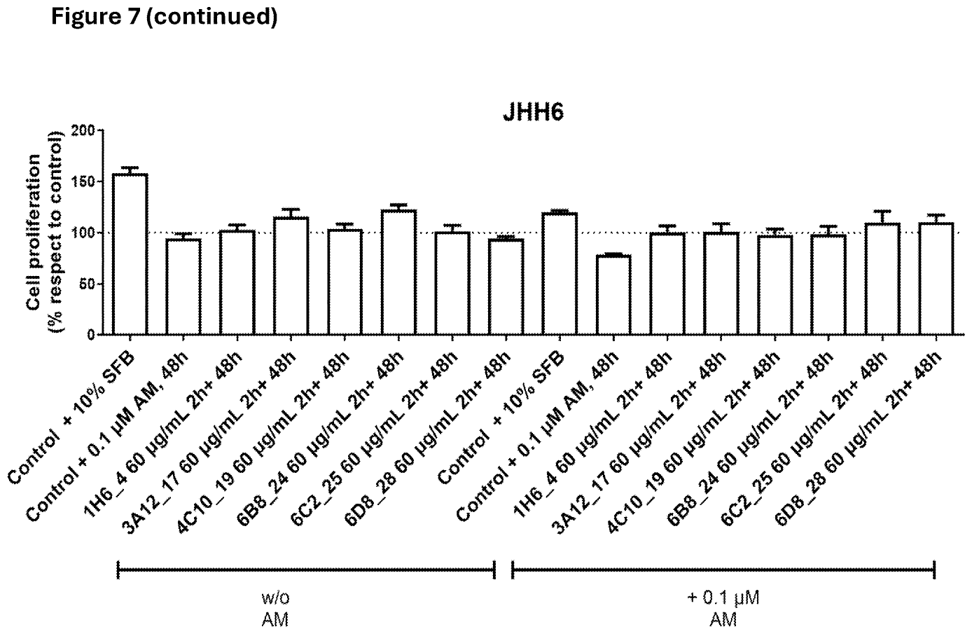

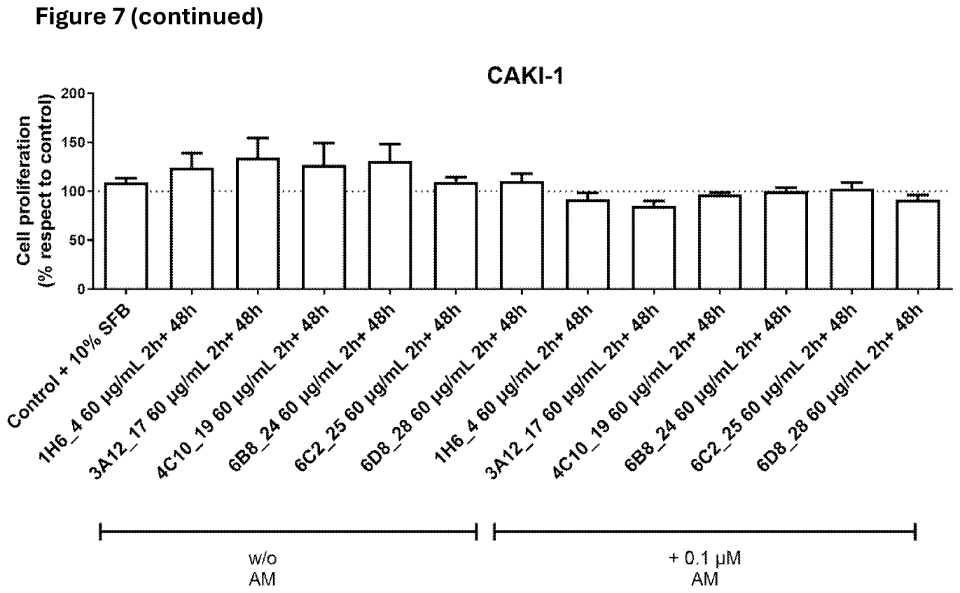

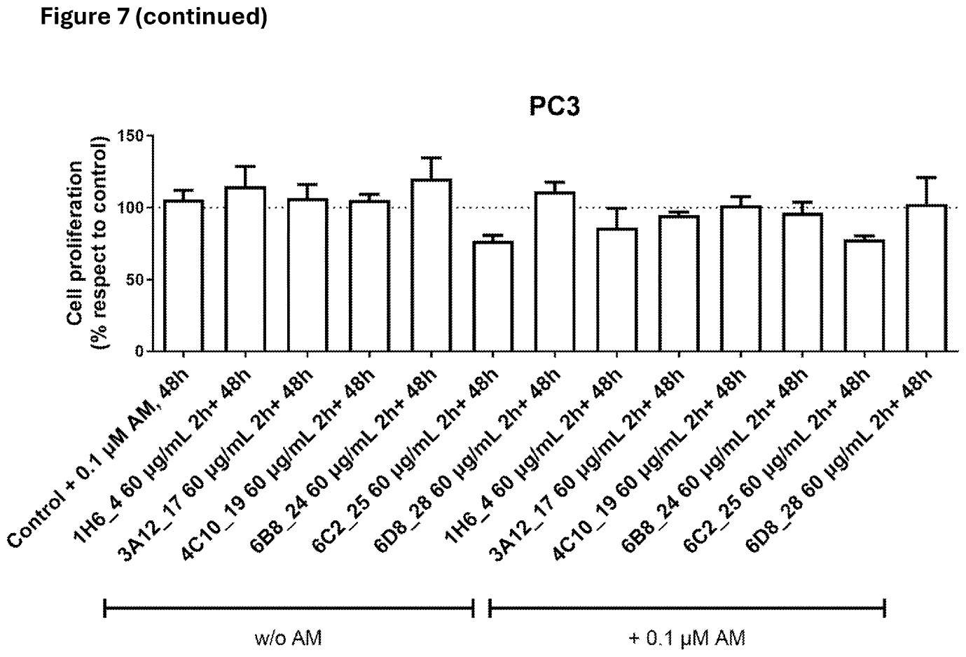

FIG. 7 shows the effect of of AM and RAMP antibodies clones on the proliferation of DU145, SKHEP1, HEP3B, JHH6, Caki-1 and PC3 cell lines. Cells were seeded at the density of 3.10⁴ per well in 96 multiwell plates. After 12 h, cells were preincubated with 60 μg/ml of RAMP antibodies clones for 120 min. After, culture medium was added supplemented with 10% FBS, or of 0.1 μM AM. Cell proliferation was measured after 48 h-incubation using MTT method.

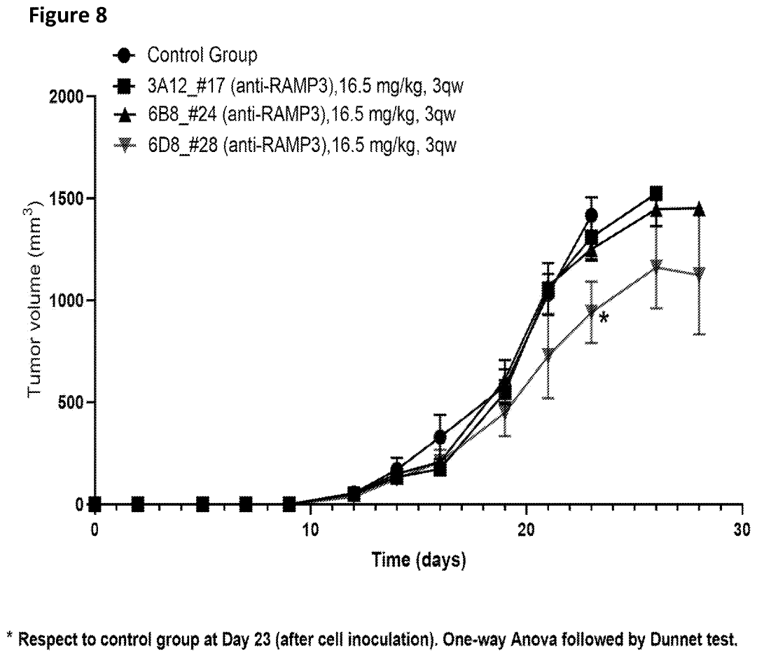

FIG. 8 shows tumor volume curves of U87MG-bearing mice in control and treated groups. U87MG cell line-derived murine xenografts were treated with 16.5 mg/kg of 3A12 #17, 6B8_#24 and 6D8_#28 RAMPs Abs or vehicle (PBS) per ip, 3 days a week. Tumor weight was indirectly monitored every 2-3 days throughout the experiment by measuring their dimensions with a caliper. * p<0.05 respect to control group at Day 23, one-way Anova followed by Dunnet test.

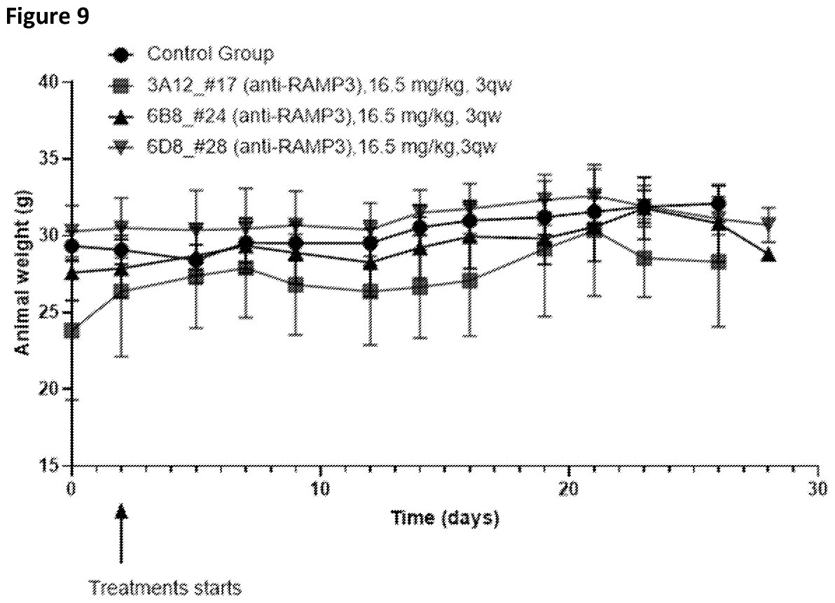

FIG. 9 shows animal weight in control and RAMPs Abs-treated U87MG-bearing mice. Body weight was evaluated from U87MG cell injection throughout the treatment period.

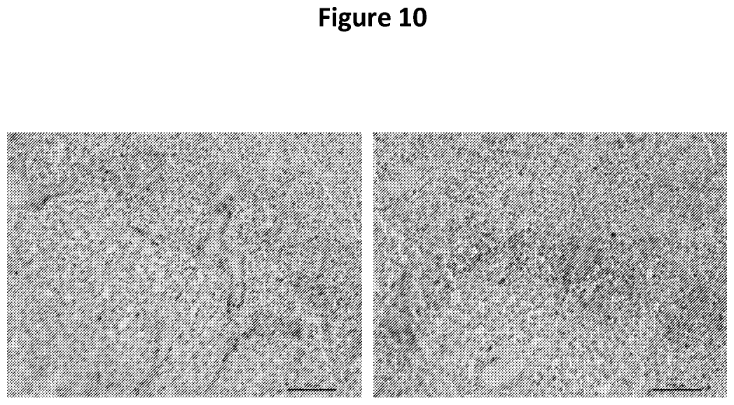

FIG. 10 shows a pair of representative microscopic images of a U87MG tumour tissue section stained for necrosis (brown=necrotic cells)—PBS control group.

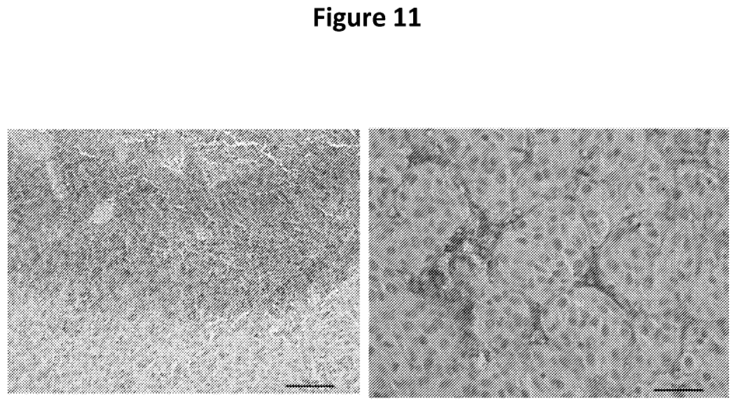

FIG. 11 shows representative microscopic images of U87MG tumour tissue sections stained for necrosis (brown=necrotic cell)—RAMP3 6D8 #28 treated group. Left-large defined area of progressively increasing necrosis. Right-close up of vascular associated cells in otherwise healthy tissue showing necrotic epithelial cells

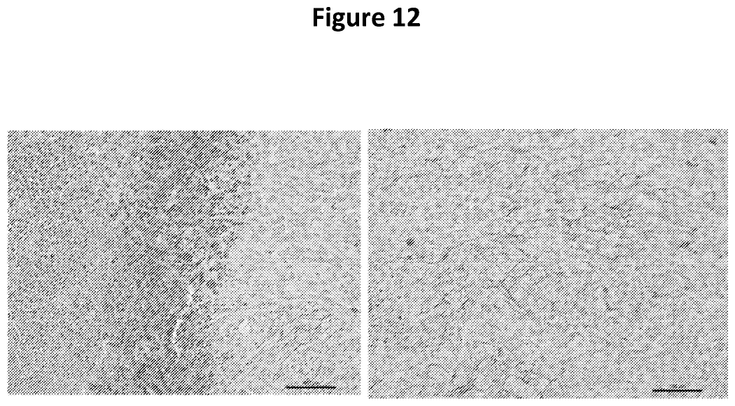

FIG. 12 shows representative microscopic images of U87MG tumour tissue sections stained for necrosis (brown=necrotic cell)—RAMP3 6B8 #24 treated group. Left-large defined area of progressively increasing necrosis. Right-close up of vascular associated cells in otherwise healthy tissue showing necrotic epithelial cells

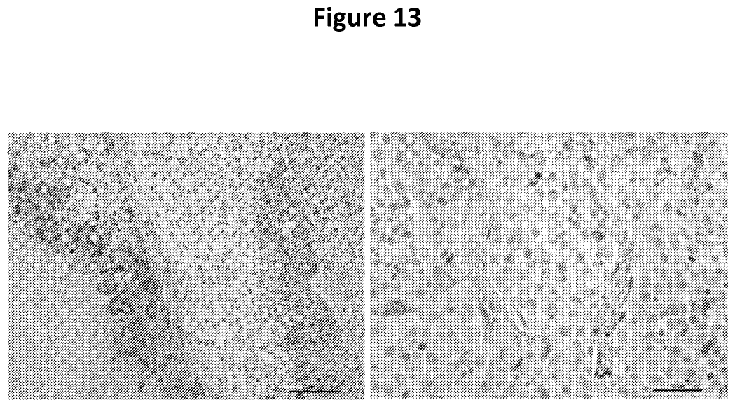

FIG. 13 shows representative microscopic images of U87MG tumour tissue sections stained for necrosis (brown=necrotic cell)—RAMP3 3A12 #17 treated group. Left-large area of progressively increasing necrosis, less firmly bordered than other treatment groups. Right-close up of vascular associated cells in otherwise healthy tissue showing necrotic epithelial cells

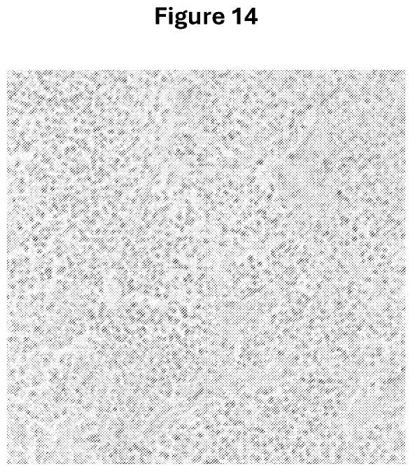

FIG. 14 shows a representative microscopic image of a U87MG tumour tissue section stained for caspase 3 (brown=apoptotic cells)—PBS control group

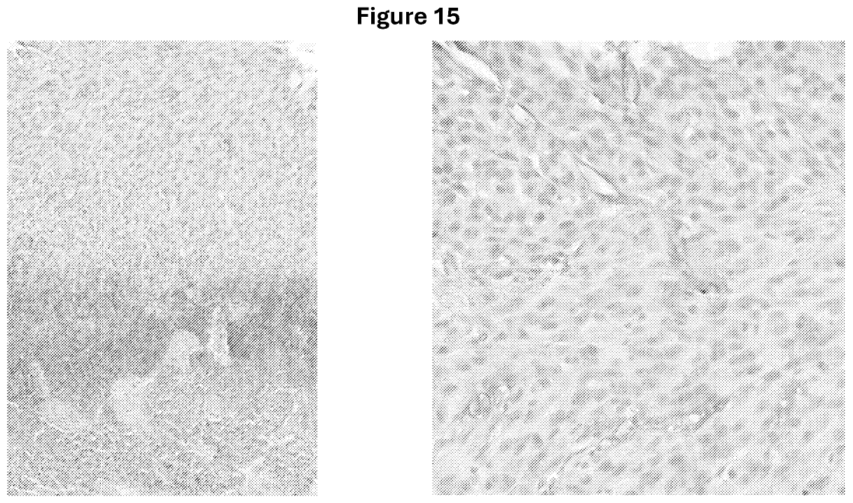

FIG. 15 shows representative microscopic images of U87MG tumour tissue sections stained for caspase-3 (brown=apoptotic cell)—RAMP3 6D8 #28 treated group. Left-large defined area of progressively increasing apoptosis and cell death. Right-close up of vascular associated cells in otherwise healthy tissue showing apoptotic epithelial cells

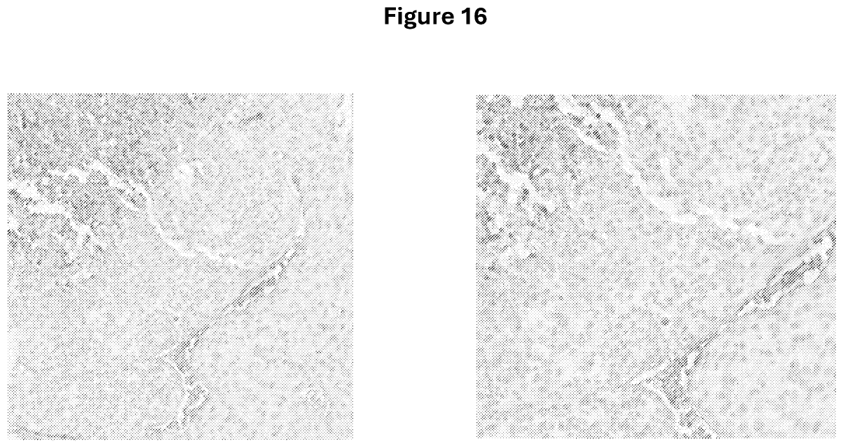

FIG. 16 shows representative microscopic images of U87MG tumour tissue sections stained for caspase-3 (brown=apoptotic cell)—RAMP3 6B8 #24 treated group. Left-large defined area of progressively increasing apotosis and cell death. Right-close up of vascular associated cells in otherwise healthy tissue showing apoptotic epithelial cells

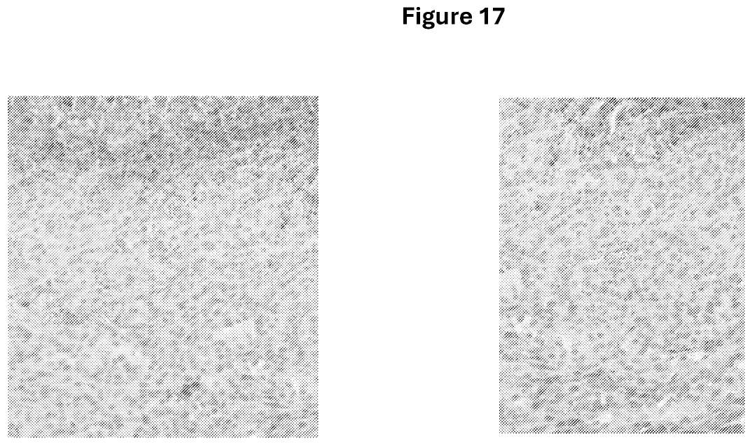

FIG. 17 shows Representative microscopic images of U87MG tumour tissue sections stained for caspase-3 (brown=apoptotic cell)—RAMP3 3A12 #17 treated group. Left-large area of progressively increasing apototic cells, less firmly bordered than other treatment groups. Right-close up of vascular associated cells in otherwise healthy tissue showing apoptotic epithelial cells

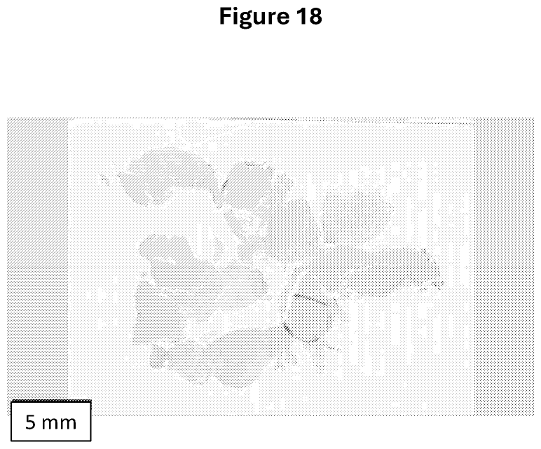

FIG. 18 shows an example of stained tissue section taken from U87MG xenograft in mouse treated with PBS vehicle control only (Mouse CTRL-1 in table above). Blue—healthy tissue, Yellow—positive staining for Caspase-3 indicating active apoptosis (note that image has been presented with colour saturation at 300% to enhance visibility of contrast in compressed format)

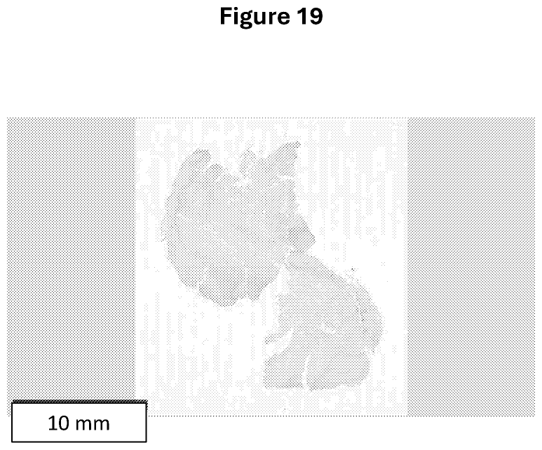

FIG. 19 shows an example of a stained tissue section taken from U87MG xenograft in mouse treated with Ab6D8 #28 (Mouse Ab6D8 #28-1 in table above). Blue—healthy tissue, Yellow—positive staining for Caspase-3 indicating active apoptosis (note that image has been presented with colour saturation at 300% to enhance visibility of contrast in compressed format)

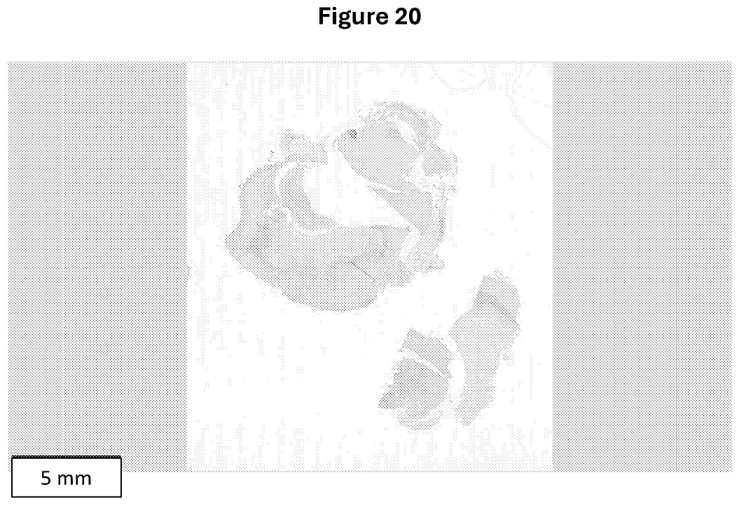

FIG. 20 shows an example of a stained tissue section taken from U87MG xenograft in mouse treated with Ab6B8 #24 (Mouse Ab6B8 #24-2 in table above). Blue—healthy tissue, Yellow—positive staining for Caspase-3 indicating active apoptosis (note that image has been presented with colour saturation at 300% to enhance visibility of contrast in compressed format)

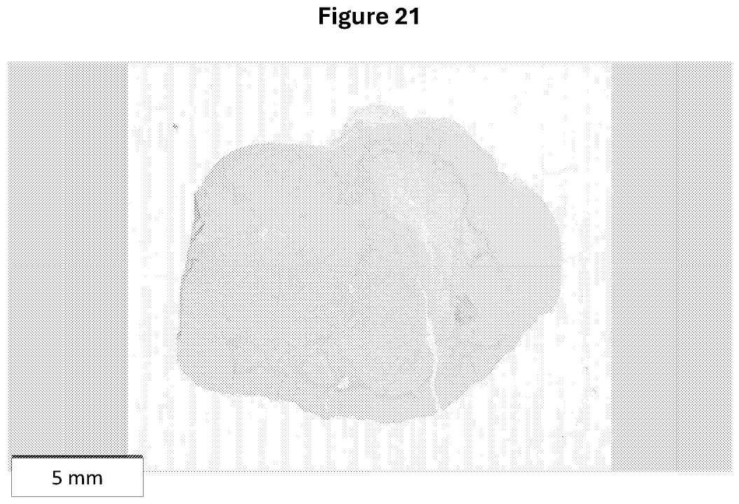

FIG. 21 shows an example of a stained tissue section taken from U87MG xenograft in mouse treated with Ab3A12 #17 (Mouse Ab3A12 #17-1 in table above). Blue—healthy tissue, Yellow—positive staining for Caspase-3 indicating active apoptosis (note that image has been presented with colour saturation at 300% to enhance visibility of contrast in compressed format)

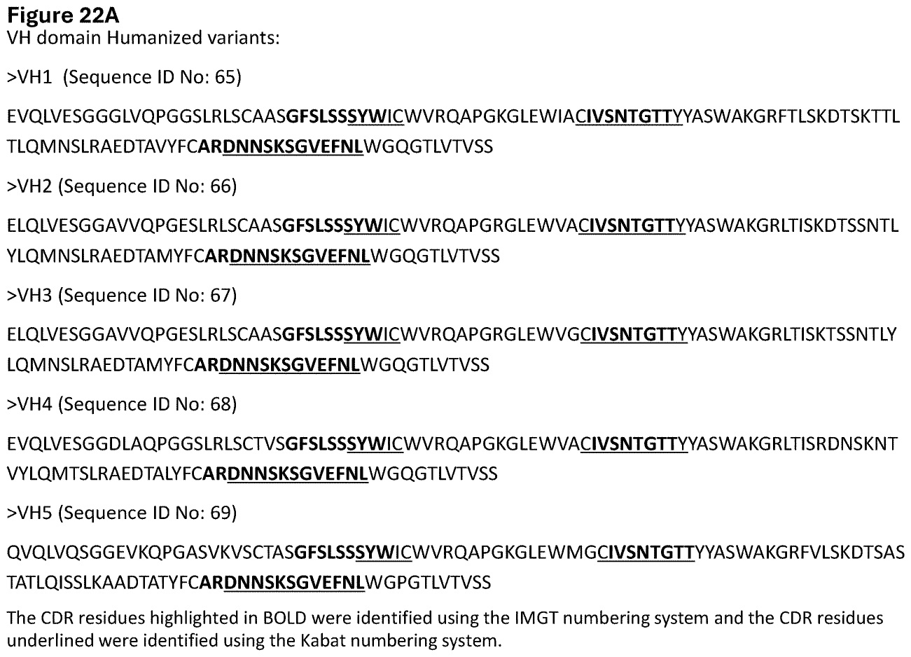

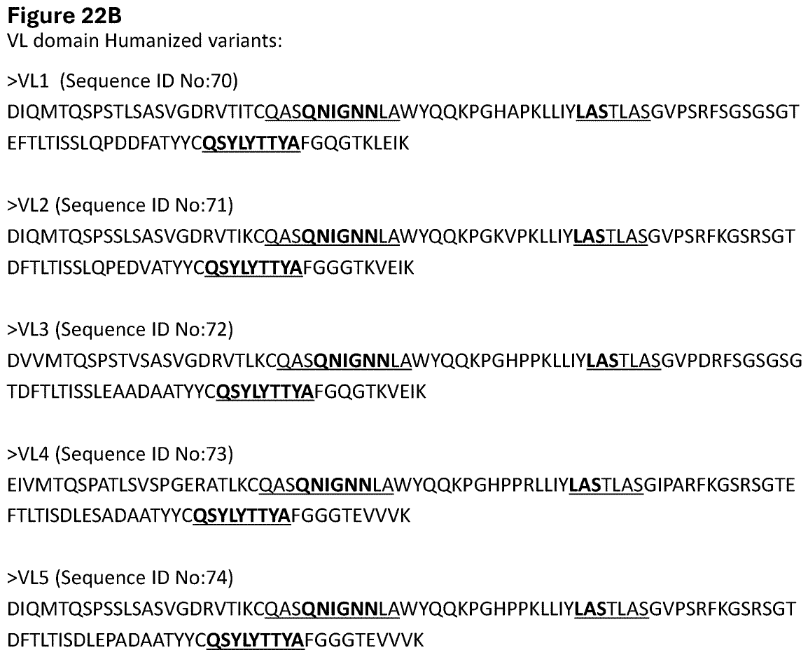

FIG. 22A illustrates VH domain humanized variants of the rabbit antibody 6D8 #28.

FIG. 22B illustrates VL domain humanized variants of the rabbit antibody 6D8 #28 domain

### EXAMPLES

### Example 1 Generation of Antibodies

### Expression and Purification of RAMP2 Variants

DNA coding for the amino acid sequence of several RAMP2 variants (see Table 1) were synthesised and cloned into the mammalian transient expression plasmid pETE V1 (Fusion antibodies' plasmids). RAMP2 variants were expressed using a HEK based transient expression system and the resulting antibody containing cell culture supernatants was clarified by centrifugation and filtration. RAMP2 variants were purified (using state-of-the-art AKTA chromatography equipment) from cell culture supernatants via affinity chromatography. Successfully purified products were dialysed/buffer exchanged into phosphate buffered saline solution. The purity of the RAMP2 variants were determined to be >95%, as judged by reducing and denaturing Sodium Dodecyl Sulphate Polyacrylamide gels. Bacterial endotoxin levels were determined using the Endosafe®-PTS system and Endosafe® PTS cartridges (Charles River Laboratories). Protein concentration was determined by measuring absorbance at 280 nm and calculated using the standard theoretical extinction coefficient (as determined by Expasy protoparm)

TABLE 1
Amino acid sequence of RAMP2 constructs:
(with signal peptide underlined)

>RAMP2 IMM V1
(Sequence ID No: 57)

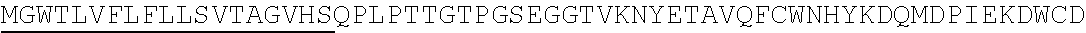

WAMISRPYSTLRDCLEHFAELEDLGFPNPLAERIIFETHQIHFANCSLVQPTFSDPPEDVGG
SHRRLDKIEDERNLHEDFVEMKTIQRCNKGEGSLSLLNCKEIRSQFEGFVKDIMLNKEEPKK
EINFEMQKGDQDPQIAAHLISEASSKSSSVLQWAKKGYYTMSNTLVTLENGKQLKVKROGFY
YIYAQVTFCSNOEPSSQAPFIASLCLKSSGGSERILLRAANARSSSKTCEQQSIHLGGVFEL

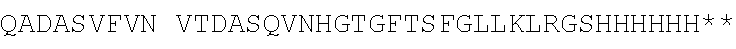

>RAMP2 Scr V1
(Sequence ID No: 58)

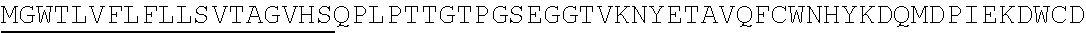

WAMISRPYSTLRDCLEHFAELEDLGFPNPLAERIIFETHQIHFANCSLVQPTFSDPPEDVGS
GAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKS
RWQQGNVFSCSVMHEALHNHYTQKSLSLSPGK*;
>RAMP2 IMM ECD V1
(Sequence ID No: 59)

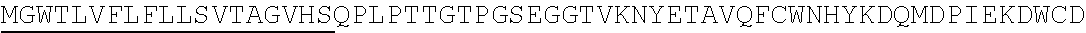

WAMISRPYSTLRDCLEHFAELFDLGEPNPLAERIIFETHQIHFANCSLVQPTFSDPPEDV
RGSHHHHHH
>RAMP2 IMM V2
(Sequence ID No: 60)

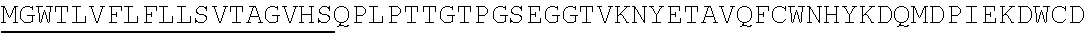

WAMISRPYSTLRDCLEHFAELFDLGEPNPLAERIIFETHQIHFANCSLVQPTFSDPPEDVGG
SPPELLGGPSVFIFPPKPKDTLMISRTPEVTCVVVDVSQDDPEVOFTWYINNEQVRTARPPL
REQQFNSTIRVVSTLPIAHQDWLRGKEFKCKVHNKALPAPIEKTISKARGQPLEPKVYTMGP
PREELSSRSVSLTCMINGFYPSDISVEWEKNGKAEDNYKTTPAVLDSDGSYFLYSKLSVPTS
EWQRGDVFTCSVMHEALHNHYTQKSISRSPGK**

### Expression and Purification of RAMP3 Variants

DNA coding for the amino acid sequence of several RAMP3 variants (see Table 2) were synthesised and cloned into the mammalian transient expression plasmid pETE V1 (Fusion antibodies plasmids). RAMP3 variants were expressed using a HEK based transient expression system and the resulting antibody containing cell culture supernatants was clarified by centrifugation and filtration. RAMP3 variants were purified (using state-of-the-art AKTA chromatography equipment) from cell culture supernatants via affinity chromatography. Successfully purified products were dialysed/buffer exchanged into phosphate buffered saline solution. The purity of the RAMP3 variants were determined to be >95%, as judged by reducing and denaturing Sodium Dodecyl Sulphate Polyacrylamide gels. Bacterial endotoxin levels were determined using the Endosafe®-PTS system and Endosafe® PTS cartridges (Charles River Laboratories). Protein concentration was determined by measuring absorbance at 280 nm and calculated using the standard theoretical extinction coefficient (as determined by Expasy protoparm).

TABLE 2
Amino acid sequence of RAMP3 constructs:
(with signal peptide underlined)
>RAMP3_IMM_V1
(Sequence ID No: 61)
MGWTLVFLFLLSVTAGVHSRAGGCNETGMLERLPLCGKAFADMMGKVDVWKWCNLSEFIVYYESFTNC
TEMEANVVGCYWPNPLAQGFITGIHRQFFSNCTVDRVHLEDPPDEVGGSHRRLDKIEDERNLHEDFVF
MKTIQRCNKGEGSLSLLNCKEIRSQFEGFVKDIMLNKEEPKKEINFEMQKGDQDPQIAAHLISEASSK
SSSVLQWAKKGYYTMSNTLVTLENGKQLKVKRQGFYYIYAQVTFCSNQEPSSQAPFIASLCLKSSGGS
ERILLRAANARSSSKTCEQQSIHLGGVFELQADASVFVN
VTDASQVNHGTGFTSFGLLKLRGSHHHHHH**
>RAMP3_Scr_V1
(Sequence ID No: 62)
MGWTLVELFLLSVTAGVHSRAGGCNETGMLERLPLCGKAFADMMGKVDVWKWCNLSEFIVYYESFTNC
TEMEANVVGCYWPNPLAQGFITGIHRQFFSNCTVDRVHLEDPPDEVGSGAPELLGGPSVFLFPPKPKD
TLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGK**
>RAMP3_IMM_ECD_V1
(Sequence ID No: 63)
MGWTLVFLFLLSVTAGVHSRAGGCNETGMLERLPLCGKAFADMMGKVDVWKWCNLSEFIVYYESFTNC
TEMEANVVGCYWPNPLAQGFITGIHRQFFSNCTVDRVHLEDPPDEVRGSHHHHHH**
>RAMP3_IMM_V2
(Sequence ID No: 64)
MGWTLVELFLLSVTAGVHSRAGGCNETGMLERLPLCGKAFADMMGKVDVWKWCNLSEFIVYYESFTNC
TEMEANVVGCYWPNPLAQGFITGIHRQFFSNCTVDRVHLEDPPDEVGGSPPELLGGPSVFIFPPKP
KDTLMISRTPEVTCVVVDVSQDDPEVQFTWYINNEQVRTARPPLREQQFNSTIRVVSTLPIA
HQDWLRGKEFKCKVHNKALPAPIEKTISKARGQPLEPKVYTMGPPREELSSRSVSLTCMING
FYPSDISVEWEKNGKAEDNYKTTPAVLDSDGSYFLYSKLSVPTSEWQRGDVFTCSVMHEALH
NHYTQKSISRSPGK**

### Immunization and Antibody Response

Three rabbits were immunized three times with RAMP2 Imm V2 (2.35 mg/ml, lot 030419) and three rabbits with RAMP3 Imm V2 (3.08 mg/ml, lot 030419) in three-week intervals using 200 μg antigen in complete Freund's adjuvant in first immunization and incomplete Freund's adjuvant in following immunizations. Animals were boosted three weeks after 3rd immunization by injecting half of the antigen dose in PBS intravenously and half of the dose in incomplete Freund's adjuvant subscapularly. Animals were sacrificed three days after final boost. Splenocytes were isolated from the spleens within 3 hours, and frozen for storage in liquid nitrogen.

The blood sera were obtained at Day 0, and 10 days after 2^{nd} or 3^{rd} immunization. The specific antibody titers were measured by ELISA, coating the plates (Nunc MaxiSorp, Thermo; 1 μg/ml in PBS) with on-target protein (RAMP2 Scr or RAMP3 Scr respectively). RAMP2 IMM V2 immunized rabbits were additionally tested on RAMP2 ECD (plate coated with 1 μg/ml in PBS, Lot 130219). Blood sera of all rabbits were also tested against irrelevant protein (plate coated with 2% BSA in PBS) for unspecific binding evaluation-no reaction with BSA was detected.

### Antibody Development by HybriFree Technology

### Panning of Splenocytes and Generation of Full Antibody Libraries

Panning experiment was performed on RAMP2 Scr or RAMP3 Scr (5 μg/ml) immobilized to Nunc™ MaxiSorp™ 96-well plates. Splenocytes (1×10⁴ cells per well) from RAMP2 Imm V2 immunized rabbits no. 53 and 54 or RAMP3 Imm V2 immunized rabbits no. 7 and 8 were used. After 45 min. incubation, wells were washed with PBS to remove the unbound cells. RNA was isolated and cDNA was synthesized using SuperScript® IV First-Strand Synthesis System for RT-PCR (Invitrogen) and used for VH and VL amplification. Totally 24 reactions with both target antigens were performed.

The proprietary VH and VL primers were designed using rabbit sequences stored at IMGT® [http://www.imgt.org/IMGTrepertoire/Accessed August 2015] and literature data:
Kodangatti S. et al. The functional repertoire of rabbit antibodies and antibody discovery via next-generation sequencing. MAbs. 2014 May-June; 6(3):628-36.
Lavinder J J. et al. Systematic characterization and comparative analysis of the rabbit immunoglobulin repertoire. PLoS One. 2014 Jun. 30; 9(6).
Kivi G. et al. HybriFree: a robust and rapid method for the development of monoclonal antibodies from different host species. 2016 January.

Amplified VH and VL were purified and altogether 20 cloning reactions per target were performed for cloning into rabbit IgG-κ1 encoding two-cassette expression plasmid by using Ligase Independent Cloning (LIC) method. Resulting antibody library pools were grown in E. coli strain DH5a. Plasmid DNA was purified and transfected into CHOEBNALT85-1E9 cells for transient production of antibodies in serum-free media for 48 hours in 24-well format. Cell culture supernatants were tested for RAMP2 Scr and RAMP2 ECD or RAMP3 Scr binding in ELISA (wells coated at 1 μg/ml in PBS).

### Results:

17 ELISA positive pools against RAMP2 (tested on Scr and ECD protein) from rabbit no. 53 and no. 54 were identified.
16 ELISA positive pools against RAMP3 (tested on Scr protein) from rabbit no. 7 and no. 8 were identified.

### Expression of Full Antibody Library Clones, VH/VL Sequence Verification

8 specific ELISA positive pools against both targets were selected for single clone isolation. Single clones were selected in LB-Amp solid medium, colonies were grown in liquid medium o/n on shaker at 37° C. in 96-well microtiter plates (2-4 96-well plates per target), plasmid DNA was isolated and transfected into CHOEBNALT85-1E9 cells for antibody transient production. 48-72 hours after transfection supernatants were analyzed by ELISA on RAMP2 Scr or RAMP3 Scr respectively.

ELISA positive rlgG-K clones to RAMP2 Scr and RAMP3 Scr were identified from each selected pool, 17 RAMP2 specific and 32 RAMP3 specific clones were analyzed by sequencing.

### Results:

7 unique anti-RAMP2 antibody clones were identified (1A7, 1H6, 1H7, 1A10, 2A6, 2A8, 2H3).
7 unique anti-RAMP3 antibody clones were identified (3A5, 3A12, 4C10, 5H9, 6B8, 6C2, 6D8).

Sequence alignments and phylogenetic trees of isolated and selected antibody clones are shown on FIGS. 1 and 2.

Antibodies are clustered, identical or similar VH and VL are grouped, and CDRs are marked with blue on consensus sequence. For better overview, only final selection of clones with different CDR-s are shown in alignment (at least 5 amino acid difference in CDR-s). Comparison of anti-RAMP2 and anti-RAMP3 antibodies heavy and light chain CDR-s are shown in FIGS. 3 and 4 respectively.

### Expression of Selected Clones in 24-Well Format

Isolated antibody clones were subcloned, plasmid miniprep DNA was isolated and transfected into CHOEBNALT85-1E9 cells for antibody transient production in 24-well format. 48 hours after transfection specific binding from supernatants on screening antigen (RAMP2 Scr and RAMP2 ECD or RAMP3 Scr) was confirmed by ELISA, and full heavy and light chain sequences were verified by sequencing.

### Results:

all 7 isolated and selected anti-RAMP2 antibody clones (1A7, 1H6, 1H7, 1A10, 2A6, 2A8, 2H3) gave specific reaction on RAMP2 (ELISA signals on Scr and ECD were comparable) and contained unique VH/VL sequence after subcloning.
all 7 isolated and selected anti-RAMP3 antibody clones (3A5, 3A12, 4C10, 5H9, 6B8, 6C2, 6D8) gave specific reaction on RAMP3 and contained unique VH/VL sequence after subcloning.

### 6-Well Production of RAMP2 and RAMP3 Specific Rabbit IgG-κ1 Antibodies

14 developed rabbit IgG-κ1 antibody clones (7 clones against RAMP2 and 7 clones against RAMP3) were transfected into CHOEBNALT85-1E9 cells for transient production in 6-well format (2 ml of media). 10 days after transfection, cells were removed by centrifugation (300 rcf for 5 minutes), 1.5 mL of each supernatant was aliquoted into screw top vials in sterile conditions.

Produced antibody supernatants were tested by ELISA on RAMP2 ECD or RAMP3 Scr coated plates.

### Results:

Titers of all 7 isolated anti-RAMP2 antibody clones were <8 ng/ml.
Anti-RAMP3 antibody clone titers varied from 8-500 ng/ml.

Immuno modules (Thermo Scientific™) were coated with RAMP2 ECD or RAMP3 Scr, 1.0 μg/ml in PBS, 100 μl/well, 20 h at 4° C. Supernatants of anti-RAMP2 and anti-RAMP3 rabbit IgG-κ1 antibody clones were transfered into wells at different concentrations (8-500 ng/ml; 100 μL/well, in PBS-0.05% Tween 20-1% BSA) and the plates were incubated for 60 min. on a shaker at room temperature (RT). Goat anti-rabbit-IgG HRP conjugate (Invitrogen; 1:20 000 in 1% BSA-PBS-0.05% Tween 20; 100 μL/well) was used as secondary reagent in direct ELISA (incubation time 60 min. at RT). To visualize, TMB substrate was added for 10 min. and stopped with 0.5 M H₂SO₄. Between every incubation step the wells were washed 4 times with PBS-Tween 20.

### Example 2 In Vitro Characterisation of Generated Antibodies

### Materials and Methods

### Cell Culture

Human umbilical vein endothelial cells (HUVECs), purchased from Lonza (Paris, France) were cultured in EBM-2 medium (Lonza) supplemented with hydrocortisone (1 μg/ml), bovine brain extract (12 μg/ml), epidermal growth factor (10 ng/ml) and 2% fetal bovine serum (FBS; Life Technologies) in humidified incubator at 37° C. with air/5% CO₂. HUVECs were tested negative for mycoplasma and cultured until the fifth passage. U87MG, HEP3B, DU145, PC3, LnCAP, O786, JHH6, SKHEP1 and Caki-1 were obtained from the American Type Culture Collection. Cell lines were maintained in T-25 culture flasks in RPMI 1640 supplemented with 10% FBS (#26140079, Gibco FBS qualified USA origin, Life Technologies), 2 mM glutamine (PAA Laboratories), 100 units/ml penicillin and 100 μg/ml streptomycin (PAA Laboratories) at 37° C. in a humidified 5% CO₂ atmosphere. Cell lines with a passage number in the range between 5 and 40 were used and were maintained in culture for a maximum of 6 weeks. Cell counting was performed with a hemacytometer.

### HUVEC Cell Angiogenesis Assay

HUVECs cells (7×10⁴) were pre-incubated with 60 μg/mL RAMP2 Ab clones (1A7_#1; 1H6_#4; 1H7_#5; 1A10_#7; 2A6_#9; 2A8_#12 and 2H3_#13), or 60 μg/mL RAMP3 Ab clones (3A5_#15; 3A12_#17; 4C10_#19; H9_#22; 6B8_#24; 6C2_#25 and 6D8_#28), or 60 μg/mL RAMP2 (B-5) sc-365240 obtained from Santa Cruz Biotechnology, Inc (Texas, USA) or 60 μg/mL RAMP3 (G-1) sc-365313 obtained from Santa Cruz Biotechnology, Inc (Texas, USA) for 30 min before to be seeded on Matrigel-precoated wells with 300 μl of 8.5 mg/ml solution of BD Matrigel (BD Biosciences). For this tube-formation assay, medium containing 0.5% FBS was supplemented with 2.10-7 M adrenomedullin (AM) (Bachem, Bubendorf, Switzerland) in absence or presence of 60 μg/mL RAMPs ab. The plates were incubated for 5 h at 37° C., and fixed with methanol-free 4% paraformaldehyde and microscopic images were collected for analysis of junction and node formation. For the quantitative image analysis, the inventors evaluated the Total Branching Points, defined as parts of the skeleton where three or more tubes converge and Total Loops defined as the area of the background enclosed (or almost) by the tubular structure, using the WimTube analysis developed by WIMASIS Image analysis (www.wimasis.com).

### Cell Invasion Assays

Invasiveness was measured using Matrigel invasion chambers (24-well, BD Biosciences). Membranes (8 μm) were coated with Matrigel (50 μg, BD Biosciences) and placed in the lower chamber containing 0.6 ml of RPMI 1640 medium supplemented with 1.10-7 M adrenomedullin (AM) (Bachem, Bubendorf, Switzerland) as chemoattractant. U87MG or Caki-1 cells at 1.10⁵ cells were pre-incubated with 60 μg/mL RAMP2 Ab clone (1H6_#4), or 60 μg/mL RAMP3 Ab clones (3A5_#15; 3A12_#17; 4C10_#19; 6B8_#24; 6C2_#25 and 6D8_#28) for 120 min before to be seeded on the inserts suspended in 0.3 ml of serum-free RPMI 1640. After incubation for 24 h with or without RAMPs Ab, the upper surface of the filter was scraped to remove non-invasive cells. Invasive cells were fixed and stained with a Diff Quik Detection Kit (Sigma-Aldrich, Saint-Louis, USA). The average number of invading cells per field was assessed by counting 9 random fields under a light microscope (400×).

### Cell Proliferation Assay

Cell proliferation was determined using the MTT assay (3-[4,5-dimethylthiazol-2-yl]-2,5-diphenyltetrazolium bromide). The conversion of yellow water-soluble tetrazolium MTT into purple insoluble formazan is catalyzed by mitochondrial dehydrogenases and can be used to estimate the number of viable cells. U87MG, HEP3B, DU145, PC3, LnCAP, O786, JHH6, SKHEP1 and Caki-1 cells were seeded in 96-well tissue culture plates at a density of 3×10³ cells/well and 24 h later, cells were pre-incubated with 60 μg/mL RAMP2 Ab clone (1H6_#4), or 60 μg/mL RAMP3 Ab clones (3A5_#15; 3A12_#17; 4C10_#19; 6B8_#24; 6C2_#25 and 6D8_#28) for 120 min, after RPMI 1640 supplemented with 1.10-7 M AM (Bachem, Bubendorf, Switzerland) or 10% SFB was added in each well. After drug exposure for 48 h, cells were incubated with 0.4 mg/ml MTT for 4 h at 37° C. After incubation, the supernatant was discarded, the cell pellet was resuspended in 0.1 mL DMSO, and absorbance was measured at 560 nm using a microplate reader MultiSKan EX (Thermo Scientific, France). Wells with RPMI-1640 were used as negative controls. Experiments were performed in triplicate or quadruplicate and were independently performed at least three times, unless otherwise indicated. Results are expressed as the mean and SEM of at least three independent experiments, unless otherwise indicated.

### Statistical Analysis

Results are expressed as mean±SEM of at least three independent experiments. Statistical analysis was performed by two-way ANOVA followed by Bonferroni a posteriori test employing Prism 9.2 for MS Windows software (Graph Pad Software). A p-value of 0.05 or less was considered statistically significant.

### Results

### HUVEC Cell Angiogenesis Assay

To explore the ability of RAMPs Ab clones to inhibit endothelial cell angiogenesis induced by AM, the inventors measured its ability to inhibit the formation of vascular structures in an in vitro Matrigel assay of HUVEC cells in the presence of AM. In the presence of 0.5% FBS, HUVECs remained round and isolated on the Matrigel bed, with no cell spreading. These data demonstrate that 5 h-treatment with 9 out of 14 evaluate RAMP Ab clone blocks (cut off >50%) the morphogenetic effects induced by AM in the HUVEC differentiation into vascular structures, which would be necessary in vivo for the sprouting of endothelial cells and tube formation. On the other hand, HUVEC pre-treatment with two available anti-RAMP2 and anti-RAMP3 commercial antibodies developed by Santa Cruz Biotechnology, Inc; were not able to block the AM pro-angiogenic effects evaluated by the assessment of the total branching points and total loops of HUVEC structures (FIG. 5).

### Cell Invasion Assay

To test whether RAMP2 and RAMP3 Ab clones could affect glioblastoma and renal carcinoma cells motility, U87MG and Caki-1 cells were incubated for 2 hr with the RAMPs Abs in a Boyden chamber with 0.1 μM AM. The number of cells that migrated to the lower surface of the transwell membranes was increased in the AM group respect to control group (FIG. 2). Preincubation of either 1H6_4, 3A12_17 and 4C10_19 Abs in U87MG or 3A12_17 and 6C2 25 in Caki-1 at 60 μg/ml each for 120 min caused a significant inhibition of invasion respect to control cells in presence of AM after 24 h of incubation at 37° C., indicating that this response to AM was blocked for the mentioned Abs clones (FIG. 6).

### Cell Proliferation Assay

In vitro tumor cell proliferation was evaluated in a panel of cancer cell lines belonging to prostate model (DU145 and PCC3), hepatocarcinoma (SKHEP1, JHH6 and HEP3B) and renal cell carcinoma Caki-1. Cells were treated with the different RAMPs Ab clones at at 60 μg/ml in the presence or not of AM and SFB during 48 h. Under the evaluated conditions, the inventors didn't observed inhibition of the cell proliferation for the treatment with the RAMPs Ab clones (FIG. 7).

### Example 3

### Xenografts Model

The experimental protocol was approved under the number APAFIS #33619-2021102212389984 v3 by the French Ministry of Higher Education and Scientific Research. Six- to eight-week-old male athymic NMRI nude mice were injected subcutaneously in the right lateral flank with 0.5×10⁶ U87MG cells in 200 μl phosphate buffered saline (PBS). Two days after subcutaneous cells injection, animals were randomly divided into 4 groups (5 mice in each group) and treated with one of the following:
Vehicle (PBS, intra-peritoneal (ip), continuous)
3A12_#17 (anti-RAMP3), at 16.5 mg/kg (~330 μg/mice), ip, 3qw (t.i.w)
6B8_#24 (anti-RAMP3), at 16.5 mg/kg (~330 μg/mice), ip, 3qw (t.i.w)
6D8_#28 (anti-RAMP3), at 16.5 mg/kg (~330 μg/mice), ip, 3qw (t.i.w)

Mice were sacrificed upon development of apparent symptoms, such as lethargy or hunched posture or volume reached 1300-1400 mm³. The tumor size measurements were recorded three times a week (Mon, Wed and Fri) from treatment start until the end of the study.

When possible, control and Abs-treated mice were sacrificed 4 hours after the last treatment. Tumors were obtained by surgery and divided immediately into 2 parts. One part was immediately snap frozen and stored at −80° C., and the second part was fixed in 10% neutral buffered formalin (NBF) overnight at 4° C. then subjected to routine paraffin embedding. In addition, adjacent normal tissue was collected, immediately snap frozen and stored at −80° C.

### Statistical Analysis

Mean and standard error were determined for tumor volume and body weights for all experimental groups. Statistical analysis was performed with GraphPad Prism version 8.0 software (GraphPad software, Inc., La Jolla, CA, USA). ANOVA followed by Dunnet's multiple comparison test was performed to evaluate if there are any statistically significant differences between treated and control groups.

### Results

Evaluation of the in vivo efficacy of 3 RAMPs mAbs in subcutaneous murine glioblastoma U87MG tumors.

The U87MG cell line, a model of human glioblastoma cell line with epithelial morphology was selected to evaluate 3 RAMPs mAbs (3A12_#17, 6B8_#24 and 6D8_#28) as single agent.

RAMPs Abs were administered ip at 16.5 mg/kg 3 times per week from 48 h-after cell injection until animal sacrifice. Tumor size was recorded three times a week (Mon, Wed and Fri) and animals were sacrificed when volume reached 1300-1400 mm³ for ethical reasons. Tumor growth in animals treated with Abs 3A12_#17 or 6B8_#24 were similar to control group. On the other hand, animals treated with 6D8_#28 showed a significant reduction of tumor growth at day 23 respect to control group (FIG. 8).

Abs-treated mice did not show sign of toxicity throughout the experiment, including variations in body weight with respect to control group (FIG. 9).

Analysis of Tumour tissue

### Necrosis

Samples of tumour tissue taken from the animals showed further evidence of prominent necrosis throughout the tumour tissue. Paraffin-fixed tissue slides taken from each individual animal were stained with anti-Caspase 3 antibody and imaged. (the method was as described for the apoptosis analysis as described below but with necrotic cells judged by eye).

The control animals treated with PBS only showed a small proportion of necrotic cells distributed evenly across the tissue section.

The tissue sections from the animals treated with the antibodies each showed large sharply bordered areas of necrosis to a greater or lesser degree. In addition throughout the otherwise healthy tumour tissue the cells associated with the vascular system showed significant necrosis. A series of representative images for each of the treatment groups is shown below, necrotic cells are stained red/brown.

FIG. 10 illustrates a pair of representative microscopic images of a U87MG tumour tissue section stained for necrosis (brown=necrotic cells)—PBS control group. FIG. 11 illustrates Representative microscopic images of U87MG tumour tissue sections stained for necrosis (brown=necrotic cell)—RAMP3 #28 treated group. Left-large defined area of progressively increasing necrosis. Right-close up of vascular associated cells in otherwise healthy tissue showing necrotic epithelial cells. FIG. 12 shows representative microscopic images of U87MG tumour tissue sections stained for necrosis (brown=necrotic cell)—RAMP3 6B8 #24 treated group. Left-large defined area of progressively increasing necrosis. Right-close up of vascular associated cells in otherwise healthy tissue showing necrotic epithelial cells. FIG. 13 Representative microscopic images of U87MG tumour tissue sections stained for necrosis (brown=necrotic cell)—RAMP3 3A12 #17 treated group. Left-large area of progressively increasing necrosis, less firmly bordered than other treatment groups. Right-close up of vascular associated cells in otherwise healthy tissue showing necrotic epithelial cells There were some differences observed in the pattern of necrosis between the 3 antibodies. RAMP3 6D8 #28 treated tissue showed the greatest degree of large fully necrotic areas with significant necrosis in vascular cells throughout the healthy tissue, but to a greater degree adjacent to the continuous necrotic area. RAMP3 6B8 #24 showed somewhat smaller areas of continuous necrosis, but appeared to show a greater degree of spread in the vascular necrosis than 6D8 #28. RAMP3 3A12 #17 showed a lesser degree of necrotic areas, and similarly less vascular necrosis than the other 2 treatment groups. The results are summarized in Table 1 below:

TABLE 1
Summary of analysis of necrosis within U87MG tumours treated with antibodies

| Treatment | Observed | | Vascular | |
|---|---|---|---|---|
| Group | Necrosis | Description | Necrosis | Description |
| Control (PBS | +/− | Necrotic cells | — | No evidence of specific |
| vehicle only) | | observed at a low | | necrosis associated with |
| | | level throughout | | vasculature |
| | | tumour | | |
| RAMP3 Ab | +++ | Large, discrete | ++ | Vasculature in living |
| 6D8#28 | | areas of tumour | | tissue adjacent to |
| | | slide fully necrotic | | necrotic areas show |
| | | | | substantial necrosis |
| RAMP3 Ab | ++ | Large discrete areas | +++ | Necrotic vasculature |
| 6B8#24 | | of tumour slide fully | | throughout otherwise |
| | | necrotic | | healthy tumour tissue |
| | | | | highly necrotic |
| RAMP3 Ab | + | Smaller areas of | + | Evidence of vascular |
| 3A12#17 | | necrosis observed | | necrosis observed |
| | | throughout tumour | | throughout tumour |

(The scoring estimates are based on the aggregates of n = 5 animals in each group. +++ = 70-90% ++ = 50-69%, + = 30-49%, +\− = 5-29%)

### Apoptosis

An additional set of fixed tissue sections were further examined for active apoptotic activity within cells. Briefly, apoptosis level was evaluated on formalin-fixed paraffin-embedded U87 xenografts and kidney tissue samples and immunochemical analysis was conducted using the Vectastain Elite ABC Universal kit (Vector Laboratories, Burlingame, CA, USA) as previously described (Berenguer et al., Clin, Cancer Res. 2013, 19, 6138-6150). A rabbit monoclonal antibody (purified Ig G) generated against active form of caspase-3 (1:400; Cell Signaling, France) was used. Sections of paraffin-embedded samples (4 micron) were tested for the presence of the active form of caspase-3 after heat-induced antigen retrieval in citrate buffer (pH 6) at 97° C. for 40 min. Incubation was maintained overnight at 4° C. and followed by a second layer containing biotinylated anti-rabbit antiserum (Histostain plus, Zymed). The next treatment was with the avidin-biotin peroxidase complex kit (Histostain plus, Zymed). Note that endogen biotin and endogen peroxidases were respectively neutralized by a biotin-blocking system (Dako) and 3% H2O2.

Overall the pattern of staining was very similar to the necrosis staining described above with large blocks of apoptotic and dead cells as well as vascular necrosis throughout the tissue section. Again RAMP3 6D8 #28 shows the largest blocks of stained cells, with significant vascular staining adjacent to the large blocks. RAMP3 6B8 #24 shows slightly smaller blocks, with a greater distribution of apoptosis in vascular cells throughout the tumour section and RAMP 3A12 #17 showing a lower level of apoptotic staining overall. A series of representative images from each of the 4 treatment groups is given in FIGS. 14-21. FIG. 14 shows a representative microscopic image of a U87MG tumour tissue section stained for caspase 3 (brown=apoptotic cells)—PBS control group. FIG. 15 shows representative microscopic images of U87MG tumour tissue sections stained for caspase-3 (brown=apoptotic cell)—RAMP3 6D8 #28 treated group. Left-large defined area of progressively increasing apoptosis and cell death. Right-close up of vascular associated cells in otherwise healthy tissue showing apoptotic epithelial cells. FIG. 16 shows representative microscopic images of U87MG tumour tissue sections stained for caspase-3 (brown=apoptotic cell)—RAMP3 6B8 #24 treated group. Left-large defined area of progressively increasing apoptosis and cell death. Right-close up of vascular associated cells in otherwise healthy tissue showing apoptotic epithelial cells. FIG. 17 shows representative microscopic images of U87MG tumour tissue sections stained for caspase-3 (brown=apoptotic cell)—RAMP3 3A12 #17 treated group. Left-large area of progressively increasing apototic cells, less firmly bordered than other treatment groups. Right-close up of vascular associated cells in otherwise healthy tissue showing apoptotic epithelial cells. FIGS. 18 to 21 show lower magnification images of tissue sections taken from the U87MG xenograft from the mouse treated with PBS vehicle control Ab6D8 #28, Ab6B8 #24 and Ab3A12 #17 respectively.

As with the necrotic staining above, RAMP3 6D8 #28 appears to exert the strongest effect on the apoptosis of cells within the tumour. Table 2 summarizes the caspase-3 staining:

TABLE 2

| | Observed | | Vascular | |
|---|---|---|---|---|
| Treatment | Caspase-3 | | Caspase-3 | |
| Group | activation | Description | activation | Description |
| Control (PBS | +/− | Caspase 3 +ve cells | +/− | Some caspase 3 +ve |
| vehicle only) | | observed distributed | | cells observed in |
| | | throughout tumour | | vascular cells, does |
| | | tissue (5-25%) | | not appear |
| | | | | concentrated |
| RAMP3 Ab | +++ | Significant sharply | +++ | Vasculature |
| 6D8#28 | | bounded blocks of | | throughout unstained |
| | | tissue show caspase | | regions of tissue, |
| | | 3 staining (70-90% | | show substantial |
| | | estimate*) | | staining |
| RAMP3 Ab | +++ | Significant sharply | ++ | Vasculature |
| 6B8#24 | | bounded blocks of | | throughout unstained |
| | | tissue show caspase | | regions of tissue, |
| | | 3 staining (70-90% | | show some staining |
| | | estimate*) | | |
| RAMP3 Ab | +++ | Significant sharply | +++ | Vasculature |
| 3A12#17 | | bounded blocks of | | throughout unstained |
| | | tissue show caspase | | regions of tissue, |
| | | 3 staining (70-90% | | show substantial |
| | | estimate*) | | staining |

(The scoring estimates are based on the aggregates of n = 5 animals in each group. +++ = 70-90% ++ = 50-69%, + = 30-49%, +/− = 5-29%)

### Off Target Effects—Kidney Vasculature

In addition to the detailed analysis of tumour tissue sections, samples were also taken of highly vascularized regions of kidney tissue within the treated animals and analysed for the presence of apoptosis as shown above. Whilst apoptotic cells could be seen distributed relatively evenly throughout the tissue sections, no observable difference in pattern or frequency could be noted between the control group and each of the 3 treatment groups in the study. This data helps to support the treatment as being highly specific and safe to the normal vascular function of the treated animals. The observations are summarized in Table 3 below.

TABLE 3

| Treatment | Necrosis/ | | Vascular | |
|---|---|---|---|---|
| Group | Apoptosis | Description | Necrosis | Description |
| Control (PBS | +/− | Some necrotic cells | +/− | Some necrotic |
| vehicle only) | | observed | | vascular cells |
| | | distributed | | observed |
| | | throughout tissue | | throughout tissue |
| RAMP3 | +/− | Some necrotic cells | +/− | Some necrotic |
| Ab6D8#28 | | observed | | vascular cells |
| | | distributed | | observed |
| | | throughout tissue | | throughout tissue |
| RAMP3 | +/− | Some necrotic cells | +/− | Some necrotic |
| Ab6B8#24 | | observed | | vascular cells |
| | | distributed | | observed |
| | | throughout tissue | | throughout tissue |
| RAMP3 | +/− | Some necrotic cells | +/− | Some necrotic |
| Ab3A12#17 | | observed | | vascular cells |
| | | distributed | | observed |
| | | throughout tissue | | throughout tissue |

Our findings showed that the antibody against RAMP3 (clone 6D8_#28) decreased U87-tumor growth respect to control group, without displaying any toxicity effect during the period of treatment. Furthermore each of the treated animals showed substantial areas of necrosis, and active apoptosis within the tumour interior and throughout the tumour vasculature, not reflected in the overall tumour volume. RAMP3 clone 6D8 #28 again showed the greatest effect in regards to this analysis.

The inventors' findings show that each of the Abs 3A12_#17, 6B8_6B8 #24, and 6D8_#28 antibodies cause necrosis of tumours with respect to animals treated with the control vehicle without displaying any toxicity effect during the period of treatment and, moreover, the 6D8_#28 antibody decreased U87-tumor growth with respect to control group.

### Example 4 Humanization Process for Anti-RAMP3 Antibody 6D8 #28

### Stage 1: Humanization Design

The variable region sequences of rabbit antibody 6D8 #28 were used to generate humanized antibody designs. Variable chains were humanized by grafting the CDR sequences into suitable mature human antibody sequences. Recipient human frameworks were selected for key residues identified by our proprietary platform that are critical to protein structure, stability and function. These acceptor sequences have all come from mature Human IgG from a human source and not from phage display or other technologies. As a result, the humanized sequences are expected to be non-immunogenic and retain the canonical structure of the CDR-loops. Using antibody numbering systems from IMGT and Kabat, the CDRs were identified. These two numbering systems identify different residues of the rabbit antibody as belonging to the CDR, and Fusion's Humanization technology uses a combined IMGT/Kabat CDR sequence for optimal retention of CDR-loop conformation. Grafting of combined Kabat/IMGT CDRs from the rabbit 6D8 #28 antibody into the acceptor framework sequences resulted in the humanized variants. The humanized sequences were screened for liabilities including T-cell epitopes, glycosylation and deamidation. Five (5) variants were designed for each variable chain using different human donor sequences. These are shown in FIGS. 22A and 22B. This produced a matrix of twenty-five (25) human antibodies for expression. 3-Dimensional structures of the designed humanized antibodies were also generated using protein structure prediction software. The software utilizes algorithms that have been developed to combine comparative sequences to databases of canonical loop conformations, along with ab initio structure predictions. This builds accurate, full 3D atomic models of proteins with previously unknown structures.

Stage 2: The DNA that would code for the expressed humanized variants was optimized for codon usage, and synthesized and cloned into the pETE expression vector.

Stage 3: CHO cells were adapted to serum-free conditions and at a suitable cell density, separate cultures were transfected with the expression vector for each of the twenty-five (25) human variants in combinations of heavy chain and light chain pairs. Following this the antibodies were purified by affinity chromatography with a target yield of >100 μg. Upon successful purification, the antibody was dialyzed into PBS before QC analysis. The antibody was analyzed for purity and size by reducing and denaturing SDS-PAGE analysis and quantified by UV spectroscopy.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. An antibody molecule which binds RAMP2 or RAMP3 and inhibits adrenomedullin activity,
wherein the antibody molecule comprises at least one, for example two or three, of the CDRs of a VL chain and/or at least one, for example two or three, of the CDRs of a VH chain, wherein
(i) said VL chain has the amino acid sequence shown as Seq ID No: 53 and said VH chain has the amino acid sequence shown as Seq ID No: 54, or
(ii) said VL chain has the amino acid sequence shown as Seq ID No: 33 and said VH chain has the amino acid sequence shown as Seq ID No: 34; or
(iii) said VL chain has the amino acid sequence shown as Seq ID No: 45 and said VH chain has the amino acid sequence shown as Seq ID No: 46; or
(iv) said VL chain has the amino acid sequence shown as Seq ID No: 1 and said VH chain has the amino acid sequence shown as Seq ID No: 2, or
(v) said VL chain has the amino acid sequence shown as Seq ID No: 5 and said VH chain has the amino acid sequence shown as Seq ID No: 6; or
(vi) said VL chain has the amino acid sequence shown as Seq ID No: 9 and said VH chain has the amino acid sequence shown as Seq ID No: 10, or
(vii) said VL chain has the amino acid sequence shown as Seq ID No: 13 and said VH chain has the amino acid sequence shown as Seq ID No: 14; or
(viii) said VL chain has the amino acid sequence shown as Seq ID No: 17 and said VH chain has the amino acid sequence shown as Seq ID No: 18; or
(ix) said VL chain has the amino acid sequence shown as Seq ID No: 21 and said VH chain has the amino acid sequence shown as Seq ID No: 22, or
(x) said VL chain has the amino acid sequence shown as Seq ID No: 25 and said VH chain has the amino acid sequence shown as Seq ID No: 26; or
(xi) said VL chain has the amino acid sequence shown as Seq ID No: 29 and said VH chain has the amino acid sequence shown as Seq ID No: 30; or
(xii) said VL chain has the amino acid sequence shown as Seq ID No: 37 and said VH chain has the amino acid sequence shown as Seq ID No: 38; or
(xiii) said VL chain has the amino acid sequence shown as Seq ID No: 41 and said VH chain has the amino acid sequence shown as Seq ID No: 42; or
(xiv) said VL chain has the amino acid sequence shown as Seq ID No: 49 and said VH chain has the amino acid sequence shown as Seq ID No: 50.

2. The antibody molecule according to claim 1, wherein 3 or less amino acid substitutions, have been made in at least one of said CDRs.

3. The antibody molecule according to claim 1, wherein the antibody molecule comprises at least one CDR of the VL chain and at least one CDR of the VH chain.

4. The antibody molecule according to claim 3, wherein the antibody molecule comprises all 3 CDRs of the VL chain and all 3 CDRs of the VH chain.

5. The antibody molecule according to claim 4, wherein the antibody molecule comprises all 3 CDRs of the VL chain having the amino acid sequence shown as Seq ID No: 53 and all 3 CDRs of the VH chain having the amino acid sequence shown as Seq ID No: 54.

6. The antibody molecule according to claim 5, wherein said VL chain has the amino acid sequence shown as Seq ID No: 53 and said VH chain has the amino acid sequence shown as Seq ID No: 54.

7. The antibody molecule according to claim 4, wherein the antibody molecule comprises all 3 CDRs of the VL chain having the amino acid sequence shown as Seq ID No: 33 and all 3 CDRs of the VH chain having the amino acid sequence shown as Seq ID No: 34.

8. The antibody molecule according to claim 7, wherein said VL chain has the amino acid sequence shown as Seq ID No: 33 and said VH chain has the amino acid sequence shown as Seq ID No: 34.

9. The antibody molecule according to claim 4, wherein the antibody molecule comprises all 3 CDRs of the VL chain having the amino acid sequence shown as Seq ID No: 45 and all 3 CDRs of the VH chain having the amino acid sequence shown as Seq ID No: 46.

10. The antibody molecule according to claim 9, wherein said VL chain has the amino acid sequence shown as Seq ID No: 45 and said VH chain has the amino acid sequence shown as Seq ID No: 46.

11. An antibody molecule according to claim 5, having a heavy chain variable sequence selected from the VH amino acid sequences shown as Sequence ID Nos: 65, 66, 67, 68, and 69, and a light chain variable sequence selected from the VL amino acid sequences shown as Sequence ID Nos: 70, 71, 72, 73, and 74.

12. An antibody molecule which binds RAMP2 or RAMP3 and inhibits adrenomedullin activity.

13. The antibody molecule according to claim 1, wherein the antibody molecule induces or accelerates necrosis of tumour cells.

14. A nucleic acid encoding the antibody molecule according to claim 1.

15. A pharmaceutical composition comprising the antibody molecule according to claim 1 or a nucleic acid encoding the antibody molecule.

16. A method of inducing or accelerating necrosis in a tumour in a patient, said method comprising administration of the antibody molecule according to claim 1, a nucleic acid encoding the antibody molecule, or a pharmaceutical composition comprising the antibody molecule according to claim 1or a nucleic acid encoding an antibody molecule to said patient.

17. A method of treating a condition associated with angiogenesis in a patient in need of treatment thereof, said method comprising administration of the antibody molecule according to claim 1, a nucleic acid encoding the antibody molecule, or a pharmaceutical composition comprising the antibody molecule according to claim 1or a nucleic acid encoding the antibody molecule to said patient.

18. The antibody molecule according to claim 1 or a nucleic acid encoding the antibody molecule for use in medicine.

19. The antibody molecule according to claim 1 or a nucleic acid encoding an antibody molecule for use in the treatment of a condition associated with angiogenesis.

20. The antibody molecule or nucleic acid according to claim 19, wherein said condition is a condition associated with aberrant adrenomedullin activity.

21. The method according to claim 17, the antibody molecule which binds RAMP2 or RAMP3 and inhibits adrenomedullin activity, or the nucleic acid encoding an antibody molecule, wherein the condition is cancer.

22. The method according to claim 21, wherein the cancer is glioblastoma, renal cancer, prostate cancer, colon cancer, lung cancer, mesothelioma, or pheochromocytoma.

23. The method, antibody molecule, or nucleic acid according to claim 22, wherein the cancer is glioblastoma.

24. The method, antibody molecule, or nucleic acid according to claim 22, wherein the cancer is renal cancer.

25. A method of producing an antibody molecule capable of inhibiting angiogenesis, said method comprising expressing the nucleic acid according to claim 14 in a host cell and isolating said antibody molecule from said cell.


SEQUENCE LISTING (78 sequences)

SEQ ID NO: 1 - protein, 113 aa, Oryctolagus cuniculus
  BINDING 27..32: note = Complementarity-determining region
  BINDING 50..52: note = Complementarity-determining region
  BINDING 89..103: note = Complementarity-determining region
APVLTQTPASVEAAVGGTVTIKCQASQSINSWLSWYQQKPGQPPKLLIYQASKLASGVPSRFKGSGSGTEYTLTISDLKCADAATYYCQSYDYASNSGYVSNPFGGGTEVVVK

SEQ ID NO: 2 - protein, 117 aa, Oryctolagus cuniculus
  BINDING 26..33: note = Complementarity-determining region
  BINDING 51..58: note = Complementarity-determining region
  BINDING 96..106: note = Complementarity-determining region
QEHLVESEGGLFKPTDTLTLTCTVSGFSLSSYGVSWVRQAPGKGLEWIGVIGATGSAYYANWAKSRSTITRNTNLNTVTLKVTSLTAADTATYFCARDDVLTGMDLWGPGTLVTVSS

SEQ ID NO: 3 - DNA, 339 bp, Oryctolagus cuniculus
  source 1..339: mol_type = other DNA
gcacctgtgctgacccagacaccagcctccgtggaggcagctgtgggaggcacagtcaccatcaagtgccaggccagtcagagtattaatagttggttatcctggtatcagcagaaaccagggcagcctcccaagctcctgatctaccaggcatccaaactggcatctggggtcccatcgcggttcaaaggcagtggatctgggacagagtacactctcaccatcagcgacctgaagtgtgccgatgctgccacttattattgtcaaagctatgattatgctagtaatagtggatatgtgtctaatcctttcggcggagggaccgaggtggtggtcaaa

SEQ ID NO: 4 - DNA, 351 bp, Oryctolagus cuniculus
  source 1..351: mol_type = other DNA
caggagcatctggtggagtccgagggaggtctcttcaagccaacggataccctgacactcacctgcacagtctctggattctccctcagtagctatggagtgagctgggtccgccaggctccagggaaggggctggaatggatcggagtcattggtgctactggcagcgcatactacgcgaactgggcgaaaagccgatccaccatcaccagaaacaccaacctgaacacagtgactctgaaggtgaccagtctgacagccgcggacacggccacctatttctgtgcgagggatgatgtgcttactggtatggacctctggggcccagggaccctcgtcaccgtctcttca

SEQ ID NO: 5 - protein, 115 aa, Oryctolagus cuniculus
  BINDING 27..34: note = Complementarity-determining region
  BINDING 52..54: note = Complementarity-determining region
  BINDING 93..105: note = Complementarity-determining region
DGVMTQTPSSVSAAVGSTVTISCQASQSVFSNNYLSWYQQKPGQPPKLLIYTASSLASGVPSRFKGSGSGTQFTLTISDLECADAATYYCQCTYGKSGTDTYLNAFGGGTEVVVK

SEQ ID NO: 6 - protein, 119 aa, Oryctolagus cuniculus
  BINDING 25..33: note = Complementarity-determining region
  BINDING 48..59: note = Complementarity-determining region
  BINDING 97..108: note = Complementarity-determining region
QSVEESGGDLVKPEGSLTLTCTASGFSFSISYWICWVRQTPGKGLEWIGCIYGGSSGRPYYASWAKGRFTISKTSSTTVTLQMTSLTAADTAAYFCATTAYGGYGYGTWGPGTLVTVSS

SEQ ID NO: 7 - DNA, 345 bp, Oryctolagus cuniculus
  source 1..345: mol_type = other DNA
gatggcgtgatgacccagactccatcctccgtgtctgcagctgtgggaagcacagtcaccatcagttgccaggccagtcagagtgtttttagtaacaactacttatcctggtatcagcagaaaccagggcagcctcccaagctcctgatctatactgcatccagtctggcatctggggtcccatcgcggttcaaaggcagtggatctgggacacagttcactctcaccatcagcgacctggagtgtgccgatgctgccacttactactgtcaatgcacttatggaaagagtggtactgatacttatctaaatgctttcggcggagggaccgaggtggtggtcaaa

SEQ ID NO: 8 - DNA, 357 bp, Oryctolagus cuniculus
  source 1..357: mol_type = other DNA
cagtcggtggaggagtccgggggagacctggtcaagcctgagggatccctgacactcacctgcacagcctctggattctccttcagtatcagctattggatatgctgggtccgccagactccagggaaggggctggagtggatcggatgcatttatggtggtagtagtggtcgtccttactacgcgagctgggcgaaaggccgattcaccatctccaaaacctcgtcgaccacggtgactctgcaaatgactagtctgacagccgcggacacggccgcctatttctgtgcgaccactgcttatggtggttatggttatggtacgtggggcccaggcaccctggtcaccgtctcctca

SEQ ID NO: 9 - protein, 109 aa, Oryctolagus cuniculus
  BINDING 27..32: note = Complementarity-determining region
  BINDING 50..52: note = Complementarity-determining region
  BINDING 91..99: note = Complementarity-determining region
DVVMTQTPASVEAAVGGTVTIKCQASQSISNLLAWYQQKPGQPPKRLIYGASNLESGVPSRFKGSGSGTQFTLTISDLECADAATYYCQCSYDGSSPNAFGGGTEVVVE

SEQ ID NO: 10 - protein, 117 aa, Oryctolagus cuniculus
  BINDING 25..33: note = Complementarity-determining region
  BINDING 51..60: note = Complementarity-determining region
  BINDING 97..106: note = Complementarity-determining region
QSVEESGGDLVKPGASLTLTCTASGFSFSSRYYMCWVRQAPGKGLEWIGCIFGGSSGNTHYASWAKGRFTIAKTSSTTVTLRMTSLTAADTATYSCVRSDAWGVNLWGPGTLVTVSS

SEQ ID NO: 11 - DNA, 327 bp, Oryctolagus cuniculus
  source 1..327: mol_type = other DNA
gatgttgtgatgacccagactccagcctccgtggaggcagctgtgggaggcacagtcaccatcaagtgccaggccagtcagagcattagcaacctcttagcctggtatcaacagaaaccagggcagcctcccaagcgcctgatctatggtgcatccaatctggaatctggggtcccatcgcggttcaaaggcagtggatctgggacacagttcactctcaccatcagcgacctggagtgtgccgatgctgccacttactactgtcaatgtagttatgatggtagtagtcccaatgctttcggcggagggaccgaggtggtggtcgaa

SEQ ID NO: 12 - DNA, 351 bp, Oryctolagus cuniculus
  source 1..351: mol_type = other DNA
cagtcggtggaggagtccgggggagacctggtcaagcctggggcatccctgacactcacctgcacagcctctggattctccttcagtagccgctactatatgtgctgggtccgccaggctccagggaaggggctggagtggatcggctgtatttttggtggtagtagtggtaatactcactacgcgagctgggcgaaaggccgcttcaccatcgccaaaacctcgtcgaccacggtgactctgcgaatgaccagtctgacagccgcggacacggccacctattcttgtgtgagaagtgatgcctggggtgttaacttgtggggcccaggcaccctggtcaccgtctcctca

SEQ ID NO: 13 - protein, 113 aa, Oryctolagus cuniculus
  BINDING 27..32: note = Complementarity-determining region
  BINDING 50..52: note = Complementarity-determining region
  BINDING 89..103: note = Complementarity-determining region
APVLTHTAASVSEPVGGIVTINCQASQSIGSNLAWYQQKSGQPPKLLIYDASNLVSGVPSRFKGSRSGTEYTLTISDLECADAGTYYCQGYYYGSSTSYNLVSFGGGTEVVVK

SEQ ID NO: 14 - protein, 121 aa, Oryctolagus cuniculus
  BINDING 25..33: note = Complementarity-determining region
  BINDING 51..60: note = Complementarity-determining region
  BINDING 97..110: note = Complementarity-determining region
QSLEESGGGLVQPEGSLTLTCTASGFDLSRYYYIFWVRQAPGKGLEWIGCIYIGGNIITYYASWAKGRFTISKTSSPAVTLQMTSLTVADTATYFCARGTGYGGYGDVSLWGPGTLVTVSS

SEQ ID NO: 15 - DNA, 339 bp, Oryctolagus cuniculus
  source 1..339: mol_type = other DNA
gcacctgtgctgacccatactgcagcctccgtgtctgaacctgtgggaggcatagtcaccatcaactgccaggccagtcagagcattggtagtaatttagcctggtatcagcagaaatcagggcagcctcccaagctcctgatctatgatgcatccaatctggtatctggggtcccatcgcggttcaaaggcagcagatctgggacagagtacactctcaccatcagcgacctggagtgtgccgatgctggcacttactactgtcaaggctattattatggtagtagtactagctataatttagtgtctttcggcggagggaccgaggtggtggtcaaa

SEQ ID NO: 16 - DNA, 363 bp, Oryctolagus cuniculus
  source 1..363: mol_type = other DNA
cagtcgttggaggagtccgggggaggcctggtccagcctgagggatccctgacactcacctgcacagcttctggattcgacctcagtcgctactactacatattttgggtccgccaggctccagggaaggggctggagtggatcggatgcatttatattggtggtaatattatcacttactacgcgagctgggcgaaaggccgcttcaccatctccaaaacctcgtcgcccgcggtgactctgcaaatgaccagcctgacagtcgcggacacggccacctatttctgtgcgagagggactggttatggtggttatggtgatgttagcttgtggggcccaggcaccctggtcaccgtctcctca

SEQ ID NO: 17 - protein, 109 aa, Oryctolagus cuniculus
  BINDING 27..32: note = Complementarity-determining region
  BINDING 50..52: note = Complementarity-determining region
  BINDING 91..99: note = Complementarity-determining region
AAVMTQTPSPVSGVVGGTVTIKCQASQSIYGYFSWYQQKPGQPPKLLIYSASDLASGVPSRFKGSGSGTQFTLTISDLECADAATYYCQCSYDGSSPNAFGGGTEVVVK

SEQ ID NO: 18 - protein, 117 aa, Oryctolagus cuniculus
  BINDING 25..33: note = Complementarity-determining region
  BINDING 51..60: note = Complementarity-determining region
  BINDING 97..106: note = Complementarity-determining region
QSVEESGGDLVKPGASLTLTCTASGFSFSSRYYMCWVRQAPGKGLEWIACIFGGSSDNSYYARWAKGRFTISKTSSTTVTLRMTSLTAADTATYFCVRSDAWGVNLWGPGTLVTVSS

SEQ ID NO: 19 - DNA, 327 bp, Oryctolagus cuniculus
  source 1..327: mol_type = other DNA
gcagccgtgatgacccagactccatcccccgtgtctggagttgtgggaggcacagtcaccatcaagtgccaggccagtcagagcatttacggctacttttcttggtatcagcagaaaccagggcagcctcccaagctcctgatctattctgcatccgatctggcatctggggtcccatcgcggttcaaaggcagtggatctgggacacagttcactctcaccatcagcgacctggagtgtgccgatgctgccacttactactgtcaatgtagttatgatggtagtagtcccaatgctttcggcggagggaccgaggtggtggtcaaa

SEQ ID NO: 20 - DNA, 351 bp, Oryctolagus cuniculus
  source 1..351: mol_type = other DNA
cagtcggtggaggagtccgggggagacctggtcaagcctggggcatccctgacactcacctgcacggcctctggattctccttcagtagccgctattatatgtgctgggtccgccaggctccagggaaggggctggagtggatcgcatgtatttttggtggtagtagtgataatagttattacgcgaggtgggcgaaaggccgcttcaccatctccaaaacctcgtcgaccacggtgactctgcgaatgaccagtctgacggccgcggacacggccacttatttctgtgtgagaagtgatgcctggggtgttaacttgtggggcccaggcaccctggtcaccgtctcctca

SEQ ID NO: 21 - protein, 111 aa, Oryctolagus cuniculus
  BINDING 27..32: note = Complementarity-determining region
  BINDING 50..52: note = Complementarity-determining region
  BINDING 89..101: note = Complementarity-determining region
AAVLTQTPSSTSGPVGGTVTIKCQASQNIGSFLSWYQQKPGQPPKLLIYSASSLATGVPSRFSGSGSGTQFTLTISGVQCDDAATYYCLGFYAYRSDDGTAFGGGTEVVVK

SEQ ID NO: 22 - protein, 119 aa, Oryctolagus cuniculus
  BINDING 25..33: note = Complementarity-determining region
  BINDING 48..59: note = Complementarity-determining region
  BINDING 97..108: note = Complementarity-determining region
QSLEESGGDLVKPEGSLTLTCTASGFSFSISYWICWVRQTPGKGLEWIGCIYGGSSGRPYYASWAKGRFTISKTSSTTVTLQMTSLTAADTAAYFCATTAYGGYGYGTWGPGTLVTVSS

SEQ ID NO: 23 - DNA, 333 bp, Oryctolagus cuniculus
  source 1..333: mol_type = other DNA
gcagccgtgctgacccagactccatcctccacgtctggaccagtgggaggcacagtcaccatcaaatgccaggctagtcagaatattggtagtttcttatcctggtatcagcagaaaccagggcagcctcccaagctcctgatctattctgcatccagtctggcaactggggtcccatcgcggttcagcggcagtggatctgggacacagttcactctcaccattagcggcgtgcagtgtgacgatgctgccacttactactgtctaggtttttatgcttataggagtgatgacggtacggctttcggcggagggaccgaagtggtggtcaaa

SEQ ID NO: 24 - DNA, 357 bp, Oryctolagus cuniculus
  source 1..357: mol_type = other DNA
cagtcgttggaggagtccgggggagacctggtcaagcctgagggatccctgacactcacctgcacagcctctggattctccttcagtatcagctattggatatgctgggtccgccagactccagggaaggggctggagtggatcggatgcatttatggtggtagtagtggtcgtccttactacgcgagctgggcgaaaggccgattcaccatctccaaaacctcgtcgaccacggtgactctgcaaatgactagtctgacagccgcggacacggccgcctatttctgtgcgaccactgcttatggtggttatggttatggtacgtggggcccaggcaccctggtcaccgtctcctca

SEQ ID NO: 25 - protein, 113 aa, Oryctolagus cuniculus
  BINDING 27..32: note = Complementarity-determining region
  BINDING 50..52: note = Complementarity-determining region
  BINDING 91..103: note = Complementarity-determining region
AAVMTQTPSSLSAAVGGTVTIKCQASQSIRNQVSWYQQKPGQRPKLLIYRASTLESGVPSRFKGSGSGTDFTLTISDLECADAATYYCQCTYGSSSSNRYGNTFGGGTEVAVK

SEQ ID NO: 26 - protein, 119 aa, Oryctolagus cuniculus
  BINDING 25..33: note = Complementarity-determining region
  BINDING 51..59: note = Complementarity-determining region
  BINDING 96..108: note = Complementarity-determining region
QSLEESGGGLVQPEGSLTLTCTASGFSFSSSYYMCWVRQAPGKGLEWIGSIYTLSGSTAYASWAKGRFTISKTSSTTVTLQMTSLTAADTAMYFCARDSDGWGVSFNLWGPGTLVTVSS

SEQ ID NO: 27 - DNA, 339 bp, Oryctolagus cuniculus
  source 1..339: mol_type = other DNA
gcagccgtgatgacccagactccatcctccctgtctgccgctgtgggaggcacagtcaccatcaagtgccaggccagtcagagtattaggaatcaagtatcctggtatcagcagaaaccagggcagcgtcccaagctcctgatctacagggcatccactctggaatctggggtcccatcgcggttcaaaggcagtggatctgggacagacttcactctcaccatcagcgacctggagtgtgccgatgctgccacttactactgtcaatgcacttatggtagtagtagtagtaatcgttatggaaatactttcggcggagggaccgaggtggcggtcaaa

SEQ ID NO: 28 - DNA, 357 bp, Oryctolagus cuniculus
  source 1..357: mol_type = other DNA
cagtcgttggaggagtccgggggaggcctggtccagcctgagggatccctgacactcacctgcacagcttctggattctccttcagtagcagctactacatgtgctgggtccgccaggctccagggaaggggctggagtggatcggatccatttatactcttagtggtagcactgcctacgcgagctgggcgaaaggccgattcaccatctccaaaacctcgtcgaccacggtgactctgcaaatgaccagtctgacagccgcggacacggccatgtatttctgtgcgagagatagtgatggctggggtgtcagttttaacttgtggggcccaggcaccctggtcaccgtctcctca

SEQ ID NO: 29 - protein, 107 aa, Oryctolagus cuniculus
  BINDING 27..32: note = Complementarity-determining region
  BINDING 50..52: note = Complementarity-determining region
  BINDING 89..97: note = Complementarity-determining region
DVVMTQTPASVSAAVGATVTIKCQASETIGNNLAWYQQKPGQPPKVLMFLASTLASGVPSRFKGSRSGTEYTLTISGVQCDDAATYYCQSYLGTSYAFGGGTEVVVK

SEQ ID NO: 30 - protein, 120 aa, Oryctolagus cuniculus
  BINDING 25..33: note = Complementarity-determining region
  BINDING 48..59: note = Complementarity-determining region
  BINDING 96..109: note = Complementarity-determining region
QSLEESGGGLVQPEGTLTLTCTVSGFSFSSNYWICWVRQAPGKGLEWIACIVTNSGTTYYTSWAKGRLTLSKTSSTTVTLQMTSLTAADTATYFCARDNNSVTGVEFNLWGPGTLVTVSS

SEQ ID NO: 31 - DNA, 321 bp, Oryctolagus cuniculus
  source 1..321: mol_type = other DNA
gatgttgtgatgacccagactccagcctccgtgtctgcagctgtgggagccacagtcaccatcaagtgccaggccagtgagaccattggtaataatttagcctggtatcagcagaaaccagggcagcctcccaaggtcctgatgtttctggcatccacactggcatctggggtcccatcgcggttcaaaggcagtagatctgggacagagtacactctcaccatcagcggcgtgcagtgtgacgatgctgccacttactactgtcaaagctatctgggtactagttatgctttcggcggagggaccgaggtggtcgtcaaa

SEQ ID NO: 32 - DNA, 360 bp, Oryctolagus cuniculus
  source 1..360: mol_type = other DNA
cagtcgttggaggagtccgggggaggcctggtccagcctgagggaaccctgacactcacctgcacagtctctggattctccttcagtagcaactactggatatgctgggtccgccaggctccagggaaggggctggagtggatcgcatgcattgttactaatagtggtactacttactacacgagctgggcgaaaggccgactcaccctctccaaaacctcatcgaccacggtgactctacaaatgaccagtctgacagccgcggacacggccacctatttctgtgcgagagataataatagtgtgactggtgttgagtttaatttgtggggcccaggcaccctggtcaccgtctcctca

SEQ ID NO: 33 - protein, 112 aa, Oryctolagus cuniculus
  BINDING 27..34: note = Complementarity-determining region
  BINDING 52..54: note = Complementarity-determining region
  BINDING 91..102: note = Complementarity-determining region
DPMLTQTPSSKSVPVGGTVTINCQSSESVYDNNWLAWFQQKPGQPPKRLIYSASTLASGVSSRFKGSGSGTQFTLTISDVQCDDAATYYCTGYKSRTTDEIAFGGGTEVVVK

SEQ ID NO: 34 - protein, 119 aa, Oryctolagus cuniculus
  BINDING 26..33: note = Complementarity-determining region
  BINDING 51..58: note = Complementarity-determining region
  BINDING 96..108: note = Complementarity-determining region
QEQLEESEGGLFKPTDTLTLTCTVSGFSLSSYGVSWVRQAPGNGLEYIGFIGKTGSAYYASWAKSRSTITRNTTLNTVTLKMTSLTVADTATYFCARLGPGSIYYFDIWGPGTLVTVSS

SEQ ID NO: 35 - DNA, 336 bp, Oryctolagus cuniculus
  source 1..336: mol_type = other DNA
gaccctatgctgacccagactccatcttccaagtctgtccctgtgggaggcacagtcaccatcaattgccagtccagtgagagtgtttatgataacaactggttagcctggtttcagcagaagccagggcagcctcccaagcgcctgatctattctgcatccactctggcatctggggtctcatcgcgattcaaaggcagtggatctgggacacagttcactctcaccatcagcgacgtgcagtgtgacgatgctgccacttactactgtacaggatataaaagccgtactactgatgagattgctttcggcggagggaccgaggtggtggtcaaa

SEQ ID NO: 36 - DNA, 357 bp, Oryctolagus cuniculus
  source 1..357: mol_type = other DNA
caggagcagctggaggagtccgagggaggtctcttcaagccaacggataccctgacactcacctgcacagtctctggattctccctcagtagctatggagtgagctgggtccgccaggctccagggaacgggctggaatacatcggattcattggtaaaactggtagcgcatactacgcgagctgggcgaaaagccgatccaccatcaccagaaacaccaccctgaacacggtgactctgaaaatgaccagtctgacagtcgcggacacggccacctatttctgtgcgagattgggacctggtagtatttactattttgacatctggggcccaggcaccctggtcaccgtctcctca

SEQ ID NO: 37 - protein, 110 aa, Oryctolagus cuniculus
  BINDING 27..34: note = Complementarity-determining region
  BINDING 52..54: note = Complementarity-determining region
  BINDING 91..100: note = Complementarity-determining region
DGVMTQTPSSTSAAVGSTVTISCQSTQSIINNNWLAWYQQKPGHSPKLLIYDASTLASGVPSRFSGSGSGTQFTLTISDLECDDAATYYCAARYSGDIYTFGGGTEVVVR

SEQ ID NO: 38 - protein, 120 aa, Oryctolagus cuniculus
  BINDING 26..33: note = Complementarity-determining region
  BINDING 51..58: note = Complementarity-determining region
  BINDING 96..109: note = Complementarity-determining region
QEQLEESGGGLVKPTDTLTLTCTVSGFSLSTNSIFWVRQAPGNGLEWIGAISYAGGTAYASWAKSRSTITRNTNLNTVTLKMTSLTAADTATYFCGRGYVGYSRSKGDIWGPGTLVTVSS

SEQ ID NO: 39 - DNA, 330 bp, Oryctolagus cuniculus
  source 1..330: mol_type = other DNA
gatggcgtgatgacccagactccatcttccacgtctgcagctgtgggaagcacagtcaccatcagttgccagtccactcagagtattattaataataattggttagcctggtatcagcagaaaccagggcactctcctaagctcctgatctacgatgcatccactctggcatctggggtcccatcacggttcagcggcagtggatctgggacacagttcactctcaccatcagcgacctggagtgtgacgatgctgccacttactactgtgcagcccgttatagtggtgatatttatactttcggcggagggaccgaggtggtggtcaga

SEQ ID NO: 40 - DNA, 360 bp, Oryctolagus cuniculus
  source 1..360: mol_type = other DNA
caggagcagctggaggaatccgggggaggcctggtcaagccaacggataccctgacactcacctgcacagtctctggattctccctcagtaccaattcaattttctgggtccgccaggctccagggaacgggctggaatggatcggagccattagttatgctggtggcacagcctacgcgagctgggcgaaaagccgatccaccatcaccagaaacaccaacctgaacacggtgactctgaagatgactagtctgacagccgcggacacggccacctatttctgtgggagaggttatgttggttatagtcgtagtaagggggatatctggggcccaggcaccctggtcaccgtctcctca

SEQ ID NO: 41 - protein, 112 aa, Oryctolagus cuniculus
  BINDING 27..32: note = Complementarity-determining region
  BINDING 50..52: note = Complementarity-determining region
  BINDING 89..102: note = Complementarity-determining region
DVVMTQTPASVEAAVGGTVTIKCQATESIASWLAWYQQKAGQPPKLLIYEASKLESGVPSRFKGSGSGTEFTLTISDLECADAATYYCQSYLYSSSNTYGNSFGGGTEVVVK

SEQ ID NO: 42 - protein, 111 aa, Oryctolagus cuniculus
  BINDING 25..32: note = Complementarity-determining region
  BINDING 50..57: note = Complementarity-determining region
  BINDING 95..100: note = Complementarity-determining region
QSLEESEGGLFKPTDTLTVTCIVSGLSLSRNAMSWVRQAPGSGLEWIGVIVTYGDTYYASWAKSRSTITRNTNENTVTLKMTSLTAADTATYFCIGGLDIWGPGTLVTVSS

SEQ ID NO: 43 - DNA, 336 bp, Oryctolagus cuniculus
  source 1..336: mol_type = other DNA
gatgttgtgatgacccagactccagcctccgtggaggcagctgtgggaggcacagtcaccatcaagtgccaggccactgagagcattgccagttggttagcctggtatcagcagaaagcagggcagcctcccaagctcctgatctacgaagcatccaaactggaatctggggtcccatcgcggttcaaaggcagtggatctgggacagagttcactctcaccatcagcgacctggagtgtgccgatgctgccacttactactgtcaaagctatctttatagtagcagtaatacttatggtaattctttcggcggagggaccgaggtggtggtcaaa

SEQ ID NO: 44 - DNA, 333 bp, Oryctolagus cuniculus
  source 1..333: mol_type = other DNA
cagtcgttggaggagtccgagggaggtctcttcaagccaacggataccctgacagtcacctgtatagtctctggactctccctcagtaggaatgcaatgagctgggtccgccaggctccagggagcgggctggaatggatcggagtcattgtgacttatggtgacacatactacgcgagctgggcgaaaagccgatccaccatcaccagaaacaccaacgagaacacggtgactctgaaaatgaccagtctgacagccgcggacacggccacctatttttgtattggtggtcttgacatctggggcccaggcaccctggtcaccgtctcctca

SEQ ID NO: 45 - protein, 107 aa, Oryctolagus cuniculus
  BINDING 27..32: note = Complementarity-determining region
  BINDING 50..52: note = Complementarity-determining region
  BINDING 89..97: note = Complementarity-determining region
DVMMTQTPASVSEPVGGTVTIKCQASQNIGNNLAWYQQKPGQPPKLLIYLTSTLASGVPSRFKGSRSGTEFTLTISDLECADAATYFCQSYLYTTYAFGGGTEVVVK

SEQ ID NO: 46 - protein, 120 aa, Oryctolagus cuniculus
  BINDING 25..33: note = Complementarity-determining region
  BINDING 48..59: note = Complementarity-determining region
  BINDING 96..109: note = Complementarity-determining region
QSLEESGGGLVKPEGSLTLTCTASGFFFDRSYWICWVRQTPAKGLEWIACIVVSSGATYYASWAKGRLTLSKTSSTTVTLQMTSLTAADTAIYFCARDSSSTSGVEFNLWGPGTLVTVSS

SEQ ID NO: 47 - DNA, 321 bp, Oryctolagus cuniculus
  source 1..321: mol_type = other DNA
gatgtcatgatgacccagactccagcctccgtgtctgaacctgtgggaggcacagtcaccatcaagtgccaggccagtcagaacattggtaataatttagcctggtatcagcagaaaccagggcagcctcccaagctcctgatctatctgacatccactctggcatctggggtcccatcgcggttcaaaggcagtagatctgggacagagttcactctcaccatcagcgacctggagtgtgccgatgctgccacttacttctgtcaaagctatttatatactacttatgctttcggcggagggaccgaggtggtcgtcaaa

SEQ ID NO: 48 - DNA, 360 bp, Oryctolagus cuniculus
  source 1..360: mol_type = other DNA
cagtcgttggaggagtccgggggaggcctggtcaagcctgagggatccctgacactcacctgcacagcctctggtttcttcttcgatagaagctactggatatgctgggtccgccagactccagcaaaggggctggagtggatcgcatgcattgttgttagcagtggtgctacttactacgcgagctgggcgaaaggccgactcaccctctccaaaacctcgtcgaccacggtgactctacaaatgaccagtctgacagccgcggacacggccatctatttctgtgcgagagatagtagtagtactagtggtgttgagtttaatttgtggggcccaggcaccctggtcaccgtctcctca

SEQ ID NO: 49 - protein, 111 aa, Oryctolagus cuniculus
  BINDING 27..34: note = Complementarity-determining region
  BINDING 52..54: note = Complementarity-determining region
  BINDING 91..101: note = Complementarity-determining region
DGVMTQTPSSVSAAVGGTVTISCQSSQSVYKNNYLSWFQQKPGQPPKLLIYEISKLESGVPDRFSGSGSGTQFTLTISGVQCDDAATYYCLGGYDDDSDTSFGGGTEVVVK

SEQ ID NO: 50 - protein, 115 aa, Oryctolagus cuniculus
  BINDING 26..33: note = Complementarity-determining region
  BINDING 51..58: note = Complementarity-determining region
  BINDING 96..104: note = Complementarity-determining region
QEQLEESEGGLFKPTDTLTLTCTVSGFSLSVYALTWVRQAPGNGLEWIGAIGNSGNTYYASWAKSRSTITRNTNENTVTLKMTSLTAADTATYFCARGYVRINVWGPGTLVTVSS

SEQ ID NO: 51 - DNA, 333 bp, Oryctolagus cuniculus
  source 1..333: mol_type = other DNA
gatggcgtgatgacccagactccatcgtccgtgtctgcagctgtgggaggcacagtcaccatcagttgccagtccagtcagagtgtttataagaacaactacttatcctggtttcagcagaaaccagggcagcctcccaagctcctgatctacgaaatatccaaactggaatctggggtcccagataggttcagcggcagtggatctgggacacagttcactctcaccatcagcggcgtgcagtgtgacgatgctgccacttactactgtctaggcggttatgatgatgattctgatacttctttcggcggagggaccgaggtggtggtcaaa

SEQ ID NO: 52 - DNA, 345 bp, Oryctolagus cuniculus
  source 1..345: mol_type = other DNA
caggagcagctggaggagtccgagggaggtctcttcaagccaacggataccctgacactcacctgcacagtctctggattctccctcagtgtctatgctttaacctgggtccgccaggctccagggaacgggctggagtggatcggagccattggtaatagtggtaacacgtactacgcgagctgggcgaaaagccgatccaccatcaccagaaacaccaacgagaacacggtgactctgaaaatgaccagtctgacagccgcggacacggccacctatttctgtgcgagaggatatgtccgaattaacgtttggggcccaggcaccctggtcaccgtctcctcg

SEQ ID NO: 53 - protein, 107 aa, Oryctolagus cuniculus
  BINDING 27..32: note = Complementarity-determining region
  BINDING 54..56: note = Complementarity-determining region
  BINDING 89..97: note = Complementarity-determining region
DVVMTQTPASVSEPVGGTVTIKCQASQNIGNNLAWYQQKPGHPPKLLIYLASTLASGVPSRFKGSRSGTEFTLTISDLECADAATYYCQSYLYTTYAFGGGTEVVVK

SEQ ID NO: 54 - protein, 120 aa, Oryctolagus cuniculus
  BINDING 25..33: note = Complementarity-determining region
  BINDING 48..59: note = Complementarity-determining region
  BINDING 96..109: note = Complementarity-determining region
QSVEESGGDLVRPEGSLTLTCTASGFSLSSSYWICWVRQAPGKGLEWIACIVSNTGTTYYASWAKGRLTLSKTSSTTVTLQMTSLTAADTATYFCARDNNSKSGVEFNLWGPGTLVTVSS

SEQ ID NO: 55 - DNA, 321 bp, Oryctolagus cuniculus
  source 1..321: mol_type = other DNA
gatgttgtgatgacccagactccagcctccgtgtctgaacctgtgggaggcacagtcaccatcaagtgccaggccagtcagaacattggtaataatttagcctggtatcagcagaaaccagggcaccctcccaagctcctgatctatctggcatctactctggcatctggggtcccatcgcgattcaaaggcagtagatctgggacagagttcactctcaccatcagcgacctggagtgtgccgatgctgccacttactactgtcaaagctatttgtatactacttatgctttcggcggagggaccgaggtggtcgtcaaa

SEQ ID NO: 56 - DNA, 360 bp, Oryctolagus cuniculus
  source 1..360: mol_type = other DNA
cagtcggtggaggagtccgggggagacctggtcaggcctgagggatccctgacactcacctgcacagcttctggattctccctcagtagcagctactggatatgctgggtccgccaggctccaggaaaggggctagagtggatcgcatgcattgttagtaatactggtaccacttactacgcgagctgggcgaaaggccgactcaccctctccaaaacctcgtcgaccacggtgactctgcaaatgaccagtctgacagccgcggacacggccacctatttctgtgcgagagataataatagtaaaagtggtgttgagtttaatttgtggggcccaggcaccctggtcaccgtctcctca

SEQ ID NO: 57 - protein, 349 aa, synthetic construct
  SIGNAL 1..19
  REGION 344..349: note = PolyHis tag
MGWTLVFLFLLSVTAGVHSQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVGGSHRRLDKIEDERNLHEDFVFMKTIQRCNKGEGSLSLLNCKEIRSQFEGFVKDIMLNKEEPKKEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKLRGSHHHHHH

SEQ ID NO: 58 - protein, 342 aa, synthetic construct
  SIGNAL 1..19
MGWTLVFLFLLSVTAGVHSQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVGSGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 131 aa, synthetic construct
  SIGNAL 1..19
  REGION 126..131: note = PolyHis tail
MGWTLVFLFLLSVTAGVHSQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVRGSHHHHHH

SEQ ID NO: 60 - protein, 342 aa, synthetic construct
  SIGNAL 1..19
MGWTLVFLFLLSVTAGVHSQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVGGSPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - protein, 341 aa, synthetic construct
  SIGNAL 1..19
  REGION 336..341: note = PolyHis tail
MGWTLVFLFLLSVTAGVHSRAGGCNETGMLERLPLCGKAFADMMGKVDVWKWCNLSEFIVYYESFTNCTEMEANVVGCYWPNPLAQGFITGIHRQFFSNCTVDRVHLEDPPDEVGGSHRRLDKIEDERNLHEDFVFMKTIQRCNKGEGSLSLLNCKEIRSQFEGFVKDIMLNKEEPKKEINFEMQKGDQDPQIAAHLISEASSKSSSVLQWAKKGYYTMSNTLVTLENGKQLKVKRQGFYYIYAQVTFCSNQEPSSQAPFIASLCLKSSGGSERILLRAANARSSSKTCEQQSIHLGGVFELQADASVFVNVTDASQVNHGTGFTSFGLLKLRGSHHHHHH

SEQ ID NO: 62 - protein, 334 aa, synthetic construct
  SIGNAL 1..19
MGWTLVFLFLLSVTAGVHSRAGGCNETGMLERLPLCGKAFADMMGKVDVWKWCNLSEFIVYYESFTNCTEMEANVVGCYWPNPLAQGFITGIHRQFFSNCTVDRVHLEDPPDEVGSGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 123 aa, synthetic construct
  SIGNAL 1..19
  REGION 118..123: note = PolyHis Tail
MGWTLVFLFLLSVTAGVHSRAGGCNETGMLERLPLCGKAFADMMGKVDVWKWCNLSEFIVYYESFTNCTEMEANVVGCYWPNPLAQGFITGIHRQFFSNCTVDRVHLEDPPDEVRGSHHHHHH

SEQ ID NO: 64 - protein, 334 aa, synthetic construct
  SIGNAL 1..19
MGWTLVFLFLLSVTAGVHSRAGGCNETGMLERLPLCGKAFADMMGKVDVWKWCNLSEFIVYYESFTNCTEMEANVVGCYWPNPLAQGFITGIHRQFFSNCTVDRVHLEDPPDEVGGSPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 65 - protein, 122 aa, synthetic construct
  BINDING 26..34: note = Complementarity-determining region - IGMT
  BINDING 32..36: note = Complementarity-determining region - Kabat
  BINDING 51..67: note = Complementarity-determining region - Kabat
  BINDING 52..59: note = Complementarity-determining region - IMGT
  BINDING 98..111: note = Complementarity-determining region - IMGT
  BINDING 100..111: note = Complementarity-determining region - Kabat
EVQLVESGGGLVQPGGSLRLSCAASGFSLSSSYWICWVRQAPGKGLEWIACIVSNTGTTYYASWAKGRFTLSKDTSKTTLTLQMNSLRAEDTAVYFCARDNNSKSGVEFNLWGQGTLVTVSS

SEQ ID NO: 66 - protein, 122 aa, synthetic construct
  BINDING 26..34: note = Complementarity-determining region - IMGT
  BINDING 32..36: note = Complementarity-determining region - Kabat
  BINDING 51..67: note = Complementarity-determining region - Kabat
  BINDING 52..59: note = Complementarity-determining region - IMGT
  BINDING 98..110: note = Complementarity-determining region - IMGT
  BINDING 100..111: note = Complementarity-determining region - Kabat
ELQLVESGGAVVQPGESLRLSCAASGFSLSSSYWICWVRQAPGRGLEWVACIVSNTGTTYYASWAKGRLTISKDTSSNTLYLQMNSLRAEDTAMYFCARDNNSKSGVEFNLWGQGTLVTVSS

SEQ ID NO: 67 - protein, 121 aa, synthetic construct
  BINDING 26..34: note = Complementarity-determining region - IMGT
  BINDING 32..36: note = Complementarity-determining region - Kabat
  BINDING 51..67: note = Complementarity-determining region - Kabat
  BINDING 52..59: note = Complementarity-determining region - IMGT
  BINDING 97..109: note = Complementarity-determining region - IMGT
  BINDING 99..110: note = Complementarity-determining region - Kabat
ELQLVESGGAVVQPGESLRLSCAASGFSLSSSYWICWVRQAPGRGLEWVGCIVSNTGTTYYASWAKGRLTISKTSSNTLYLQMNSLRAEDTAMYFCARDNNSKSGVEFNLWGQGTLVTVSS

SEQ ID NO: 68 - protein, 122 aa, synthetic construct
  BINDING 26..34: note = Complementarity-determining region - IMGT
  BINDING 32..36: note = Complementarity-determining region - Kabat
  BINDING 51..67: note = Complementarity-determining region - Kabat
  BINDING 52..59: note = Complementarity-determining region - IMGT
  BINDING 98..111: note = Complementarity-determining region - IMGT
  BINDING 100..111: note = Complementarity-determining region - Kabat
EVQLVESGGDLAQPGGSLRLSCTVSGFSLSSSYWICWVRQAPGKGLEWVACIVSNTGTTYYASWAKGRLTISRDNSKNTVYLQMTSLRAEDTALYFCARDNNSKSGVEFNLWGQGTLVTVSS

SEQ ID NO: 69 - protein, 122 aa, synthetic construct
  BINDING 26..34: note = Complementarity-determining region - IMGT
  BINDING 32..36: note = Complementarity-determining region - Kabat
  BINDING 51..67: note = Complementarity-determining region - Kabat
  BINDING 52..59: note = Complementarity-determining region - IMGT
  BINDING 98..111: note = Complementarity-determining region - IMGT
  BINDING 100..111: note = Complementarity-determining region - Kabat
QVQLVQSGGEVKQPGASVKVSCTASGFSLSSSYWICWVRQAPGKGLEWMGCIVSNTGTTYYASWAKGRFVLSKDTSASTATLQISSLKAADTATYFCARDNNSKSGVEFNLWGPGTLVTVSS

SEQ ID NO: 70 - protein, 107 aa, synthetic construct
  BINDING 24..34: note = Complementarity-determining region - Kabat
  BINDING 27..32: note = Complementarity-determining region - IMGT
  BINDING 50..52: note = Complementarity-determining region - IMGT
  BINDING 50..56: note = Complementarity-determining region - Kabat
  BINDING 89..97: note = Complementarity-determining region - IMGT
  BINDING 89..97: note = Complementarity-determining region - Kabat
DIQMTQSPSTLSASVGDRVTITCQASQNIGNNLAWYQQKPGHAPKLLIYLASTLASGVPSRFSGSGSGTEFTLTISSLQPDDFATYYCQSYLYTTYAFGQGTKLEIK

SEQ ID NO: 71 - protein, 107 aa, synthetic construct
  BINDING 24..34: note = Complementarity-determining region - Kabat
  BINDING 27..32: note = Complementarity-determining region - IMGT
  BINDING 50..52: note = Complementarity-determining region - IMGT
  BINDING 50..56: note = Complementarity-determining region - Kabat
  BINDING 89..97: note = Complementarity-determining region - IMGT
  BINDING 89..97: note = Complementarity-determining region - Kabat
DIQMTQSPSSLSASVGDRVTIKCQASQNIGNNLAWYQQKPGKVPKLLIYLASTLASGVPSRFKGSRSGTDFTLTISSLQPEDVATYYCQSYLYTTYAFGGGTKVEIK

SEQ ID NO: 72 - protein, 107 aa, synthetic construct
  BINDING 24..34: note = Complementarity-determining region - Kabat
  BINDING 50..56: note = Complementarity-determining region - Kabat
  BINDING 27..32: note = Complementarity-determining region - IMGT
  BINDING 50..52: note = Complementarity-determining region - IMGT
  BINDING 89..97: note = Complementarity-determining region - IMGT
  BINDING 89..97: note = Complementarity-determining region - Kabat
DVVMTQSPSTVSASVGDRVTLKCQASQNIGNNLAWYQQKPGHPPKLLIYLASTLASGVPDRFSGSGSGTDFTLTISSLEAADAATYYCQSYLYTTYAFGQGTKVEIK

SEQ ID NO: 73 - protein, 107 aa, synthetic construct
  BINDING 24..34: note = Complementarity-determining region - Kabat
  BINDING 27..32: note = Complementarity-determining region - IGMT
  BINDING 50..52: note = Complementarity-determining region - IMGT
  BINDING 50..56: note = Complementarity-determining region - Kabat
  BINDING 89..97: note = Complementarity-determining region - IMGT
  BINDING 89..97: note = Complementarity-determining region - Kabat
EIVMTQSPATLSVSPGERATLKCQASQNIGNNLAWYQQKPGHPPRLLIYLASTLASGIPARFKGSRSGTEFTLTISDLESADAATYYCQSYLYTTYAFGGGTEVVVK

SEQ ID NO: 74 - protein, 107 aa, synthetic construct
  BINDING 24..34: note = Complementarity-determining region - Kabat
  BINDING 50..56: note = Complementarity-determining region - Kabat
  BINDING 27..32: note = Complementarity-determining region - IMGT
  BINDING 50..52: note = Complementarity-determining region - IGMT
  BINDING 89..97: note = Complementarity-determining region - IMGT
  BINDING 89..97: note = Complementarity-determining region - Kabat
DIQMTQSPSSLSASVGDRVTIKCQASQNIGNNLAWYQQKPGHPPKLLIYLASTLASGVPSRFKGSRSGTDFTLTISDLEPADAATYYCQSYLYTTYAFGGGTEVVVK

SEQ ID NO: 75 - protein, 119 aa, synthetic construct
  BINDING 25..33: note = Complementarity-determining region
  BINDING 48..60: note = Complementarity-determining region
  BINDING 97..108: note = Complementarity-determining region
QSLEESGGDLVKPEGSLTLTCTASGFSFSSSYYXCWVRQAPGKGLEWIGCIYGGSSGXTYYASWAKGRFTISKTSSTTVTLQMTSLTAADTATYFCARDAXGGXGVNLWGPGTLVTVSS

SEQ ID NO: 76 - protein, 111 aa, synthetic construct
  BINDING 27..32: note = Complementarity-determining region
  BINDING 50..52: note = Complementarity-determining region
  BINDING 89..101: note = Complementarity-determining region
AAVMTQTPSSVSAAVGGTVTIKCQASQSIXXXLSWYQQKPGQPPKLLIYXASXLASGVPSRFKGSGSGTQFTLTISDLECADAATYYCQCXYXGSSXXXNAFGGGTEVVVK

SEQ ID NO: 77 - protein, 119 aa, synthetic construct
  BINDING 25..32: note = Complementarity-determining region
  BINDING 47..57: note = Complementarity-determining region
  BINDING 95..108: note = Complementarity-determining region
QSLEESGGGLVKPTDTLTLTCTVSGFSLSSYWICWVRQAPGXGLEWIGCIXNSGXTYYASWAKSRSTITRNTXSNTVTLKMTSLTAADTATYFCARGXXSXXXXXFNXWGPGTLVTVSS

SEQ ID NO: 78 - protein, 107 aa, synthetic construct
  BINDING 27..32: note = Complimentary Determining Region
  BINDING 50..52: note = Complimentary Determining Region
  BINDING 89..97: note = Complimentary Determining Region
DVVMTQTPASVSAAVGGTVTIKCQASQSIXNXLAWYQQKPGQPPKLLIYLASTLASGVPSRFKGSGSGTEFTLTISDLECDDAATYYCQSYLYTXYAFGGGTEVVVK